(19) United States
(12) Patent Application Publication
(10) Pub. No.: US 2023/0122991 A1
(43) Pub. Date: Apr. 20, 2023

(54) GROWTH FACTOR FORMULATION FOR CONDITION ASSOCIATED WITH OTIC EVENT

(71) Applicant: OTONOMY, INC., San Diego, CA (US)

(72) Inventors: Alan FOSTER, San Diego, CA (US); Bonnie Elizabeth JACQUES, San Diego, CA (US); Fabrice PIU, San Diego, CA (US)

(21) Appl. No.: 17/794,935

(22) PCT Filed: Jan. 22, 2021
(86) PCT No.: PCT/US2021/014781

§ 371 Date: Jul. 22, 2022

Related U.S. Application Data
(60) Provisional application No. 62/965,758, filed on Jan. 24, 2020.

(51) Int. Cl. A61K 38/18; A61K 9/00; A61P 27/16; A61K 9/06; A61K 47/34; A61K 47/14

(52) U.S. Cl. CPC A61K 38/185; A61K 9/0046; A61P 27/16; A61K 9/06; A61K 47/34; A61K 47/14

(57) ABSTRACT

Disclosed herein are otic formulations and compositions comprising growth factors. These otic formulations and compositions allow for the delivery of the growth factor to the outer, middle, and/or inner ear for the treatment of otic diseases and disorders.

### BACKGROUND OF THE DISCLOSURE

Vertebrates have a pair of ears, placed symmetrically on opposite sides of the head. The ear serves as both the sense organ that detects sound and the organ that maintains balance and body position. The ear is generally divided into three portions: the outer ear, auris media (or middle ear), and the auris interna (or inner ear).

### SUMMARY OF THE DISCLOSURE

Described herein, in some embodiments, are otic formulations comprising growth factors for drug delivery into the outer, middle, and/or inner ear, including the cochlea and vestibular labyrinth, including growth factor formulations that are useful for treating or ameliorating conditions associated with otic events.

One aspect of the present disclosure is directed to a method of treating an otic disease or condition associated with a synapses-affecting event in a subject in need thereof, the method comprising administering an otic formulation comprising a therapeutically effective amount of a growth factor and an auris-acceptable vehicle within 14 days after onset of the synapses-damaging event, wherein the otic formulation is formulated to provide sustained release of the growth factor into the inner ear to promote formation of synapses or repaire damaged synapses.

In some embodiments, the growth factor is brain-derived neurotrophic factor (BDNF), ciliary neurotrophic factor (CNTF), glial cell-line derived neurotrophic factor (GDNF), neurotrophin-3, neurotrophin-4, or any combination thereof.

In some embodiments, the growth factor is brain-derived neurotrophic factor (BDNF).

In some embodiments, the auris-acceptable gel comprises a copolymer of polyoxyethylene and polyoxypropylene.

In some embodiments, the copolymer of polyoxyethylene and polyoxypropylene is poloxamer 407.

In some embodiments, the otic formulation comprises from about 15 wt % to about 17 wt % poloxamer 407.

In some embodiments, the otic formulation has an osmolarity from about 100 mOsm/L to about 1000 mOsm/L.

In some embodiments, the otic formulation has a pH from about 7.0 to about 8.0.

In some embodiments, the otic formulation comprises about 0.0001% to about 1% by weight of the growth factor.

In some embodiments, the otic formulation comprises between about 0.005% to about 0.5% by weight of the growth factor.

In some embodiments, the otic formulation provides sustained release of the growth factor over a period of at least 3 days.

In some embodiments, the growth factor is dissolved in the otic formulation.

In some embodiments, the otic formulation is administered through intratympanic injection.

In some embodiments, the otic formulation is deposited on or close to the round window membrane of the subject.

In some embodiments, the otic disease or condition is hearing loss or hearing impairment.

In some embodiments, the synapses-affecting event is selected from the group consisting of: head trauma, traumatic brain injury (TBI), acoustic trauma, cochlear implant surgery, sudden sensorineural hearing loss, drug-induced ototoxicity, and excitotoxicity.

In some embodiments, the drug-induced ototoxicity is chemotherapy-induced ototoxicity.

In some embodiments, the drug-induced ototoxicity is cisplatin-induced ototoxicity.

In some embodiments, the drug-induced ototoxicity is antibiotic-induced ototoxicity.

In some embodiments, the otic formulation repairs ribbon synapses.

In some embodiments, the otic formulation is administered within 7 days after onset of the synapses-damaging event.

In some embodiments, the otic formulation is administered within 3 days after onset of the synapses-damaging event.

In some embodiments, the otic formulation is administered within 1 day after onset of the synapses-damaging event.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates the anatomy of the ear.

FIG. 2 shows the visualization of synapses on hair cells.

FIG. 3A shows that treatment with sustained release BDNF formulations leads to improved hearing function (wave I amplitude) in noise exposed adult rats compared to vehicle treatment. FIG. 3B shows that treatment with sustained release BDNF formulations leads to an increased number of ribbon synapses per inner hair cell in noise exposed adult rats compared to vehicle treatment.

FIG. 4 shows the perilymph concentrations of BDNF following a single IT injection in rats in poloxamer 407 formulation. BDNF was administered at the indicated doses. Perilymph BDNF is in arbitrary units.

FIGS. 5A, 5B, and 5C show perilymph concentrations of BDNF following a single IT injection in rats in MCT formulations. BDNF was administered at a dose of 0.15% (1.5 mg/ml) in the MCT formulation containing different polymers: PVP (polyvinylpyrrolidone) at 2 or 10% (FIG. 5A); 0.15% carbomer (FIG. 5B); or P407 (poloxamer 407) at 10% (FIG. 5C). Perilymph BDNF is in arbitrary units.

FIG. 6A schematically illustrates details of Example C. FIG. 6B shows DAPI-stained neurofilaments. FIG. 6C shows spiral ganglion neuron (SGN) survival normalized to 1 nM NT-3 for treatment with various neurotrophins and antibodies. FIG. 6D shows SGN survival normalized to 1 nM NT-3 for treatment at various concentrations of neurotrophins and antibodies.

FIGS. 7A-7D show changes in the complexities of SGNs upon treatment with various agents. FIG. 7A shows SGNs immunostained for neurofilament and DAPI and imaged at 20×. FIG. 7B shows roots (indicated with asterices), extremities (indicated with white dots), nodes (indicated with arrows), segments (indicated with discrete sections of neurite between nodes or extremities), and total neurite length measured for each neuron.

FIG. 7C shows the percentage of SGNs having various numbers of roots upon treatment with 10 nM BDNF, 1 nM NT-3, or 1 nM M3 compared to the IgG-treated control (*p<0.05, **p<0.005, and p<0.001). FIG. 7D shows total neurite length (left panel) as well as total number of extremities, segments, and nodes per cell upon treatment with BDNF, NT-3, or M3.

FIG. 8A schematically depicts the experimental scheme of Example E. FIG. 8B shows stained neurofilaments for samples treated with no neurotrophin/antibody (upper left image), 10 nM BDNF (upper right image), 10 nM NT-3 (middle left image), 100 nM NT-3 (middle right image), 10 nM M3 (lower left image), and 100 nM M3 (lower right image). FIGS. 8C and 8D show neurites per tissue at various concentrations of NT-3 (FIG. 8C) and BDNF (FIG. 8D). FIG. 8E shows neurites per tissue at various concentrations of hIgG4 (left) and M3 (right). FIG. 8F shows image analysis of explants. FIG. 8G shows the effect of BDNF, NT-3, and M3 on neurite complexity of SGN explants compared to IgG-treated or untreated controls.

FIG. 9A schematically depicts the experimental scheme of Example F. FIG. 9B shows estimation of type I SGNs based on counting inner hair cells (IHCs; triangles) and subtracting the number of fibers passing through to the outer hair cells (OHCs; circles). FIG. 9C shows SGN fibers/IHC normalized to CTL for various agents for no excitotoxin and 72 hour culture (left panel) and excitotoxin treatment and 18 hour culture (right panel). FIG. 9D shows images of cochlear explants taken under various conditions. FIG. 9E shows an image of cochlear explants with PSD-95, CtBP2, neurofilament (lines), and Myo7a. FIG. 9F shows puncta (PSD95)/IHC normalized to CTL for various Trk agonists for no excitotoxin and 72 hour culture (left panel) and excitotoxin and 72 hour culture (right panel) (*p<0.05 (CTL vs. NK)).

FIG. 10 schematically illustrates non-invasive intratympanic delivery of an agent such as BDNF to the inner ear.

FIG. 11 shows BDNF in perilymp (pg/ml) at various times post intratympanic injection for various concentrations of BDNF in P407.

FIG. 12A shows ABR threshold shift (dB SPL) at various frequencies for P407 vehicle alone and 0.05 mg/ml BDNF in P407. FIG. 12B shows ABR wave components (left panel) and noise-induced wave I deficits (right panel). FIG. 12C shows ABR wave I amplitudes measured in adult male rats after BDNF or vehicle treatment following noise exposure or in naive undamaged ears.

FIG. 13A shows inner hair cell innervation (upper panel) and hair cell ribbon synapses (lower panel). FIG. 13B shows high-magnification views of inner hair cells from the 40 kHz region from adult rats after BDNF or vehicle treatment following noise damage, or in a naive undamaged cochlea. Digital X-compressed cross-sections are shown at right.

FIG. 13C shows increased synaptic punca (defined by co-expression of CtBp2 and GluR2) per inner hair cell for each frequency region throughout the cochlea following BDNF treatment. FIG. 13D shows the cochlear frequency mapping.

FIG. 14 schematically depicts the study design of Example H.

FIG. 15 shows perilymph concentrations of BDNF following a single intratympanic injection in cats of BDNF poloxamer 407 formulation.

FIG. 16-19 shows ABR, wave I amplication, and synaptic punctae of rats receiving intratympanic injection of various concentrations of BDNF in P407 following a single intratympanic injection.

FIG. 20-22 shows ABR, wave I amplication, and synaptic punctae of rats receiving intratympanic injection of 0.005% BDNF in P407 after 1, 3, 7, or 14 days after noise exposure.

### DETAILED DESCRIPTION

Systemic administration of active agents is, in some instances, ineffectual in the treatment of diseases that affect inner ear structures. The cochlear canals and the cochlea, for example, are isolated from the circulatory system limiting systemic delivery of active agents to target sites in the inner ear. In some instances, systemic drug administration creates a potential inequality in drug concentration with higher circulating levels in the serum, and lower levels in the target auris interna organ structures. In certain instances, large amounts of drug are required to overcome this inequality in order to deliver sufficient, therapeutically effective quantities of a drug to auditory structures. In some instances, systemic drug administration also increases the likelihood of secondary systemic accumulation and consequent adverse side effects.

Currently available treatment for inner ear diseases also carries the risk of attendant side effects. For example, available methods require multiple daily doses (e.g., intratympanic injection or infusion) of drugs. In certain instances, multiple daily intratympanic injections cause patient discomfort and non-compliance. In certain instances, delivery of active agents to the inner ear via otic drops administered in the ear canal or via intratympanic injection is hindered by the biological barrier presented by the tympanic membrane the oval window membrane and/or the round window membrane. In some instances, delivery of active agents to the inner ear via otic drops or intratympanic injection causes osmotic imbalance in inner ear structures, introduces infections or other immune disorders as a result of microbial or endotoxin presence, or results in permanent structural damage (e.g. perforation of the tympanic membrane), resulting in hearing loss and the like.

Intratympanic injection of therapeutic agents is the technique of injecting a therapeutic agent behind the tympanic membrane into the auris media and/or auris interna. Some challenges remain with intratympanic injections. For example, access to the round window membrane, the site of drug absorption into the auris interna, is challenging in some instances. In addition, current regimens using intratympanic injections do not address changing the osmolarity and pH of the perilymph and endolymph, and introducing pathogens and endotoxins that directly or indirectly damage inner ear.

Provided herein in one aspect are otic formulations and compositions comprising a therapeutically effective amount of an active agent, such as a growth factor. In some embodiments, the growth factor is brain-derived neurotrophic factor (BDNF), ciliary neurotrophic factor (CNTF), glial cell-line derived neurotrophic factor (GDNF), neurotrophin-3, neurotrophin-4, or any combination thereof. In some embodiments, the growth factor is brain-derived neurotrophic factor (BDNF). In some embodiments, the otic formulations are auris-acceptable gels. In some embodiments, the otic formulations are triglyceride based auris-acceptable formulations.

These otic pharmaceutical formulations are suitable for drug delivery into the external, middle and/or inner ear. In some instances, these otic pharmaceutical formulations and compositions are suitable for administration to humans. In some instances, the otic formulations and compositions disclosed herein also meet stringent criteria for pH, osmolarity, ionic balance, sterility, endotoxin, and/or pyrogen levels. In some instances, the otic formulations and compositions are compatible with the microenvironment of the inner ear (e.g., the perilymph).

Accordingly, provided herein, in certain embodiments, are otic formulations and compositions that are controlled release auris-acceptable formulations and compositions that locally treat auris target structures and provide extended exposure of otic active agents to the target auris structures. In certain embodiments, the otic formulations and compositions described herein are designed for stringent osmolarity and pH ranges that are compatible with auditory structures and/or the endolymph and perilymph. In some embodiments, the otic formulations and compositions described herein are controlled release formulations that provide extended release for a period of at least 3 days and meet stringent sterility requirements. In some instances, otic formulations and compositions described herein contain lower endotoxin levels (e.g. <0.5 EU/mL when compared to typically acceptable endotoxin levels of 0.5 EU/mL. In some instances, the otic formulations and compositions described herein contain low levels of colony forming units (e.g., <50 CFUs) per gram of the formulation or composition. In some instances, the otic formulations or compositions described herein are substantially free of pyrogens and/or microbes. In some instances the otic formulations or compositions described herein are formulated to preserve the ionic balance of the endolymph and/or the perilymph.

In some instances, local administration of the otic formulations and compositions described herein avoids potential adverse side effects as a result of systemic administration of active agents. In some instances, the locally applied otic formulations and compositions described herein are compatible with auris structures. Such compatible auris structures include those associated with the outer, middle, and/or inner ear. In some embodiments, the otic formulations and compositions are administered either directly to the desired auris structure, e.g. the cochlear region, or administered to a structure in direct communication with areas of the auris structure; in the case of the cochlear region, for example, including but not limited to the round window membrane, the crista fenestrae cochleae or the oval window membrane.

In certain instances, the otic formulations and compositions disclosed herein controlled release formulations or compositions that provide a constant rate of release of a drug from the formulation and provide a constant prolonged source of exposure of an otic active agent to the inner ear of an individual or patient suffering from an otic disorder, reducing or eliminating any variabilities associated with other methods of treatment (such as, e.g., otic drops and/or multiple intratympanic injections).

In some embodiments, the otic formulations and compositions described herein provide extended release of the active ingredient(s) into the external ear. In some embodiments, the otic formulations and compositions described herein provide extended release of the active ingredient(s) into the middle and/or inner ear (auris interna), including the cochlea and vestibular labyrinth. In some embodiments, the otic formulations and compositions further comprise an immediate or rapid release component in combination with a controlled release component.

### Certain Definitions

The term “auris-acceptable” with respect to a formulation, composition or ingredient, as used herein, includes having no persistent detrimental effect on the auris externa (or external ear or outer ear), auris media (or middle ear) and/or the auris interna (or inner ear) of the subject being treated. By “auris-pharmaceutically acceptable,” as used herein, refers to a material, such as a carrier or diluent, which does not abrogate the biological activity or properties of the compound in reference to the auris externa (or external ear or outer ear), auris media (or middle ear) and/or the auris interna (or inner ear), and is relatively or is reduced in toxicity to the auris externa (or external ear or outer ear), auris media (or middle ear) and the auris interna (or inner ear), i.e., the material is administered to an individual without causing undesirable biological effects or interacting in a deleterious manner with any of the components of the composition in which it is contained.

As used herein, amelioration or lessening of the symptoms of a particular otic disease, disorder or condition by administration of a particular compound or pharmaceutical composition refers to any decrease of severity, delay in onset, slowing of progression, or shortening of duration, whether permanent or temporary, lasting or transient that is attributed to or associated with administration of the compound or composition.

As used herein, the term “antimicrobial agent” refers to compounds that inhibit the growth, proliferation, or multiplication of microbes, or that kill microbes. Suitable “antimicrobial agents” are antibacterial agents (effective against bacteria), antiviral agents (effective against viruses), antifungal agents (effective against fungi), antiprotozoal (effective against protozoa), and/or antiparasitic to any class of microbial parasites. “Antimicrobial agents” work by any suitable mechanism against the microbes, including by being toxic or cytostatic.

“Antioxidants” are auris-pharmaceutically acceptable antioxidants, and include, for example, butylated hydroxytoluene (BHT), sodium ascorbate, ascorbic acid, sodium metabisulfite and tocopherol. In certain embodiments, antioxidants enhance chemical stability where required. Antioxidants are also used to counteract the ototoxic effects of certain therapeutic agents.

The term “auris-acceptable penetration enhancer” with respect to a formulation, composition or ingredient, as used herein, refers to the property of reducing barrier resistance.

“Auris externa” refers to the external (or outer) ear, and includes the pinna and the external auditory canal (EAC).

“Auris interna” refers to the inner ear, including the cochlea and the vestibular labyrinth, and the round window that connects the cochlea with the middle ear.

“Auris-interna bioavailability” or “Auris media bioavailability” refers to the percentage of the administered dose of compounds disclosed herein that becomes available in the inner or middle ear, respectively, of the animal or human being studied.

“Auris media” refers to the middle ear, including the tympanic cavity, auditory ossicles and oval window, which connects the middle ear with the inner ear.

“Auris-interna bioavailability” refers to the percentage of the administered dose of compounds disclosed herein that becomes available in the inner ear of the animal or human being studied.

“Balance disorder” refers to a disorder, illness, or condition which causes a subject to feel unsteady, or to have a sensation of movement. Included in this definition are dizziness, vertigo, disequilibrium, and pre-syncope. Diseases which are classified as balance disorders include, but are not limited to, Ramsay Hunt's Syndrome, Méniére's Disease, mal de debarquement, benign paroxysmal positional vertigo, labyrinthitis, and presbycusis.

“Blood plasma concentration” refers to the concentration of compounds provided herein in the plasma component of blood of a subject.

“Carrier materials” are excipients that are compatible with the otic agent, the auris media, the auris interna and the release profile properties of the auris-acceptable pharmaceutical formulations. Such carrier materials include, e.g., binders, suspending agents, disintegration agents, filling agents, surfactants, solubilizers, stabilizers, lubricants, wetting agents, diluents, and the like. “Auris-pharmaceutically compatible carrier materials” include, but are not limited to, acacia, gelatin, colloidal silicon dioxide, calcium glycerophosphate, calcium lactate, maltodextrin, glycerine, magnesium silicate, polyvinylpyrrolidone (PVP), cholesterol, cholesterol esters, sodium caseinate, soy lecithin, taurocholic acid, phosphatidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, alginate, carbomer, hyaluronic acid (HA), poloxamer, dextran, and the like.

“Drug absorption” or “absorption” refers to the process of movement of the otic agent from the localized site of administration, by way of example only, the round window membrane of the inner ear, and across a barrier (the round window membranes, as described below) into the auris interna or inner ear structures. The terms “co-administration” or the like, as used herein, are meant to encompass administration of the otic agent to a single patient, and are intended to include treatment regimens in which the otic agents are administered by the same or different route of administration or at the same or different time.

The terms “effective amount” or “therapeutically effective amount,” as used herein, refer to a sufficient amount of the otic agent being administered that would be expected to relieve to some extent one or more of the symptoms of the disease or condition being treated. For example, the result of administration of the otic agents disclosed herein is reduction and/or alleviation of the signs, symptoms, or causes of any one of the diseases or conditions disclosed herein. For example, an “effective amount” for therapeutic uses is the amount of the otic agent, including a formulation as disclosed herein required to provide a decrease or amelioration in disease symptoms without undue adverse side effects. The term “therapeutically effective amount” includes, for example, a prophylactically effective amount. An “effective amount” of a otic agent composition disclosed herein is an amount effective to achieve a desired pharmacologic effect or therapeutic improvement without undue adverse side effects. It is understood that “an effective amount” or “a therapeutically effective amount” varies, in some embodiments, from subject to subject, due to variation in metabolism of the compound administered, age, weight, general condition of the subject, the condition being treated, the severity of the condition being treated, and the judgment of the prescribing physician. In some instances, it is also understood that “an effective amount” in an extended-release dosing format differs from “an effective amount” in an immediate-release dosing format based upon pharmacokinetic and pharmacodynamic considerations.

The terms “enhance” or “enhancing” refers to an increase or prolongation of either the potency or duration of a desired effect of the otic agent, or a diminution of any adverse symptomatology. For example, in reference to enhancing the effect of the otic agents disclosed herein, the term “enhancing” refers to the ability to increase or prolong, either in potency or duration, the effect of other therapeutic agents that are used in combination with the otic agents disclosed herein. An “enhancing-effective amount,” as used herein, refers to an amount of an otic agent or other therapeutic agent that is adequate to enhance the effect of another therapeutic agent or otic agent in a desired system. When used in a patient, amounts effective for this use will depend on the severity and course of the disease, disorder or condition, previous therapy, the patient's health status and response to the drugs, and the judgment of the treating physician.

The term “inhibiting” includes preventing, slowing, or reversing the development of a condition, including any of one of the conditions described herein, or advancement of a condition in a patient necessitating treatment.

The terms “kit” and “article of manufacture” are used as synonyms.

As used herein, the term “otic agent” or “otic structure modulating agent” or “otic therapeutic agent” or “otic active agent” or “active agent” or “therapeutic agent” refers to compounds that are effective for the treatment of otic disorders, e.g., otitis media, otosclerosis, autoimmune diseases of the ear and cancer of the ear, and are suitable for use in the formulations disclosed herein. An “otic agent” or “otic structure modulating agent” or “otic therapeutic agent” or “otic active agent” or “active agent” includes, but is not limited to, compounds that act as an agonist, a partial agonist, an antagonist, a partial antagonist, an inverse agonist, a competitive antagonist, a neutral antagonist, an orthosteric antagonist, an allosteric antagonist, a positive allosteric modulator of an otic structure modulating target, a negative allosteric modulator of an otic structure modulating target, or combinations thereof.

The term “otic intervention” means an external insult or trauma to one or more auris structures and includes implants, otic surgery, injections, cannulations, or the like. Implants include auris-interna or auris-media medical devices, examples of which include cochlear implants, hearing sparing devices, hearing-improvement devices, short electrodes, micro-prostheses or piston-like prostheses; needles; stem cell transplants; drug delivery devices; any cell-based therapeutic; or the like. Otic surgery includes middle ear surgery, inner ear surgery, tympanostomy, cochleostomy, labyrinthotomy, mastoidectomy, stapedectomy, stapedotomy, endolymphatic sacculotomy, or the like. Injections include intratympanic injections, intracochlear injections, injections across the round window membrane or the like. Cannulations include intratympanic, intracochlear, endolymphatic, perilymphatic or vestibular cannulations, or the like.

“Pharmacodynamics” refers to the factors which determine the biologic response observed relative to the concentration of drug at the desired site within the auris media and/or auris interna.

“Pharmacokinetics” refers to the factors which determine the attainment and maintenance of the appropriate concentration of drug at the desired site within the auris media and/or auris interna.

In prophylactic applications, compositions containing the otic agents described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like.

As used herein, the term “subject” is used to mean an animal, preferably a mammal, including a human or non-human. The terms patient and subject are used interchangeably.

The terms “treat,” “treating” or “treatment,” as used herein, include alleviating, abating or ameliorating a disease or condition or the associated symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or controlling or stopping the symptoms of the disease or condition either prophylactically and/or therapeutically.

Other objects, features, and advantages of the methods and compositions described herein will become apparent from the following detailed description. It should be understood, however, that the detailed description and the specific examples, while indicating specific embodiments, are given by way of illustration only.

### Anatomy of the Ear

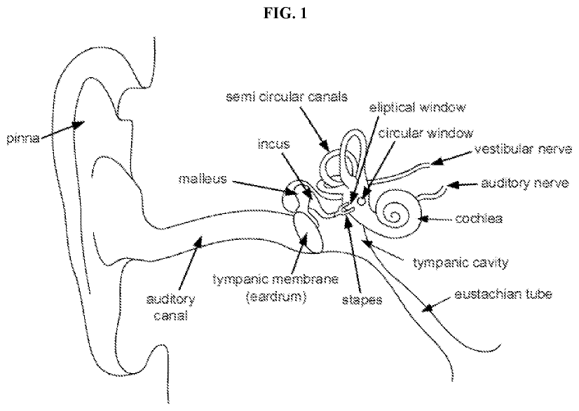

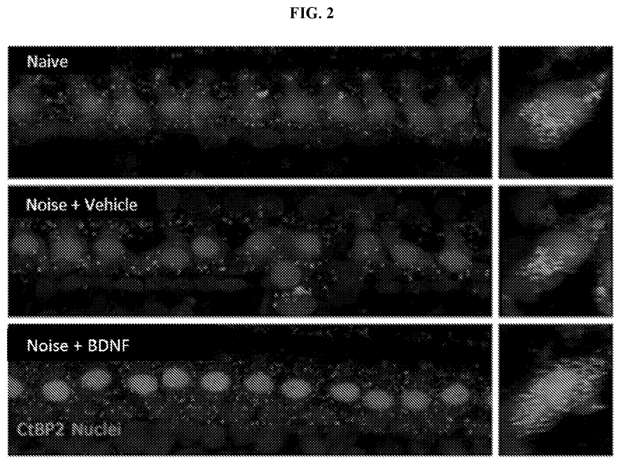

The ear serves as both the sense organ that detects sound and the organ that maintains balance and body position. The ear is generally divided into three portions: the outer ear, middle ear and the inner ear (or auris interna). As shown in FIG. 1, the outer ear is the external portion of the organ and is composed of the pinna (auricle), the auditory canal (external auditory meatus) and the outward facing portion of the tympanic membrane, also known as the ear drum. The pinna, which is the fleshy part of the externa ear that is visible on the side of the head, collects sound waves and directs them toward the auditory canal. Thus, the function of the outer ear, in part, is to collect and direct sound waves towards the tympanic membrane and the middle ear.

The middle ear is an air-filled cavity, called the tympanic cavity, behind the tympanic membrane. The tympanic membrane, also known as the ear drum, is a thin membrane that separates the external ear from the middle ear. The middle ear lies within the temporal bone, and includes within this space the three ear bones (auditory ossicles): the malleus, the incus and the stapes. The auditory ossicles are linked together via tiny ligaments, which form a bridge across the space of the tympanic cavity. The malleus, which is attached to the tympanic membrane at one end, is linked to the incus at its anterior end, which in turn is linked to the stapes. The stapes is attached to the oval window, one of two windows located within the tympanic cavity. A fibrous tissue layer, known as the annular ligament connects the stapes to the oval window. Sound waves from the outer ear first cause the tympanic membrane to vibrate. The vibration is transmitted across to the cochlea through the auditory ossicles and oval window, which transfers the motion to the fluids in the auris interna. Thus, the auditory ossicles are arranged to provide a mechanical linkage between the tympanic membrane and the oval window of the fluid-filled auris interna, where sound is transformed and transduced to the auris interna for further processing. Stiffness, rigidity or loss of movement of the auditory ossicles, tympanic membrane or oval window leads to hearing loss, e.g. otosclerosis, or rigidity of the stapes bone.

The tympanic cavity also connects to the throat via the eustachian tube. The eustachian tube provides the ability to equalize the pressure between the outside air and the middle ear cavity. The round window, a component of the auris interna but which is also accessible within the tympanic cavity, opens into the cochlea of the auris interna. The round window is covered by a membrane, which consists of three layers: an external or mucous layer, an intermediate or fibrous layer, and an internal membrane, which communicates directly with the cochlear fluid. The round window, therefore, has direct communication with the auris interna via the internal membrane.

Movements in the oval and round window are interconnected, i.e. as the stapes bone transmits movement from the tympanic membrane to the oval window to move inward against the auris interna fluid, the round window is correspondingly pushed out and away from the cochlear fluid. This movement of the round window allows movement of fluid within the cochlea, which eventually leads in turn to movement of the cochlear inner hair cells, allowing hearing signals to be transduced. Stiffness and rigidity in the round window leads to hearing loss because of the lack of ability of movement in the cochlear fluid. Recent studies have focused on implanting mechanical transducers onto the round window, which bypasses the normal conductive pathway through the oval window and provides amplified input into the cochlear chamber.

Auditory signal transduction takes place in the auris interna. The fluid-filled inner ear, or auris interna, consists of two major components: the cochlear and the vestibular apparatus.

The cochlea is the portion of the auris interna related to hearing. The cochlea is a tapered tube-like structure which is coiled into a shape resembling a snail. The inside of the cochlea is divided into three regions, which is further defined by the position of the vestibular membrane and the basilar membrane. The portion above the vestibular membrane is the scala vestibuli, which extends from the oval window to the apex of the cochlea and contains perilymph fluid, an aqueous liquid low in potassium and high in sodium content. The basilar membrane defines the scala tympani region, which extends from the apex of the cochlea to the round window and also contains perilymph. The basilar membrane contains thousands of stiff fibers, which gradually increase in length from the round window to the apex of the cochlea. The fibers of the basement membrane vibrate when activated by sound. In between the scala vestibuli and the scala tympani is the cochlear duct, which ends as a closed sac at the apex of the cochlea. The cochlear duct contains endolymph fluid, which is similar to cerebrospinal fluid and is high in potassium.

The Organ of Corti, the sensory organ for hearing, is located on the basilar membrane and extends upward into the cochlear duct. The Organ of Corti contains hair cells, which have hairlike projections that extend from their free surface, and contacts a gelatinous surface called the tectorial membrane. Although hair cells have no axons, they are surrounded by sensory nerve fibers that form the cochlear branch of the vestibulocochlear nerve (cranial nerve VIII).

As discussed, the oval window, also known as the elliptical window communicates with the stapes to relay sound waves that vibrate from the tympanic membrane. Vibrations transferred to the oval window increases pressure inside the fluid-filled cochlea via the perilymph and scala vestibuli/scala tympani, which in turn causes the membrane on the round window to expand in response. The concerted inward pressing of the oval window/outward expansion of the round window allows for the movement of fluid within the cochlea without a change of intra-cochlear pressure. However, as vibrations travel through the perilymph in the scala vestibuli, they create corresponding oscillations in the vestibular membrane. These corresponding oscillations travel through the endolymph of the cochlear duct, and transfer to the basilar membrane. When the basilar membrane oscillates, or moves up and down, the Organ of Corti moves along with it. The hair cell receptors in the Organ of Corti then move against the tectorial membrane, causing a mechanical deformation in the tectorial membrane. This mechanical deformation initiates the nerve impulse which travels via the vestibulocochlear nerve to the central nervous system, mechanically transmitting the sound wave received into signals that are subsequently processed by the central nervous system.

The auris interna is located in part within the osseous or bony labyrinth, an intricate series of passages in the temporal bone of the skull. The vestibular apparatus is the organ of balance and consists of the three semi-circular canals and the vestibule. The three semi-circular canals are arranged relative to each other such that movement of the head along the three orthogonal planes in space is detected by the movement of the fluid and subsequent signal processing by the sensory organs of the semi-circular canals, called the crista ampullaris. The crista ampullaris contains hair cells and supporting cells, and is covered by a dome-shaped gelatinous mass called the cupula. The hairs of the hair cells are embedded in the cupula. The semi-circular canals detect dynamic equilibrium, the equilibrium of rotational or angular movements.

When the head turns rapidly, the semicircular canals move with the head, but endolymph fluid located in the membranous semi-circular canals tends to remain stationary. The endolymph fluid pushes against the cupula, which tilts to one side. As the cupula tilts, it bends some of the hairs on the hair cells of the crista ampullaris, which triggers a sensory impulse. Because each semicircular canal is located in a different plane, the corresponding crista ampullaris of each semi-circular canal responds differently to the same movement of the head. This creates a mosaic of impulses that are transmitted to the central nervous system on the vestibular branch of the vestibulocochlear nerve. The central nervous system interprets this information and initiates the appropriate responses to maintain balance. Of importance in the central nervous system is the cerebellum, which mediates the sense of balance and equilibrium.

The vestibule is the central portion of the auris interna and contains mechanoreceptors bearing hair cells that ascertain static equilibrium, or the position of the head relative to gravity. Static equilibrium plays a role when the head is motionless or moving in a straight line. The membranous labyrinth in the vestibule is divided into two sac-like structures, the utricle and the saccule. Each structure in turn contains a small structure called a macula, which is responsible for maintenance of static equilibrium. The macula consists of sensory hair cells, which are embedded in a gelatinous mass (similar to the cupula) that covers the macula. Grains of calcium carbonate, called otoliths, are embedded on the surface of the gelatinous layer.

When the head is in an upright position, the hairs are straight along the macula. When the head tilts, the gelatinous mass and otoliths tilts correspondingly, bending some of the hairs on the hair cells of the macula. This bending action initiates a signal impulse to the central nervous system, which travels via the vestibular branch of the vestibulocochlear nerve, which in turn relays motor impulses to the appropriate muscles to maintain balance.

In some instances, the otic formulations described herein are placed in the outer ear. In some instances, the otic formulations described herein are placed in the middle or inner ear, including the cochlea and vestibular labyrinth: one option is to use a syringe/needle or pump and inject the formulation across the tympanic membrane (the eardrum). In some instances, for cochlear and vestibular labyrinth delivery, one option is to deliver the active ingredient across the round window membrane or even by microinjection directly into the auris interna also known as cochlear microperfusion.

### Diseases or Conditions of the Ear

In some embodiments, the otic formulations and compositions described herein are suitable for the treatment and/or prevention of diseases or conditions associated with the outer, middle, and/or inner ear. In some embodiments, the otic formulations and compositions described herein are suitable for the treatment and/or prevention of diseases or conditions associated with the outer ear. In some embodiments, the otic formulations and compositions described herein are suitable for the treatment and/or prevention of diseases or conditions associated with the middle ear. In some embodiments, the otic formulations and compositions described herein are suitable for the treatment and/or prevention of diseases or conditions associated with the inner ear. In some embodiments, the otic formulations and compositions described herein reduce, reverse and/or ameliorate symptoms of otic diseases or conditions, such as any one of these disclosed herein. These disorders or conditions have many causes, which include but are not limited to, infection, injury, inflammation, tumors, and adverse response to drugs or other chemical agents.

In some embodiments, the otic formulations and compositions described herein is useful for treating treating an otic disease or condition associated with a synapses-affecting event. In some embodiments, the synapses-affecting event is selected from the group consisting of: head trauma, traumatic brain injury (TBI), acoustic trauma, cochlear implant surgery, sudden sensorineural hearing loss, and drug-induced ototoxicity.

In some embodiments, the drug-induced ototoxicity is chemotherapy-induced ototoxicity. In some embodiments, the drug-induced ototoxicity is cisplatin-induced ototoxicity.

In some embodiments, the drug-induced ototoxicity is antibiotic-induced ototoxicity. In some embodiments, the antibiotic is selected from the group consisting of aminoglycoside antibiotics, macrolide antibiotics, and combinations thereof.

### Hearing Loss

Hearing loss is a partial or total impairment to hearing. Hearing loss is classified into three types, conductive hearing loss, sensorineural hearing loss, and mixed hearing loss. Conductive hearing loss occurs when sound is not conducted efficiently through the external auditory canal to the tympanic membrane or eardrum. In some embodiments, conductive hearing loss involves a reduction in sound level or the ability to hear faint sounds. Treatment involves corrective medical or surgical procedures. Sensorineural hearing loss occurs when there is damage to the cochlea (inner ear), or to the nerve pathways from the cochlea to the brain. This type of hearing loss generally leads to permanent hearing loss. Mixed hearing loss is a combination of conductive hearing loss and sensorineural hearing loss in which damage occurs along both the outer and inner ear regions.

The degree or severity of hearing loss is categorized into seven groups ranging from normal, slight, mild, moderate, moderately severe, severe to profound. In addition, hearing loss is stratified based on frequency in some instances. For example, a hearing loss that only affects the high tones is referred to as a high frequency hearing loss, whereas that which affects the low tones is referred to as a low frequency hearing loss. In some cases, hearing loss affects both high and low frequencies.

Hearing loss is often accompanied by additional causes and symptoms such as ceruminosis, otitis externa, otalgia, tinnitus and vertigo. In some embodiments, it has been shown that ceruminosis can decrease hearing acuity by 40-45 dB. Such impairment, especially in the geriatric population can cause difficulties in communication and even physical immobility. In some embodiments, the otic compositions and formulations disclosed herein are useful for the treatment of hearing loss.

### Hair Cell Regeneration

Hair cells in the mammalian cochlea are important for hearing. The inner and outer hair cells in the Organ of Corti sense vibrations in cochlea fluid produced by sound and transduce these into auditory nerve responses that travel to the brain for sound to be perceived. Loss of hair cells has been implicated hearing loss caused by age, exposure to loud noise, ototoxic drugs, and genetic factors. In birds and amphibians, damage to hair cells triggers mechanisms that cause epithelial cells (supporting cells) in the cochlea to transdifferentiate into new hair cells and to divide and regenerate new supporting cells and hair cells to restore hearing. This ability to regenerate hair cells has been lost in mammals.

In some embodiments, the otic formulations or compositions described herein are useful for the regeneration of otic hair cells.

### Noise Induced Hearing Loss

Noise induced hearing loss (NIHL) is caused upon exposure to sounds that are too loud or loud sounds that last a long time. In some instances, hearing loss occurs from prolonged exposure to loud noises, such as loud music, heavy equipment or machinery, airplanes, or gunfire. Long or repeated or impulse exposure to sounds at or above 85 decibels cause hearing loss in some cases. NIHL causes damage to the hair cells and/or the auditory nerve. The hair cells are small sensory cells that convert sound energy into electrical signals that travel to the brain. In some cases, impulse sound results in immediate hearing loss that is permanent. This kind of hearing loss are accompanied by tinnitus—a ringing, buzzing, or roaring in the ears or head—which subsides over time in some cases. Hearing loss and tinnitus are experienced in one or both ears, and tinnitus continue constantly or occasionally throughout a lifetime in some instances. Permanent damage to hearing loss is often diagnosed. Continuous exposure to loud noise also damages the structure of hair cells, resulting in hearing loss and tinnitus, although the process occurs more gradually than for impulse noise.

In some embodiments, an otoprotectant reverses, reduces, or ameliorates NIHL. Examples of otoprotectants that treat or prevent NIHL include, but are not limited to, D-methionine, L-methionine, ethionine, hydroxyl methionine, methioninol, amifostine, mesna (sodium 2-sulfanylethanesulfonate), a mixture of D and L methionine, normethionine, homomethionine, S-adenosyl-L-methionine), diethyldithiocarbamate, ebselen (2-phenyl-1, 2-benzisoselenazol-3(2H)-one), sodium thiosulfate, AM-111 (a cell permeable JNK inhibitor, (Laboratoires Auris SAS)), leucovorin, leucovorin calcium, dexrazoxane, or combinations thereof.

### Ototoxicity

Ototoxicity refers to hearing loss caused by a toxin. The hearing loss is due to trauma to otic hair cells, the cochlea, and/or the cranial nerve VIII. Multiple drugs are known to be ototoxic. Often ototoxicity is dose-dependent. It is permanent or reversible upon withdrawal of the drug.

Known ototoxic drugs include, but are not limited to, the aminoglycoside class of antibiotics (e.g., gentamicin, and amikacin), some members of the macrolide class of antibiotics (e.g., erythromycin), some members of the glycopeptide class of antibiotics (e.g., vancomycin), salicylic acid, nicotine, some chemotherapeutic agents (e.g., actinomycin, bleomycin, cisplatin, carboplatin and vincristine), and some members of the loop diuretic family of drugs (e.g., furosemide).

Cisplatin and the aminoglycoside class of antibiotics induce the production of reactive oxygen species (“ROS”). ROS damages cells directly by damaging DNA, polypeptides, and/or lipids. Antioxidants prevent damage of ROS by preventing their formation or scavenging free radicals before they damage the cell. Both cisplatin and the aminoglycoside class of antibiotics are also thought to damage the ear by binding melanin in the stria vascularis of the inner ear.

Salicylic acid is classified as ototoxic as it inhibits the function of the polypeptide prestin. Prestin mediates outer otic hair cell motility by controlling the exchange of chloride and carbonate across the plasma membrane of outer otic hair cells. It is only found in the outer otic hair cells, not the inner otic hair cells. Accordingly, in some embodiments, the use of the controlled release auris-compositions described herein, ameliorates or lessens ototoxic effects of chemotherapy, including but not limited to cisplatin treatment, aminoglycoside or salicylic acid administration, or other ototoxic agents.

### Drug-Induced Inner Ear Damage

Damage to all or a portion of the ear (e.g., inner ear) may be caused by the administration of drugs, including certain antibiotics, diuretics (e.g. ethacrynic acid and furosemide), aspirin, aspirin-like substances (e.g. salicylates) and quinine Deterioration of the auris interna organ are hastened by impaired kidney function, which results in decreased clearance of the affecting drugs and their metabolites. In some instances, these drugs affect both hearing and equilibrium; however, these drugs likely affect hearing to a greater extent.

For example, neomycin, kanamycin, amikacin have a greater effect on hearing than on balance. The antibiotics viomycin, gentamicin and tobramycin affect both hearing and equilibrium. Streptomycin, another commonly administered antibiotic, induces vertigo more than loss of hearing, and leads to Dandy's syndrome, where walking in the dark becomes difficult and induces a sensation of the environment moving with each step. Aspirin, when taken in very high doses, also leads to temporary hearing loss and tinnitus, a condition where sound is perceived in the absence of external sound. Similarly, quinine, ethacryinic acid and furosemide result in temporary or permanent hearing loss in some instances.

### Sudden Sensorineural Hearing Loss

Sudden sensorineural hearing loss (SSNHL) is most often defined as sensorineural hearing loss of 30 dB or greater over at least three contiguous audiometric frequencies occurring over 72 hr (Wilson, Byl, & Laird, 1980). SSNHL is a relatively common complaint in otologic and audiologic practices (1.5-1.7 per 100 new patients presenting in our practice). For 7% to 45% of patients, a defined cause can be identified and specific therapeutic regiment used for treatment (Byl, 1984; Chau, Lin, Atashband, Irvine, & Westerberg, 2010; Fetterman, Saunders, & Luxford, 1996; Huy & Sauvaget, 2005; Nosrati-Zarenoe, Arlinger, & Hultcrantz, 2007; Shaia & Sheehy, 1976). The majority of patients with sudden SNHL have no identifiable cause for hearing loss and are classified as “idiopathic” (Byl, 1984; Chau et al., 2010; Fetterman et al., 1996; Nosrati-Zarenoe et al., 2007; Shaia & Sheehy, 1976). Despite extensive research, controversy remains in the etiology and appropriate care of patients with idiopathic SSNHL Regardless of etiology, recovery of hearing thresholds following SSNHL may not occur, may be partial, or can be complete. Factors impacting hearing recovery include age at onset of hearing loss, hearing loss severity and frequencies affected, presence of vertigo, and time between onset of hearing loss and visit with the treating physician (Byl, 1984).

### Temporal Bone Fractures

The temporal bone, which contains part of the ear canal, the middle ear and the auris interna, is subject to fractures from blows to the skull or other injuries. Bleeding from the ear or patchy bruising is symptomatic of a fracture to the temporal bone, and requires a computed tomography (CT) scan for accurate diagnosis. Temporal bone fractures rupture the eardrum, causing facial paralysis and sensorineural hearing loss.

Treatment of detected temporal bone fractures includes an antibiotic regimen to prevent meningitis, or an infection of brain tissue. In addition, surgery is performed to relieve any subsequent pressure on the facial nerve due to swelling or infection.

### Local Otic Administration

Also provided herein are methods, formulations, and compositions for local delivery of therapeutic agents (otic agents) to auris externa, auris media, and/or auris interna structures. In some embodiments, local delivery of the therapeutic agent (otic agent) overcomes the toxic and attendant side effects of systemic delivery. In some embodiments, access to the vestibular and cochlear apparatus is through the auris media and includes the round window membrane, the oval window/stapes footplate, the annular ligament and through the otic capsule/temporal bone.

Provided herein, in certain embodiments, are otic formulations and compositions that remain in contact with the target auditory surfaces (e.g., the round window) for extended periods of time. In some embodiments, the otic formulations and compositions further comprise mucoadhesives that allow the otic formulations to adhere to otic mucosal surfaces. In some instances, the formulations and compositions described herein avoid attenuation of therapeutic benefit due to drainage or leakage of active agents via the eustachian tube.

In some embodiments, the localized treatment of the auris externa, auris media and/or auris interna affords the use of previously undesired therapeutic agents, including agents with poor PK profiles, poor uptake, low systemic release and/or toxicity issues. In some embodiments, localized targeting of the otic agent formulations and compositions reduces the risk of adverse effects with previously characterized toxic or ineffective therapeutic agents (otic active agents). Accordingly, also contemplated within the scope of the embodiments described herein is the use of active agents and/or agents that have been previously rejected by practitioners because of adverse effects or ineffectiveness of the therapeutic agent (otic agent).

In some embodiments, specifically targeting the auris externa, auris media and/or auris interna structures avoids the adverse side effects usually associated with systemic treatment. In some embodiments, the otic formulations and compositions described herein are controlled release therapeutic agent formulations and compositions that treat otic disorders by providing a constant, variable and/or extended source of a therapeutic agent (otic agent) to the individual or patient suffering from an otic disorder, thereby reducing or eliminating the variability of treatment. Accordingly, one embodiment disclosed herein is to provide a formulation or composition that enables at least one therapeutic agent (otic agent) to be released in therapeutically effective doses either at variable or constant rates such as to ensure a continuous release of the at least one therapeutic agent (otic agent). In some embodiments, the therapeutic agents (otic agents) disclosed herein are administered as an immediate release formulation or composition. In other embodiments, the therapeutic agents (otic agents) are administered as a controlled release formulation, released either continuously or in a pulsatile manner, or variants of both. In still other embodiments, the therapeutic agent (otic agent) formulation or composition is administered as both an immediate release and controlled release formulation or composition, released either continuously or in a pulsatile manner, or variants of both. The release is optionally dependent on environmental or physiological conditions, for example, the external ionic environment (see, e.g. Oros® release system, Johnson & Johnson).

In addition, the otic compositions or formulations included herein also optionally include carriers, adjuvants, such as preserving, stabilizing, wetting or emulsifying agents, solution promoters, salts for regulating the osmotic pressure, and/or buffers. Such carriers, adjuvants, and other excipients are compatible with the environment in the auris externa, auris media and/or auris interna. Accordingly, specifically contemplated are carriers, adjuvants and excipients that lack ototoxicity or are minimally ototoxic in order to allow effective treatment of the otic disorders contemplated herein with minimal side effects in the targeted regions or areas. To prevent ototoxicity, otic compositions or formulations disclosed herein are optionally targeted to distinct regions of the auris externa, auris media and/or auris interna, including but not limited to the tympanic cavity, vestibular bony and membranous labyrinths, cochlear bony and membranous labyrinths and other anatomical or physiological structures located within the auris interna.

### Treatment

Provided herein are otic formulations and compositions suitable for the treatment of any otic condition, disease or disorder (e.g., outer, middle and/or inner ear disorders) described herein, comprising administration of a therapeutic agent (otic agent) described herein to an individual or patient in need thereof. The formulations and compositions described herein are suitable for the treatment of any disease described herein. In some instances, the treatment is long-term treatment for chronic recurring disease. In some instances, the treatment is prophylactic administration of an otic formulation described herein for the treatment of any otic disease or disorder described herein. In some instances, prophylactic administration avoids occurrence of disease in individuals suspected of having a disease or in individuals genetically predisposed to an otic disease or disorder. In some instances the treatment is preventive maintenance therapy. In some instances, preventive maintenance therapy avoids recurrence of a disease.

In some instances, because of their otic compatibility and improved sterility, the otic formulations and compositions described herein are safe for long-term administration. In some embodiments, the otic formulations and compositions described herein have very low ototoxicity.

In some embodiments, the otic formulations and compositions described herein provide a steady sustained release of a therapeutic agent (otic agent) for a period of at least one day, three days, five days, one week, two weeks, three weeks, a month, two months, three months, four months, five months, six months, or a year. In some embodiments, the otic formulations and compositions described herein provide a steady sustained release of a therapeutic agent (otic agent) for a period of at least three days. In some embodiments, the otic formulations and compositions described herein provide a steady sustained release of a therapeutic agent (otic agent) for a period of at least five days. In some embodiments, the otic formulations and compositions described herein provide a steady sustained release of a therapeutic agent (otic agent) for a period of at least one week. In some embodiments, the otic formulations and compositions described herein provide a steady sustained release of a therapeutic agent (otic agent) for a period of at least two weeks. In some embodiments, the otic formulations and compositions described herein provide a steady sustained release of a therapeutic agent (otic agent) for a period of at least three weeks. In some embodiments, the otic formulations and compositions described herein provide a steady sustained release of a therapeutic agent (otic agent) for a period of at least a month. In some embodiments, the otic formulations and compositions described herein provide a steady sustained release of a therapeutic agent (otic agent) for a period of at least two months. In some embodiments, the otic formulations and compositions described herein provide a steady sustained release of a therapeutic agent (otic agent) for a period of at least three months. In some embodiments, the otic formulations and compositions described herein provide a steady sustained release of a therapeutic agent (otic agent) for a period of at least four months. In some embodiments, the otic formulations and compositions described herein provide a steady sustained release of a therapeutic agent (otic agent) for a period of at least five months. In some embodiments, the otic formulations and compositions described herein provide a steady sustained release of a therapeutic agent (otic agent) for a period of at least six months. In some embodiments, the otic formulations and compositions described herein provide a steady sustained release of a therapeutic agent (otic agent) for a period of at least a year.

In some embodiments, the otic formulations and compositions described herein provide a steady sustained release of a therapeutic agent (otic agent) for a period of about a day, three days, five days, one week, two weeks, three weeks, a month, two months, three months, four months, five months, six months, or a year. In some embodiments, the otic formulations and compositions described herein provide a steady sustained release of a therapeutic agent (otic agent) for a period of about three days. In some embodiments, the otic formulations and compositions described herein provide a steady sustained release of a therapeutic agent (otic agent) for a period of about five days. In some embodiments, the otic formulations and compositions described herein provide a steady sustained release of a therapeutic agent (otic agent) for a period of about one week. In some embodiments, the otic formulations and compositions described herein provide a steady sustained release of a therapeutic agent for a period of about two weeks. In some embodiments, the otic formulations and compositions described herein provide a steady sustained release of a therapeutic agent (otic agent) for a period of about three weeks. In some embodiments, the otic formulations and compositions described herein provide a steady sustained release of a therapeutic agent (otic agent) for a period of about a month. In some embodiments, the otic formulations and compositions described herein provide a steady sustained release of a therapeutic agent (otic agent) for a period of about two months. In some embodiments, the otic formulations and compositions described herein provide a steady sustained release of a therapeutic agent (otic agent) for a period of about three months. In some embodiments, the otic formulations and compositions described herein provide a steady sustained release of a therapeutic agent (otic agent) for a period of about four months. In some embodiments, the otic formulations and compositions described herein provide a steady sustained release of a therapeutic agent (otic agent) for a period of about five months. In some embodiments, the otic formulations and compositions described herein provide a steady sustained release of a therapeutic agent (otic agent) for a period of about six months. In some embodiments, the otic formulations and compositions described herein provide a steady sustained release of a therapeutic agent (otic agent) for a period of about a year.

### Timing of Administration

Provided herein are methods of treating an otic disease or condition (e.g. hearing loss of hearing impairment) associated with a synapses-affecting event in a subject in need thereof. According to one aspect, the method comprises administering an otic formulation comprising a therapeutically effective amount of a growth factor and an auris-acceptable vehicle. In some instances, the otic formulation is formulated to provide sustained release of the growth factor into the inner ear to promote formation of synapses or repaire damaged synapses. In some cases, the otic formulation is administered within a period of time after onset of the synapses-affecting event such as at least a portion of the affected synapses can repaired and/or restored. In some cases, when the otic formulation is administered after expiration of a period of time after onset of the synapses-affecting event, the affected synapses cannot be effectively repaired or restored by the growth factor in the otic formulation.

In some embodiments, the otic formulation is administered within 12 months after onset of the synapses-damaging event. In some embodiments, the otic formulation is administered within 11 months after onset of the synapses-damaging event. In some embodiments, the otic formulation is administered within 10 months after onset of the synapses-damaging event. In some embodiments, the otic formulation is administered within 9 months after onset of the synapses-damaging event. In some embodiments, the otic formulation is administered within 8 months after onset of the synapses-damaging event. In some embodiments, the otic formulation is administered within 7 months after onset of the synapses-damaging event. In some embodiments, the otic formulation is administered within 6 months after onset of the synapses-damaging event. In some embodiments, the otic formulation is administered within 5 months after onset of the synapses-damaging event. In some embodiments, the otic formulation is administered within 4 months after onset of the synapses-damaging event. In some embodiments, the otic formulation is administered within 3 months after onset of the synapses-damaging event. In some embodiments, the otic formulation is administered within 2 months after onset of the synapses-damaging event. In some embodiments, the otic formulation is administered within 1 month after onset of the synapses-damaging event.

In some embodiments, the otic formulation is administered within 30 days after onset of the synapses-damaging event. In some embodiments, the otic formulation is administered within 25 days after onset of the synapses-damaging event. In some embodiments, the otic formulation is administered within 20 days after onset of the synapses-damaging event. In some embodiments, the otic formulation is administered within 15 days after onset of the synapses-damaging event. In some embodiments, the otic formulation is administered within 14 days after onset of the synapses-damaging event. In some embodiments, the otic formulation is administered within 13 days after onset of the synapses-damaging event. In some embodiments, the otic formulation is administered within 12 days after onset of the synapses-damaging event. In some embodiments, the otic formulation is administered within 11 days after onset of the synapses-damaging event. In some embodiments, the otic formulation is administered within 10 days after onset of the synapses-damaging event. In some embodiments, the otic formulation is administered within 9 days after onset of the synapses-damaging event. In some embodiments, the otic formulation is administered within 8 days after onset of the synapses-damaging event. In some embodiments, the otic formulation is administered within 7 days after onset of the synapses-damaging event. In some embodiments, the otic formulation is administered within 6 days after onset of the synapses-damaging event. In some embodiments, the otic formulation is administered within 5 days after onset of the synapses-damaging event. In some embodiments, the otic formulation is administered within 4 days after onset of the synapses-damaging event. In some embodiments, the otic formulation is administered within 3 days after onset of the synapses-damaging event. In some embodiments, the otic formulation is administered within 2 days after onset of the synapses-damaging event. In some embodiments, the otic formulation is administered within 1 day after onset of the synapses-damaging event.

In some embodiments, the otic formulation is administered within 22 hours after onset of the synapses-damaging event. In some embodiments, the otic formulation is administered within 20 hours after onset of the synapses-damaging event. In some embodiments, the otic formulation is administered within 18 hours after onset of the synapses-damaging event. In some embodiments, the otic formulation is administered within 16 hours after onset of the synapses-damaging event. In some embodiments, the otic formulation is administered within 14 hours after onset of the synapses-damaging event. In some embodiments, the otic formulation is administered within 12 hours after onset of the synapses-damaging event. In some embodiments, the otic formulation is administered within 11 hours after onset of the synapses-damaging event. In some embodiments, the otic formulation is administered within 10 hours after onset of the synapses-damaging event. In some embodiments, the otic formulation is administered within 9 hours after onset of the synapses-damaging event. In some embodiments, the otic formulation is administered within 8 hours after onset of the synapses-damaging event. In some embodiments, the otic formulation is administered within 7 hours after onset of the synapses-damaging event. In some embodiments, the otic formulation is administered within 6 hours after onset of the synapses-damaging event. In some embodiments, the otic formulation is administered within 5 hours after onset of the synapses-damaging event. In some embodiments, the otic formulation is administered within 4 hours after onset of the synapses-damaging event. In some embodiments, the otic formulation is administered within 3 hours after onset of the synapses-damaging event. In some embodiments, the otic formulation is administered within 2 hours after onset of the synapses-damaging event. In some embodiments, the otic formulation is administered within 1 hour after onset of the synapses-damaging event.

### Therapeutic Agents

In some embodiments, the otic formulations and compositions described herein have pH and osmolarity that are auris-acceptable. In some embodiments, the otic formulations and compositions described herein meet the stringent sterility requirements described herein and are compatible with the endolymph and/or the perilymph. Pharmaceutical agents that are used in conjunction with the formulations and compositions disclosed herein include agents that ameliorate or lessen otic disorders, including auris interna disorders, and their attendant symptoms, which include but are not limited to hearing loss, nystagmus, vertigo, tinnitus, inflammation, swelling, infection and congestion. Otic disorders have many causes and include infection, injury, inflammation, tumors and adverse response to drugs or other chemical agents that are responsive to the pharmaceutical agents disclosed herein. In some embodiments, pharmaceutically active metabolites, salts, polymorphs, prodrugs, analogues, and derivatives of the otic agents disclosed herein are used in the formulations.

For some embodiments, wherein the formulation or composition is designed such that the active ingredient has limited or no systemic release, therapeutic agents that produce systemic toxicities (e.g., liver toxicity) or have poor PK characteristics (e.g. short half-life) are also optionally used. Thus, in some embodiments, therapeutic agents that have been previously shown to be toxic, harmful or non-effective during systemic application, for example through toxic metabolites formed after hepatic processing, toxicity of the drug in particular organs, tissues or systems, through high levels needed to achieve efficacy, through the inability to be released through systemic pathways or through poor PK characteristics, are useful. The formulations and compositions disclosed herein are contemplated to be targeted directly to otic structures where treatment is needed; for example, one embodiment contemplated is the direct application of the otic formulations disclosed herein onto the round window membrane or the crista fenestrae cochlea of the auris interna, allowing direct access and treatment of the auris interna, or inner ear components. In other embodiments, the otic formulations and compositions disclosed herein are applied directly to the oval window. In yet other embodiments, direct access is obtained through microinjection directly into the auris interna, for example, through cochlear microperfusion. Such embodiments also optionally comprise a drug delivery device, wherein the drug delivery device delivers the otic formulations through use of a needle and syringe, a pump, a microinjection device, a spongy material or any combination thereof.

In still other embodiments, application of any otic formulation or composition described herein is targeted to the auris media through piercing of the intratympanic membrane and applying the otic agent formulation directly to the auris media structures affected, including the walls of the tympanic cavity or auditory ossicles. In some embodiments, the auris active agent formulations and compositions disclosed herein are confined to the targeted auris media structure, and will not be lost, for example, through diffusion or leakage through the eustachian tube or pierced tympanic membrane. In some embodiments, the otic formulations and compositions disclosed herein are delivered to the auris externa in any suitable manner, including by cotton swab, injection or ear drops. Also, in other embodiments, the otic formulations and compositions described herein are targeted to specific regions of the auris externa by application with a needle and syringe, a pump, a microinjection device, a spongy material, or any combination thereof. For example, in the case of treatment of otitis externa, antimicrobial agent formulations disclosed herein are delivered directly to the ear canal, where they are retained, thereby reducing loss of the active agents from the target ear structure by drainage or leakage.

### Growth Factors

Contemplated for use with the formulations disclosed herein are agents that modulate the degeneration of neurons and/or hair cells of the auris, promote the survival and/or growth of neurons and/or hair cells of the auris, and agents for treating or ameliorating hearing loss or reduction resulting from destroyed, stunted, malfunctioning, damaged, fragile or missing hairs in the inner ear. Accordingly, some embodiments incorporate the use of agents which promote the survival of neurons and otic hair cells, and/or the growth of neurons and otic hair cells. In some embodiments, the agent which promotes the survival of otic hair cells is a growth factor. In some embodiments, the growth factor is a neurotroph. In certain instances, neurotrophs are growth factors which prevent cells from initiating apoptosis, repair damaged neurons and otic hair cells, and/or induce differentiation in progenitor cells. In some embodiments, the growth factor is brain-derived neurotrophic factor (BDNF), ciliary neurotrophic factor (CNTF), glial cell-line derived neurotrophic factor (GDNF), neurotrophin-3, neurotrophin-4, and/or combinations thereof. In some embodiments, the growth factor is brain-derived neurotrophic factor (BDNF). In some embodiments, the growth factor is ciliary neurotrophic factor (CNTF). In some embodiments, the growth factor is glial cell-line derived neurotrophic factor (GDNF). In some embodiments, the growth factor is neurotrophin-3. In some embodiments, the growth factor is neurotrophin-4. In some embodiments, the growth factor is a fibroblast growth factor (FGF), an insulin-like growth factor (IGF), an epidermal growth factor (EGF), a platelet-derived growth factor (PGF) and/or agonists thereof. In some embodiments, the growth factor is an agonist of the fibroblast growth factor (FGF) receptor, the insulin-like growth factor (IGF) receptor, the epidermal growth factor (EGF) receptor, and/or the platlet-derived growth factor. In some embodiments, the growth factor is hepatocyte growth factor.

In some embodiments, the growth factor is an epidermal growth factor (EGF). In some embodiments, the EGF is heregulin (HRG). In certain instances, HRG stimulates the proliferation of utricular sensory epithelium. In certain instances, HRG-binding receptors are found in the vestibular and auditory sensory epithelium.

In some embodiments, the growth factor is an insulin-like growth factor (IGF). In some embodiments, the IGF is IGF-1. In some embodiments, the IGF-1 is mecasermin. In certain instances, IGF-1 attenuates the damage induced by exposure to an aminoglycoside. In certain instances, IGF-1 stimulates the differentiation and/or maturation of cochlear ganglion cells.

In some embodiments, the FGF receptor agonist is FGF-2. In some embodiments, the IGF receptor agonist is IGF-1. Both the FGF and IGF receptors are found in the cells comprising the utricle epithelium.

In some embodiments, the growth factor is hepatocyte growth factor (HGF). In some instances, HGF protects cochlear hair cells from noise-induced damage and reduces noise-exposure-caused ABR threshold shifts.

Also contemplated for use in the otic formulations described herein are growth factors including Erythropoietin (EPO), Granulocyte-colony stimulating factor (G-CSF), Granulocyte-macrophage colony stimulating factor (GM-CSF), Growth differentiation factor-9 (GDF9), Insulin-like growth factor (IGF), Myostatin (GDF-8), Platelet-derived growth factor (PDGF), Thrombopoietin (TPO), Transforming growth factor alpha (TGF-α), Transforming growth factor beta (TGF-β), Vascular endothelial growth factor (VEGF) or combinations thereof.

### Neurotrophs

In some embodiments, the growth factor is a neurotroph. In certain instances, neurotrophs are growth factors which prevent cells from initiating apoptosis, repair damaged neurons and otic hair cells, and/or induce differentiation in progenitor cells. In some embodiments, the neurotroph is brain-derived neurotrophic factor (BDNF), ciliary neurotrophic factor (CNTF), glial cell-line derived neurotrophic factor (GDNF), neurotrophin-3, neurotrophin-4, and/or combinations thereof.

In some embodiments, the neurotroph is BDNF. In certain instances, BDNF is a neurotroph which promotes the survival of existing neurons (e.g. spiral ganglion neurons), and otic hair cells by repairing damaged cells, inhibiting the production of ROS, and inhibiting the induction of apoptosis. In certain embodiments, it also promotes the differentiation of neural and otic hair cell progenitors. Further, in certain embodiments, it protects the Cranial Nerve VII from degeneration. In some embodiments, BDNF is administered in conjunction with fibroblast growth factor.

In some embodiments, the neurotroph is neurotrophin-3. In certain embodiments, neurotrophin-3 promotes the survival of existing neurons and otic hair cells, and promotes the differentiation of neural and otic hair cell progenitors. Further, in certain embodiments, it protects the VII nerve from degeneration.

In some embodiments, the neurotroph is CNTF. In certain embodiments, CNTF promotes the synthesis of neurotransmitters and the growth of neuritis. In some embodiments, CNTF is administered in conjunction with BDNF.

In some embodiments, the neurotroph is GDNF. In certain embodiments, GDNF expression is increased by treatment with ototoxic agents. Further, in certain embodiments, cells treated with exogenous GDNF have higher survival rates after trauma then untreated cells.

In some embodiments, the neurotroph is a Trk agonist, such as a TrkB or TrkC agonist. For example, BDNF and NT-3 activate TrkB and TrkC on spiral ganglion neurons to prompt survival, neurite growth, and synapse formation. Other Trk agonists including TrkC monoclonal antibody agonists M1, M2, and M7 humanized and TrkB monoclonal antibody agonists M3 and M6 humanized and M4 and M5 mouse, as described in WO2017/019907, are also contemplated for use according to the methods provided herein.

### Amount of Therapeutic Agent

In some embodiments, the otic formulation comprises between about 0.0001% to about 99.9999% by weight of the weight of the therapeutic agent (e.g., growth factor), or pharmaceutically acceptable prodrug or salt thereof. In some embodiments, the otic formulation comprises between about 0.0001% to about 20% by weight of the weight of the therapeutic agent, or pharmaceutically acceptable prodrug or salt thereof. In some embodiments, the otic formulation comprises between about 0.0001% to about 15% by weight of the weight of the therapeutic agent, or pharmaceutically acceptable prodrug or salt thereof. In some embodiments, the otic formulation comprises between about about 0.0001% to about 10% by weight of the weight of the therapeutic agent, or pharmaceutically acceptable prodrug or salt thereof. In some embodiments, the otic formulation comprises between about 0.0001% to about 5% by weight of the weight of the therapeutic agent, or pharmaceutically acceptable prodrug or salt thereof. In some embodiments, the otic formulation comprises between about 0.0001% to about 1% by weight of the weight of the therapeutic agent, or pharmaceutically acceptable prodrug or salt thereof. In some embodiments, the otic formulation comprises between about 0.05% to about 0.5% by weight of the therapeutic agent, or pharmaceutically acceptable prodrug or salt thereof.

In some embodiments, the otic formulation or composition comprises between about 0.001% to about 99.99% by weight of the therapeutic agent, or pharmaceutically acceptable prodrug or salt thereof. In some embodiments, the otic formulation or composition comprises between about 0.001% to about 99.9% by weight of the therapeutic agent, or pharmaceutically acceptable prodrug or salt thereof. In some embodiments, the otic formulation or composition comprises between about 0.001% to about 99% by weight of the therapeutic agent, or pharmaceutically acceptable prodrug or salt thereof. In some embodiments, the otic formulation or composition comprises between about 0.001% to about 90% by weight of the therapeutic agent, or pharmaceutically acceptable prodrug or salt thereof. In some embodiments, the otic formulation or composition comprises between about 0.001% to about 80% by weight of the therapeutic agent, or pharmaceutically acceptable prodrug or salt thereof. In some embodiments, the otic formulation or composition comprises between about 0.001% to about 70% by weight of the therapeutic agent, or pharmaceutically acceptable prodrug or salt thereof. In some embodiments, the otic formulation or composition comprises between about 0.001% to about 60% by weight of the therapeutic agent, or pharmaceutically acceptable prodrug or salt thereof. In some embodiments, the otic formulation or composition comprises between about 0.001% to about 50% by weight of the therapeutic agent, or pharmaceutically acceptable prodrug or salt thereof. In some embodiments, the otic formulation or composition comprises between about 0.001% to about 40% by weight of the therapeutic agent, or pharmaceutically acceptable prodrug or salt thereof. In some embodiments, the otic formulation or composition comprises between about 0.001% to about 30% by weight of the therapeutic agent, or pharmaceutically acceptable prodrug or salt thereof. In some embodiments, the otic formulation or composition comprises between about 0.001% to about 20% by weight of the therapeutic agent, or pharmaceutically acceptable prodrug or salt thereof. In some embodiments, the otic formulation or composition comprises between about 0.001% to about 15% by weight of the therapeutic agent, or pharmaceutically acceptable prodrug or salt thereof. In some embodiments, the otic formulation or composition comprises between about 0.001% to about 10% by weight of the therapeutic agent, or pharmaceutically acceptable prodrug or salt thereof. In some embodiments, the otic formulation or composition comprises between about 0.001% to about 7% by weight of the therapeutic agent, or pharmaceutically acceptable prodrug or salt thereof. In some embodiments, the otic formulation or composition comprises between about 0.001% to about 5% by weight of the therapeutic agent, or pharmaceutically acceptable prodrug or salt thereof. In some embodiments, the otic formulation or composition comprises between about 0.001% to about 3% by weight of the therapeutic agent, or pharmaceutically acceptable prodrug or salt thereof. In some embodiments, the otic formulation or composition comprises between about 0.001% to about 2% by weight of the therapeutic agent, or pharmaceutically acceptable prodrug or salt thereof. In some embodiments, the otic formulation or composition comprises between about 0.001% to about 1% by weight of the therapeutic agent, or pharmaceutically acceptable prodrug or salt thereof. In some embodiments, the otic formulation comprises between about 0.05% to about 0.5% by weight of the therapeutic agent, or pharmaceutically acceptable prodrug or salt thereof.

In some embodiments, the otic formulation or composition comprises between about 0.01% to about 99.99% by weight of the therapeutic agent (e.g., growth factor), or pharmaceutically acceptable prodrug or salt thereof. In some embodiments, the otic formulation or composition comprises between about 0.01% to about 99.9% by weight of the therapeutic agent, or pharmaceutically acceptable prodrug or salt thereof. In some embodiments, the otic formulation or composition comprises between about 0.01% to about 99% by weight of the therapeutic agent, or pharmaceutically acceptable prodrug or salt thereof. In some embodiments, the otic formulation or composition comprises between about 0.01% to about 90% by weight of the therapeutic agent, or pharmaceutically acceptable prodrug or salt thereof. In some embodiments, the otic formulation or composition comprises between about 0.01% to about 80% by weight of the therapeutic agent, or pharmaceutically acceptable prodrug or salt thereof. In some embodiments, the otic formulation or composition comprises between about 0.01% to about 70% by weight of the therapeutic agent, or pharmaceutically acceptable prodrug or salt thereof. In some embodiments, the otic formulation or composition comprises between about 0.01% to about 60% by weight of the therapeutic agent, or pharmaceutically acceptable prodrug or salt thereof. In some embodiments, the otic formulation or composition comprises between about 0.01% to about 50% by weight of the therapeutic agent, or pharmaceutically acceptable prodrug or salt thereof. In some embodiments, the otic formulation or composition comprises between about 0.01% to about 40% by weight of the therapeutic agent, or pharmaceutically acceptable prodrug or salt thereof. In some embodiments, the otic formulation or composition comprises between about 0.01% to about 30% by weight of the therapeutic agent, or pharmaceutically acceptable prodrug or salt thereof. In some embodiments, the otic formulation or composition comprises between about 0.01% to about 20% by weight of the therapeutic agent, or pharmaceutically acceptable prodrug or salt thereof. In some embodiments, the otic formulation or composition comprises between about 0.01% to about 15% by weight of the therapeutic agent, or pharmaceutically acceptable prodrug or salt thereof. In some embodiments, the otic formulation or composition comprises between about 0.01% to about 10% by weight of the therapeutic agent, or pharmaceutically acceptable prodrug or salt thereof. In some embodiments, the otic formulation or composition comprises between about 0.01% to about 7% by weight of the therapeutic agent, or pharmaceutically acceptable prodrug or salt thereof. In some embodiments, the otic formulation or composition comprises between about 0.01% to about 5% by weight of the therapeutic agent, or pharmaceutically acceptable prodrug or salt thereof. In some embodiments, the otic formulation or composition comprises between about 0.01% to about 3% by weight of the therapeutic agent, or pharmaceutically acceptable prodrug or salt thereof. In some embodiments, the otic formulation or composition comprises between about 0.01% to about 2% by weight of the therapeutic agent, or pharmaceutically acceptable prodrug or salt thereof. In some embodiments, the otic formulation or composition comprises between about 0.01% to about 1% by weight of the therapeutic agent, or pharmaceutically acceptable prodrug or salt thereof.

In some embodiments, the otic formulation or composition comprises between about 0.1% to about 40% by weight of the therapeutic agent (e.g., growth factor), or pharmaceutically acceptable prodrug or salt thereof. In some embodiments, the otic formulation or composition comprises between about 0.1% to about 30% by weight of the therapeutic agent, or pharmaceutically acceptable prodrug or salt thereof. In some embodiments, the otic formulation or composition comprises between about 0.10% to about 20% by weight of the therapeutic agent, or pharmaceutically acceptable prodrug or salt thereof. In some embodiments, the otic formulation or composition comprises between about 0.10% to about 15% by weight of the therapeutic agent, or pharmaceutically acceptable prodrug or salt thereof. In some embodiments, the otic formulation or composition comprises between about 0.10% to about 10% by weight of the therapeutic agent, or pharmaceutically acceptable prodrug or salt thereof. In some embodiments, the otic formulation or composition comprises between about 0.10% to about 7% by weight of the therapeutic agent, or pharmaceutically acceptable prodrug or salt thereof. In some embodiments, the otic formulation or composition comprises between about 0.10% to about 5% by weight of the therapeutic agent, or pharmaceutically acceptable prodrug or salt thereof. In some embodiments, the otic formulation or composition comprises between about 0.10% to about 3% by weight of the therapeutic agent, or pharmaceutically acceptable prodrug or salt thereof. In some embodiments, the otic formulation or composition comprises between about 0.10% to about 2% by weight of the therapeutic agent, or pharmaceutically acceptable prodrug or salt thereof. In some embodiments, the otic formulation or composition comprises between about 0.10% to about 1% by weight of the therapeutic agent, or pharmaceutically acceptable prodrug or salt thereof.

In some embodiments, the otic formulation or composition comprises between about 1% to about 40% by weight of the therapeutic agent (e.g., growth factor), or pharmaceutically acceptable prodrug or salt thereof. In some embodiments, the otic formulation or composition comprises between about 1% to about 30% by weight of the therapeutic agent, or pharmaceutically acceptable prodrug or salt thereof. In some embodiments, the otic formulation or composition comprises between about 1% to about 20% by weight of the therapeutic agent, or pharmaceutically acceptable prodrug or salt thereof. In some embodiments, the otic formulation or composition comprises between about 1% to about 15% by weight of the therapeutic agent, or pharmaceutically acceptable prodrug or salt thereof. In some embodiments, the otic formulation or composition comprises between about 1% to about 10% by weight of the therapeutic agent, or pharmaceutically acceptable prodrug or salt thereof. In some embodiments, the otic formulation or composition comprises between about 1% to about 7% by weight of the therapeutic agent, or pharmaceutically acceptable prodrug or salt thereof. In some embodiments, the otic formulation or composition comprises between about 1% to about 5% by weight of the therapeutic agent, or pharmaceutically acceptable prodrug or salt thereof. In some embodiments, the otic formulation or composition comprises between about 1% to about 3% by weight of the therapeutic agent, or pharmaceutically acceptable prodrug or salt thereof. In some embodiments, the otic formulation or composition comprises between about 1% to about 2% by weight of the therapeutic agent, or pharmaceutically acceptable prodrug or salt thereof.

In some embodiments, the otic formulation or composition comprises about 0.0001%, about 0.0002%, about 0.0003%, about 0.0004%, about 0.0005%, about 0.0006%, about 0.0007%, about 0.0008%, about 0.0009%, about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7% m about 8%, about 9%, about 10%, about about 11%, about 12%, about 13%, about 14%, about 15%, about 16%, about 17%, about 18%, about 19%, about 20%, about 25%, about 30%, about 35%, or about 40% by weight of the therapeutic agent (e.g., growth factor), or pharmaceutically acceptable prodrug or salt thereof.

In some embodiments, the otic formulation or composition comprises about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2% about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, or about 40% by weight of the therapeutic agent (e.g., growth factor), or pharmaceutically acceptable prodrug or salt thereof. In some embodiments, the otic formulation or composition comprises about 0.01% by weight of the therapeutic agent, or pharmaceutically acceptable prodrug or salt thereof. In some embodiments, the otic formulation or composition comprises about 0.02% by weight of the therapeutic agent, or pharmaceutically acceptable prodrug or salt thereof. In some embodiments, the otic formulation or composition comprises about 0.03% by weight of the therapeutic agent, or pharmaceutically acceptable prodrug or salt thereof. In some embodiments, the otic formulation or composition comprises about 0.04% by weight of the therapeutic agent, or pharmaceutically acceptable prodrug or salt thereof. In some embodiments, the otic formulation or composition comprises about 0.05% by weight of the therapeutic agent, or pharmaceutically acceptable prodrug or salt thereof. In some embodiments, the otic formulation or composition comprises about 0.06% by weight of the therapeutic agent, or pharmaceutically acceptable prodrug or salt thereof. In some embodiments, the otic formulation or composition comprises about 0.07% by weight of the therapeutic agent, or pharmaceutically acceptable prodrug or salt thereof. In some embodiments, the otic formulation or composition comprises about 0.08% by weight of the therapeutic agent, or pharmaceutically acceptable prodrug or salt thereof. In some embodiments, the otic formulation or composition comprises about 0.09% by weight of the therapeutic agent, or pharmaceutically acceptable prodrug or salt thereof. In some embodiments, the otic formulation or composition comprises about 0.1% by weight of the therapeutic agent, or pharmaceutically acceptable prodrug or salt thereof. In some embodiments, the otic formulation or composition comprises about 0.2% by weight of the therapeutic agent, or pharmaceutically acceptable prodrug or salt thereof. In some embodiments, the otic formulation or composition comprises about 0.3% by weight of the therapeutic agent, or pharmaceutically acceptable prodrug or salt thereof. In some embodiments, the otic formulation or composition comprises about 0.4% by weight of the therapeutic agent, or pharmaceutically acceptable prodrug or salt thereof. In some embodiments, the otic formulation or composition comprises about 0.5% by weight of the therapeutic agent, or pharmaceutically acceptable prodrug or salt thereof. In some embodiments, the otic formulation or composition comprises about 0.6% by weight of the therapeutic agent, or pharmaceutically acceptable prodrug or salt thereof. In some embodiments, the otic formulation or composition comprises about 0.7% by weight of the therapeutic agent, or pharmaceutically acceptable prodrug or salt thereof. In some embodiments, the otic formulation or composition comprises about 0.8% by weight of the therapeutic agent, or pharmaceutically acceptable prodrug or salt thereof. In some embodiments, the otic formulation or composition comprises about 0.9% by weight of the therapeutic agent, or pharmaceutically acceptable prodrug or salt thereof. In some embodiments, the otic formulation or composition comprises about 1% by weight of the therapeutic agent, or pharmaceutically acceptable prodrug or salt thereof. In some embodiments, the otic formulation or composition comprises about 2% by weight of the therapeutic agent, or pharmaceutically acceptable prodrug or salt thereof. In some embodiments, the otic formulation or composition comprises about 3% by weight of the therapeutic agent, or pharmaceutically acceptable prodrug or salt thereof. In some embodiments, the otic formulation or composition comprises about 4% by weight of the therapeutic agent, or pharmaceutically acceptable prodrug or salt thereof. In some embodiments, the otic formulation or composition comprises about 5% by weight of the therapeutic agent, or pharmaceutically acceptable prodrug or salt thereof. In some embodiments, the otic formulation or composition comprises about 6% by weight of the therapeutic agent, or pharmaceutically acceptable prodrug or salt thereof. In some embodiments, the otic formulation or composition comprises about 7% by weight of the therapeutic agent, or pharmaceutically acceptable prodrug or salt thereof. In some embodiments, the otic formulation or composition comprises about 8% by weight of the therapeutic agent, or pharmaceutically acceptable prodrug or salt thereof. In some embodiments, the otic formulation or composition comprises about 9% by weight of the therapeutic agent, or pharmaceutically acceptable prodrug or salt thereof. In some embodiments, the otic formulation or composition comprises about 10% by weight of the therapeutic agent, or pharmaceutically acceptable prodrug or salt thereof. In some embodiments, the otic formulation or composition comprises about 11% by weight of the therapeutic agent, or pharmaceutically acceptable prodrug or salt thereof. In some embodiments, the otic formulation or composition comprises about 12% by weight of the therapeutic agent, or pharmaceutically acceptable prodrug or salt thereof. In some embodiments, the otic formulation or composition comprises about 13% by weight of the therapeutic agent, or pharmaceutically acceptable prodrug or salt thereof. In some embodiments, the otic formulation or composition comprises about 14% by weight of the therapeutic agent, or pharmaceutically acceptable prodrug or salt thereof. In some embodiments, the otic formulation or composition comprises about 15% by weight of the therapeutic agent, or pharmaceutically acceptable prodrug or salt thereof. In some embodiments, the otic formulation or composition comprises about 16% by weight of the therapeutic agent, or pharmaceutically acceptable prodrug or salt thereof. In some embodiments, the otic formulation or composition comprises about 17% by weight of the therapeutic agent, or pharmaceutically acceptable prodrug or salt thereof. In some embodiments, the otic formulation or composition comprises about 18% by weight of the therapeutic agent, or pharmaceutically acceptable prodrug or salt thereof. In some embodiments, the otic formulation or composition comprises about 19% by weight of the therapeutic agent, or pharmaceutically acceptable prodrug or salt thereof. In some embodiments, the otic formulation or composition comprises about 20% by weight of the therapeutic agent, or pharmaceutically acceptable prodrug or salt thereof.

### Devices

Also contemplated herein are the use of devices for the delivery of the pharmaceutical formulations and compositions disclosed herein, or alternatively for the measurement or surveillance of the function of the auris formulations disclosed herein. For example, in one embodiment pumps, osmotic devices or other means of mechanically delivering pharmaceutical formulations and compositions are used for the delivery of the pharmaceutical formulations disclosed herein. Reservoir devices are optionally used with the pharmaceutical drug delivery units, and reside either internally along with the drug delivery unit, or externally of the auris structures.

Other embodiments contemplate the use of mechanical or imaging devices to monitor or survey the hearing, balance or other auris disorder. For example, magnetic resonance imaging (MRI) devices are specifically contemplated within the scope of the embodiments, wherein the MRI devices (for example, 3 Tesla MRI devices) are capable of evaluating Ménière Disease progression and subsequent treatment with the pharmaceutical formulations disclosed herein. See, Carfrae et al. Laryngoscope 118:501-505 (March 2008). Whole body scanners, or alternatively cranial scanners, are contemplated, as well as higher resolution (7 Tesla, 8 Tesla, 9.5 Tesla or 11 Tesla for humans) are optionally used in MRI scanning.

### Visualization of Otic Formulations

Also provided herein in some embodiments are otic formulations and compositions that comprise a dye (e.g., a Trypan blue dye, Evans blue dye) or other tracer compound. In some instances, addition of an auris-compatible dye to an otic formulation or composition described herein aids visualization of any administered formulation or composition in an ear (e.g., a rodent ear and/or a human ear). In certain embodiments, an otic formulation or composition comprising a dye or other tracer compound eliminates the need for invasive procedures that are currently used in animal models to monitor the concentrations of drugs in the endolymph and/or perilymph.

In some instances, intratympanic injections require the need of a specialist and the formulation or composition needs to be delivered to a specific site of the ear to maximize efficiency of the medication delivered. In certain instances, a visualization technique for any formulation or composition described herein allows for visualization of a dosing site (e.g., the round window) so that the medication is applied in the proper place. In some instances, a formulation or composition comprising a dye allows visualization of the formulation or composition during administration of the formulation to an ear (e.g., a human ear), ensures that the medication will be delivered at the intended site, and avoids any complications due to incorrect placement of a formulation or composition. The inclusion of a dye to help enhance the visualization of the formulation or composition when applied, and the ability to visually inspect the location of the formulation or composition after administration without further intervention, represents an advance over currently available methods for testing intratympanic therapeutics in animal models and/or human trials. In some embodiments, dyes that are compatible with the otic compositions described herein include Evans blue (e.g., 0.5% of the total weight of an otic formulation), Methylene blue (e.g., 1% of the total weight of an otic formulation), Isosulfan blue (e.g., 1% of the total weight of an otic formulation), Trypan blue (e.g., 0.15% of the total weight of an otic formulation), and/or indocyanine green (e.g., 25 mg/vial). Other common dyes, e.g., FD&C red 40, FD&C red 3, FD&C yellow 5, FD&C yellow 6, FD&C blue 1, FD&C blue2, FD&C green 3, fluorescence dyes (e.g., Fluorescein isothiocyanate, rhodamine, Alexa Fluors, DyLight Fluors) and/or dyes that are visualizable in conjunction with non-invasive imaging techniques such as MRI, CAT scans, PET scans or the like (e.g., Gadolinium-based MRI dyes, iodine-base dyes, barium-based dyes or the like) are also contemplated for use with any otic formulation or composition described herein. Other dyes that are compatible with any formulation or composition described herein are listed in the Sigma-Aldrich catalog under dyes (which is included herein by reference for such disclosure). In some embodiments, concentration of a dye in any otic formulation described herein is less than 2%, less than 1.5%, less than 1%, less than 0.5%, less than 0.25%, less than 0.1%, or less than 100 ppm of the total weight and/or volume of any formulation or composition described herein.

In certain embodiments of such auris-compatible formulations or compositions that comprise a dye, the ability to visualize a controlled release otic formulation or composition comprising a dye in an ear meets a long standing need for suitable testing methods that are applicable to the development of intratympanic otic formulations or compositions suitable for human use. In certain embodiments of such auris-compatible formulations or compositions that comprise a dye, the ability to visualize a controlled release otic formulation or composition comprising a dye allows for testing of any otic formulation described herein in human clinical trials.

### General Methods of Sterilization

The environment of the inner ear is an isolated environment. The endolymph and the perilymph are static fluids and are not in contiguous contact with the circulatory system. The blood—labyrinth— barrier (BLB), which includes a blood-endolymph barrier and a blood-perilymph barrier, consists of tight junctions between specialized epithelial cells in the labyrinth spaces (i.e., the vestibular and cochlear spaces). The presence of the BLB limits delivery of active agents to the isolated microenvironment of the inner ear. Auris hair cells are bathed in endolymphatic or perilymphatic fluids and cochlear recycling of potassium ions is important for hair cell function. When the inner ear is infected, there is an influx of leukocytes and/or immunoglobins (e.g. in response to a microbial infection) into the endolymph and/or the perilymph and the delicate ionic composition of inner ear fluids is upset by the influx of leukocytes and/or immunoglobins. In certain instances, a change in the ionic composition of inner ear fluids results in hearing loss, loss of balance and/or ossification of auditory structures. In certain instances, even trace amounts of pyrogens and/or microbes trigger infections and related physiological changes in the isolated microenvironment of the inner ear.

In one aspect, provided herein are otic formulations or compositions that are sterilized with stringent sterility requirements and are suitable for administration to the middle and/or inner ear. In some embodiments, the otic formulations or compositions described herein are auris compatible compositions. In some embodiment, the otic formulations or compositions are substantially free of pyrogens and/or microbes.

Provided herein are otic formulations or compositions that ameliorate or lessen otic disorders described herein. Further provided herein are methods comprising the administration of said otic formulations or compositions. In some embodiments, the formulations or compositions are sterilized. Included within the embodiments disclosed herein are means and processes for sterilization of a pharmaceutical composition disclosed herein for use in humans. The goal is to provide a safe pharmaceutical product, relatively free of infection causing micro-organisms. The U. S. Food and Drug Administration has provided regulatory guidance in the publication “Guidance for Industry: Sterile Drug Products Produced by Aseptic Processing” available at: http://www.fda.gov/cder/guidance/5882fnl.htm, which is incorporated herein by reference in its entirety. No specific guidelines are available for safe pharmaceutical products for treatment of the inner ear.

As used herein, sterilization means a process used to destroy or remove microorganisms that are present in a product or packaging. Any suitable method available for sterilization of objects and formulations or compositions is used. Available methods for the inactivation of microorganisms include, but are not limited to, the application of extreme heat, lethal chemicals, or gamma radiation. In some embodiments is a process for the preparation of an otic therapeutic formulation comprising subjecting the formulation to a sterilization method selected from heat sterilization, chemical sterilization, radiation sterilization or filtration sterilization. The method used depends largely upon the nature of the device or composition to be sterilized. Detailed descriptions of many methods of sterilization are given in Chapter 40 of Remington: The Science and Practice of Pharmacy published by Lippincott, Williams & Wilkins, and is incorporated by reference with respect to this subject matter.

### Sterilization by Heat

Many methods are available for sterilization by the application of extreme heat. One method is through the use of a saturated steam autoclave. In this method, saturated steam at a temperature of at least 121° C. is allowed to contact the object to be sterilized. The transfer of heat is either directly to the microorganism, in the case of an object to be sterilized, or indirectly to the microorganism by heating the bulk of an aqueous solution to be sterilized. This method is widely practiced as it allows flexibility, safety and economy in the sterilization process.

Dry heat sterilization is a method which is used to kill microorganisms and perform depyrogenation at elevated temperatures. This process takes place in an apparatus suitable for heating HEPA-filtered microorganism-free air to temperatures of at least 130-180° C. for the sterilization process and to temperatures of at least 230-250° C. for the depyrogenation process. Water to reconstitute concentrated or powdered formulations is also sterilized by autoclave.

### Chemical Sterilization

Chemical sterilization methods are an alternative for products that do not withstand the extremes of heat sterilization. In this method, a variety of gases and vapors with germicidal properties, such as ethylene oxide, chlorine dioxide, formaldehyde or ozone are used as the anti-apoptotic agents. The germicidal activity of ethylene oxide, for example, arises from its ability to serve as a reactive alkylating agent. Thus, the sterilization process requires the ethylene oxide vapors to make direct contact with the product to be sterilized.

### Radiation Sterilization

One advantage of radiation sterilization is the ability to sterilize many types of products without heat degradation or other damage. The radiation commonly employed is beta radiation or alternatively, gamma radiation from a ⁶⁰Co source. The penetrating ability of gamma radiation allows its use in the sterilization of many product types, including solutions, compositions and heterogeneous mixtures. The germicidal effects of irradiation arise from the interaction of gamma radiation with biological macromolecules. This interaction generates charged species and free radicals. Subsequent chemical reactions, such as rearrangements and cross-linking processes, result in the loss of normal function for these biological macromolecules. The formulations described herein are also optionally sterilized using beta irradiation.

### Filtration

Filtration sterilization is a method used to remove but not destroy microorganisms from solutions. Membrane filters are used to filter heat-sensitive solutions. Such filters are thin, strong, homogenous polymers of mixed cellulosic esters (MCE), polyvinylidene fluoride (PVF; also known as PVDF), or polytetrafluoroethylene (PTFE) and have pore sizes ranging from 0.1 to 0.22 μm. Solutions of various characteristics are optionally filtered using different filter membranes. For example, PVF and PTFE membranes are well suited to filtering organic solvents while aqueous solutions are filtered through PVF or MCE membranes. Filter apparatus are available for use on many scales ranging from the single point-of-use disposable filter attached to a syringe up to commercial scale filters for use in manufacturing plants. The membrane filters are sterilized by autoclave or chemical sterilization. Validation of membrane filtration systems is performed following standardized protocols (Microbiological Evaluation of Filters for Sterilizing Liquids, Vol 4, No. 3. Washington, D.C: Health Industry Manufacturers Association, 1981) and involve challenging the membrane filter with a known quantity (ca. 10⁷/cm²) of unusually small microorganisms, such as Brevundimonas diminuta (ATCC 19146).

Pharmaceutical formulations or compositions are optionally sterilized by passing through membrane filters. In some embodiments, formulations or compositions comprising nanoparticles (U.S. Pat. No. 6,139,870) or multilamellar vesicles (Richard et al., International Journal of Pharmaceutics (2006), 312(1-2):144-50) are amenable to sterilization by filtration through 0.22 μm filters without destroying their organized structure.

In some embodiments, the methods disclosed herein comprise sterilizing the formulation or compositions (or components thereof) by means of filtration sterilization. In another embodiment the otic formulation or composition comprises a particle wherein the particle formulation or composition is suitable for filtration sterilization. In a further embodiment said particle formulation or composition comprises particles of less than 300 nm in size, of less than 200 nm in size, of less than 100 nm in size. In another embodiment the otic formulation or composition comprises a particle formulation or composition wherein the sterility of the particle is ensured by sterile filtration of the precursor component solutions. In another embodiment the otic formulation or composition comprises a particle formulation or composition wherein the sterility of the particle formulation or composition is ensured by low temperature sterile filtration. In a further embodiment, said low temperature sterile filtration occurs at a temperature between 0 and 30° C., or between 0 and 20° C., or between 0 and 10° C., or between 10 and 20° C., or between 20 and 30° C. In another embodiment is a process for the preparation of an auris-acceptable particle formulation or composition comprising: filtering the aqueous solution containing the particle formulation or composition at low temperature through a sterilization filter; lyophilizing the sterile solution; and reconstituting the particle formulation or composition with sterile water prior to administration. In some embodiments, a formulation described herein is manufactured as a suspension in a single vial formulation containing the micronized active pharmaceutical ingredient. A single vial formulation is prepared by aseptically mixing a sterile poloxamer solution with sterile micronized active ingredient (e.g., ketamine) and transferring the formulation to sterile pharmaceutical containers. In some embodiments, a single vial containing a formulation described herein as a suspension is resuspended before dispensing and/or administration.

In specific embodiments, filtration and/or filling procedures are carried out at about 5° C. below the gel temperature (Tgel) of a formulation described herein and with viscosity below a theoretical value of 100 cP to allow for filtration in a reasonable time using a peristaltic pump.

In another embodiment the otic formulation or composition comprises a nanoparticle formulation or composition wherein the nanoparticle formulation or composition is suitable for filtration sterilization. In a further embodiment the nanoparticle formulation or composition comprises nanoparticles of less than 300 nm in size, of less than 200 nm in size, or of less than 100 nm in size. In another embodiment the otic formulation or composition comprises a microsphere formulation or composition wherein the sterility of the microsphere is ensured by sterile filtration of the precursor organic solution and aqueous solutions. In another embodiment, the sterility of the otic formulation or composition is ensured by low temperature sterile filtration. In a further embodiment, the low temperature sterile filtration occurs at a temperature between 0 and 30° C., or between 0 and 20° C., or between 0 and 10° C., or between 10 and 20° C., or between 20 and 30° C. In another embodiment is a process for the preparation of an auris-acceptable thermoreversible gel formulation comprising: filtering the aqueous solution containing the thermoreversible gel components at low temperature through a sterilization filter; lyophilizing the sterile solution; and reconstituting the thermoreversible gel formulation with sterile water prior to administration.

In certain embodiments, the active ingredients are dissolved in a suitable vehicle (e.g. a buffer) and sterilized separately (e.g. by heat treatment, filtration, gamma radiation); the remaining excipients are sterilized in a separate step by a suitable method (e.g. filtration and/or irradiation of a cooled mixture of excipients); the two solutions that were separately sterilized are then mixed aseptically to provide a final otic formulation or composition.

In some instances, conventionally used methods of sterilization (e.g., heat treatment (e.g., in an autoclave), gamma irradiation, filtration) lead to irreversible degradation of the therapeutic agent in the formulation or composition.

In some instances, conventionally used methods of sterilization (e.g., heat treatment (e.g., in an autoclave), gamma irradiation, filtration) lead to irreversible degradation of polymeric components (e.g., thermosetting, gelling or mucoadhesive polymer components) and/or the active agent in the formulation. In some instances, sterilization of an auris formulation by filtration through membranes (e.g., 0.2 □M membranes) is not possible if the formulation comprises thixotropic polymers that gel during the process of filtration.

Accordingly, provided herein are methods for sterilization of auris formulations that prevent degradation of polymeric components (e.g., thermosetting and/or gelling and/or mucoadhesive polymer components) and/or the therapeutic agent during the process of sterilization. In some embodiments, degradation of the therapeutic agent is reduced or eliminated through the use of specific pH ranges for buffer components and specific proportions of gelling agents in the formulations. In some embodiments, the choice of an appropriate gelling agent and/or thermosetting polymer allows for sterilization of formulations described herein by filtration. In some embodiments, the use of an appropriate thermosetting polymer and an appropriate copolymer (e.g., a gelling agent) in combination with a specific pH range for the formulation allows for high temperature sterilization of formulations described with substantially no degradation of the therapeutic agent or the polymeric excipients. An advantage of the methods of sterilization provided herein is that, in certain instances, the formulations are subjected to terminal sterilization via autoclaving without any loss of the active agent and/or excipients and/or polymeric components during the sterilization step and are rendered substantially free of microbes and/or pyrogens.

### Microorganisms

Provided herein are otic formulations or compositions that ameliorate or lessen otic disorders described herein. Further provided herein are methods comprising the administration of said otic formulations or compositions. In some embodiments, the formulations or compositions are substantially free of microorganisms. Acceptable sterility levels are based on applicable standards that define therapeutically acceptable otic formulations or compositions, including but not limited to United States Pharmacopeia Chapters <1111> et seq. For example, acceptable sterility levels include 10 colony forming units (cfu) per gram of formulation or composition, 50 cfu per gram of formulation or composition, 100 cfu per gram of formulation or composition, 500 cfu per gram of formulation or composition or 1000 cfu per gram of formulation or composition. In addition, acceptable sterility levels include the exclusion of specified objectionable microbiological agents. By way of example, specified objectionable microbiological agents include but are not limited to Escherichia coli (E. coli), Salmonella sp., Pseudomonas aeruginosa (P. aeruginosa) and/or other specific microbial agents.

Sterility of the otic formulation is confirmed through a sterility assurance program in accordance with United States Pharmacopeia Chapters <61>, <62> and <71>. A key component of the sterility assurance quality control, quality assurance and validation process is the method of sterility testing. Sterility testing, by way of example only, is performed by two methods. The first is direct inoculation wherein a sample of the formulation to be tested is added to growth medium and incubated for a period of time up to 21 days. Turbidity of the growth medium indicates contamination. Drawbacks to this method include the small sampling size of bulk materials which reduces sensitivity, and detection of microorganism growth based on a visual observation. An alternative method is membrane filtration sterility testing. In this method, a volume of product is passed through a small membrane filter paper. The filter paper is then placed into media to promote the growth of microorganisms. This method has the advantage of greater sensitivity as the bulk product is sampled. The commercially available Millipore Steritest sterility testing system is optionally used for determinations by membrane filtration sterility testing. For the filtration testing of creams or ointments Steritest filter system No. TLHVSL210 are used. For the filtration testing of emulsions or viscous products Steritest filter system No. TLAREM210 or TDAREM210 are used. For the filtration testing of pre-filled syringes Steritest filter system No. TTHASY210 are used. For the filtration testing of material dispensed as an aerosol or foam Steritest filter system No. TTHVA210 are used. For the filtration testing of soluble powders in ampoules or vials Steritest filter system No. TTHADA210 or TTHADV210 are used.

Testing for E. coli and Salmonella includes the use of lactose broths incubated at 30-35° C. for 24-72 hours, incubation in MacConkey and/or EMB agars for 18-24 hours, and/or the use of Rappaport medium. Testing for the detection of P. aeruginosa includes the use of NAC agar. United States Pharmacopeia Chapter <62> further enumerates testing procedures for specified objectionable microorganisms.

In certain embodiments, any otic formulation or composition described herein has less than about 60 colony forming units (CFU), less than about 50 colony forming units, less than about 40 colony forming units, or less than about 30 colony forming units of microbial agents per gram of formulation. In certain embodiments, the otic formulations or compositions described herein are formulated to be isotonic with the endolymph and/or the perilymph.

### Endotoxins

Provided herein are otic formulations or compositions that ameliorate or lessen otic disorders described herein. Further provided herein are methods comprising the administration of said otic formulations or compositions. In some embodiments, the otic formulations or compositions are substantially free of endotoxins. An additional aspect of the sterilization process is the removal of by-products from the killing of microorganisms (hereinafter, “Product”). The process of depyrogenation removes pyrogens from the sample. Pyrogens are endotoxins or exotoxins which induce an immune response. An example of an endotoxin is the lipopolysaccharide (LPS) molecule found in the cell wall of gram-negative bacteria. While sterilization procedures such as autoclaving or treatment with ethylene oxide kill the bacteria, the LPS residue induces a proinflammatory immune response, such as septic shock. Because the molecular size of endotoxins varies widely, the presence of endotoxins is expressed in “endotoxin units” (EU). One EU is equivalent to 100 picograms of E. coli LPS. In some cases, humans develop a response to as little as 5 EU/kg of body weight. The sterility is expressed in any units as recognized in the art. In certain embodiments, otic formulations or compositions described herein contain lower endotoxin levels (e.g. <4 EU/kg of body weight of a subject) when compared to conventionally acceptable endotoxin levels (e.g., 5 EU/kg of body weight of a subject). In some embodiments, the otic formulation or composition has less than about 5 EU/kg of body weight of a subject. In other embodiments, the otic formulation or composition has less than about 4 EU/kg of body weight of a subject. In additional embodiments, the otic formulation or composition has less than about 3 EU/kg of body weight of a subject. In additional embodiments, the otic formulation or composition has less than about 2 EU/kg of body weight of a subject.

In some embodiments, the otic formulation or composition has less than about 5 EU/kg of formulation. In other embodiments, the otic therapeutic formulation or composition has less than about 4 EU/kg of formulation. In additional embodiments, the otic formulation or composition has less than about 3 EU/kg of formulation. In some embodiments, the otic formulation or composition has less than about 5 EU/kg Product. In other embodiments, the otic formulation or composition has less than about 1 EU/kg Product. In additional embodiments, the otic formulation or composition has less than about 0.2 EU/kg Product. In some embodiments, the otic formulation or composition has less than about 5 EU/g of unit or Product. In other embodiments, the otic formulation or composition has less than about 4 EU/g of unit or Product. In additional embodiments, the otic formulation or composition has less than about 3 EU/g of unit or Product. In some embodiments, the otic formulation or composition has less than about 5 EU/mg of unit or Product. In other embodiments, the otic formulation or composition has less than about 4 EU/mg of unit or Product. In additional embodiments, the otic formulation or composition has less than about 3 EU/mg of unit or Product. In certain embodiments, otic formulations or compositions described herein contain from about 1 to about 5 EU/mL of formulation or composition. In certain embodiments, otic formulations or compositions described herein contain from about 2 to about 5 EU/mL of formulation or composition, from about 3 to about 5 EU/mL of formulation or composition, or from about 4 to about 5 EU/mL of formulation or composition.

In certain embodiments, otic formulations or compositions described herein contain lower endotoxin levels (e.g. <0.5 EU/mL of formulation or composition) when compared to conventionally acceptable endotoxin levels (e.g., 0.5 EU/mL of formulation or composition). In some embodiments, the otic formulation or composition has less than about 0.5 EU/mL of formulation or composition. In other embodiments, the otic formulation or composition has less than about 0.4 EU/mL of formulation or composition. In additional embodiments, the otic formulation or composition has less than about 0.2 EU/mL of formulation or composition.

Pyrogen detection, by way of example only, is performed by several methods. Suitable tests for sterility include tests described in United States Pharmacopoeia (USP) <71> Sterility Tests (23rd edition, 1995). The rabbit pyrogen test and the Limulus amebocyte lysate test are both specified in the United States Pharmacopeia Chapters <85> and <151> (USP23/NF 18, Biological Tests, The United States Pharmacopeial Convention, Rockville, Md., 1995). Alternative pyrogen assays have been developed based upon the monocyte activation-cytokine assay. Uniform cell lines suitable for quality control applications have been developed and have demonstrated the ability to detect pyrogenicity in samples that have passed the rabbit pyrogen test and the Limulus amebocyte lysate test (Taktak et al, J. Pharm. Pharmacol. (1990), 43:578-82). In an additional embodiment, the otic formulation or composition is subject to depyrogenation. In a further embodiment, the process for the manufacture of the otic formulation or composition comprises testing the formulation for pyrogenicity. In certain embodiments, the formulations or compositions described herein are substantially free of pyrogens.

### pH and Osmolarity

Described herein are otic formulations or compositions with an ionic balance that is compatible with the perilymph and/or the endolymph and does not cause any change in cochlear potential. In specific embodiments, osmolarity/osmolality of the present formulations or compositions is adjusted, for example, by the use of appropriate salt concentrations (e.g., concentration of sodium salts) or the use of tonicity agents which renders the formulations or compositions endolymph-compatible and/or perilymph compatible (i.e. isotonic with the endolymph and/or perilymph). In some instances, the endolymph-compatible and/or perilymph-compatible formulations or compositions described herein cause minimal disturbance to the environment of the inner ear and cause minimum discomfort (e.g., vertigo) to a mammal (e.g., a human) upon administration. In some embodiments, the formulations or compositions described herein are free of preservatives and cause minimal disturbance (e.g., change in pH or osmolarity, irritation) in auditory structures. In some embodiments, the formulations or compositions described herein comprise antioxidants that are non-irritating and/or non-toxic to otic structures.

As used herein, “practical osmolarity” means the osmolarity of a formulation that is measured by including the active agent and all excipients except the gelling and/or the thickening agent (e.g., polyoxyethylene-polyoxypropylene copolymers, carboxymethylcellulose or the like). The practical osmolarity of a formulation described herein is measured by any suitable method, e.g., a freezing point depression method as described in Viegas et. al., Int. J. Pharm., 1998, 160, 157-162. In some instances, the practical osmolarity of a formulation described herein is measured by vapor pressure osmometry (e.g., vapor pressure depression method) that allows for determination of the osmolarity of a formulation at higher temperatures. In some instances, vapor pressure depression method allows for determination of the osmolarity of a formulation comprising a gelling agent (e.g., a thermoreversible polymer) at a higher temperature wherein the gelling agent is in the form of a gel. In some embodiments, the practical osmolality of an otic formulation described herein is from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, the formulations described herein have a practical osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L.

In some embodiments, the osmolarity at a target site of action (e.g., the perilymph) is about the same as the delivered osmolarity (i.e., osmolarity of materials that cross or penetrate the round window membrane) of any formulation described herein. In some embodiments, the formulations described herein have a deliverable osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L.

The main cation present in the endolymph is potassium. In addition the endolymph has a high concentration of positively charged amino acids. The main cation present in the perilymph is sodium. In certain instances, the ionic composition of the endolymph and perilymph regulate the electrochemical impulses of hair cells. In certain instances, any change in the ionic balance of the endolymph or perilymph results in a loss of hearing due to changes in the conduction of electrochemical impulses along otic hair cells. In some embodiments, a composition or formulation disclosed herein does not disrupt the ionic balance of the perilymph. In some embodiments, a composition or formulation disclosed herein has an ionic balance that is the same as or substantially the same as the perilymph. In some embodiments, a composition or formulation disclosed herein does not disrupt the ionic balance of the endolymph. In some embodiments, a composition or formulation disclosed herein has an ionic balance that is the same as or substantially the same as the endolymph. In some embodiments, a composition or formulation described herein is formulated to provide an ionic balance that is compatible with inner ear fluids (i.e., endolymph and/or perilymph).

The endolymph and the perilymph have a pH that is close to the physiological pH of blood. The endolymph has a pH range of about 7.2-7.9; the perilymph has a pH range of about 7.2-7.4. The in situ pH of the proximal endolymph is about 7.4 while the pH of distal endolymph is about 7.9.

In some embodiments, the pH of a formulation or composition described herein is adjusted (e.g., by use of a buffer) to an endolymph-compatible pH range of about 7.0 to 8.0, and a preferred pH range of about 7.2-7.9. In some embodiments, the pH of the formulations or compositions described herein is adjusted (e.g., by use of a buffer) to a perilymph—compatible pH of about 7.0-7.6, and a preferred pH range of about 7.2-7.4.

In some embodiments, useful formulations or compositions also include one or more pH adjusting agents or buffering agents. Suitable pH adjusting agents or buffers include, but are not limited to acetate, bicarbonate, ammonium chloride, citrate, phosphate, pharmaceutically acceptable salts thereof and combinations or mixtures thereof.

In some embodiments, the pharmaceutical formulations or compositions described herein are stable with respect to pH over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 1 month, at least about 2 months, at least about 3 months, at least about 4 months, at least about 5 months, or at least about 6 months. In other embodiments, the formulations or compositions described herein are stable with respect to pH over a period of at least about 1 week. Also described herein are formulations or compositions that are stable with respect to pH over a period of at least about 1 month.

### Tonicity Agents

In general, the endolymph has a higher osmolality than the perilymph. For example, the endolymph has an osmolality of about 304 mOsm/kg H₂O while the perilymph has an osmolality of about 294 mOsm/kg H₂O. In some embodiments, formulations or compositions described herein are formulated to provide an osmolarity of about 250 to about 320 mM (osmolality of about 250 to about 320 mOsm/kg H₂O); and preferably about 270 to about 320 mM (osmolality of about 270 to about 320 mOsm/kg H₂O). In certain embodiments, tonicity agents are added to the formulations described herein in an amount as to provide a practical osmolality of an otic formulation of about 100 mOsm/kg to about 1000 mOsm/kg, from about 200 mOsm/kg to about 800 mOsm/kg, from about 250 mOsm/kg to about 500 mOsm/kg, from about 250 mOsm/kg to about 350 mOsm/kg, or from about 280 mOsm/kg to about 320 mOsm/kg. In some embodiments, the formulations described herein have a practical osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 280 mOsm/L to about 320 mOsm/L, or about 250 mOsm/L to about 320 mOsm/L.

In specific embodiments, osmolarity/osmolality of the present formulations or compositions is adjusted, for example, by the use of appropriate salt concentrations (e.g., concentration of potassium salts) or the use of tonicity agents which renders the formulations or compositions endolymph-compatible and/or perilymph-compatible (i.e. isotonic with the endolymph and/or perilymph. In some instances, the endolymph-compatible and/or perilymph-compatible formulations or compositions described herein cause minimal disturbance to the environment of the inner ear and cause minimum discomfort (e.g., vertigo and/or nausea) to a mammal upon administration.

In some embodiments, the deliverable osmolarity of any formulation described herein is designed to be isotonic with the targeted otic structure (e.g., endolymph, perilymph, or the like). In specific embodiments, auris formulations described herein are formulated to provide a delivered perilymph-suitable osmolarity at the target site of action of about 250 to about 320 mOsm/L and preferably about 270 to about 320 mOsm/L. In specific embodiments, auris formulations described herein are formulated to provide a delivered perilymph-suitable osmolality at the target site of action of about 250 to about 320 mOsm/kg H₂O or an osmolality of about 270 to about 320 mOsm/kg H₂O. In specific embodiments, the deliverable osmolarity/osmolality of the formulations (i.e., the osmolarity/osmolality of the formulation in the absence of gelling or thickening agents (e.g., thermoreversible gel polymers) is adjusted, for example, by the use of appropriate salt concentrations (e.g., concentration of potassium or sodium salts) or the use of tonicity agents which renders the formulations endolymph-compatible and/or perilymph-compatible (i.e. isotonic with the endolymph and/or perilymph) upon delivery at the target site. The osmolarity of a formulation comprising a thermoreversible gel polymer is an unreliable measure due to the association of varying amounts of water with the monomeric units of the polymer. The practical osmolarity of a formulation (i.e., osmolarity in the absence of a gelling or thickening agent (e.g. a thermoreversible gel polymer) is a reliable measure and is measured by any suitable method (e.g., freezing point depression method, vapor depression method). In some instances, the formulations described herein provide a deliverable osmolarity (e.g., at a target site (e.g., perilymph) that causes minimal disturbance to the environment of the inner ear and causes minimum discomfort (e.g., vertigo and/or nausea) to a mammal upon administration.

In some embodiments, any formulation or composition described herein is isotonic with the perilymph. Isotonic formulations or compositions are provided by the addition of a tonicity agent. Suitable tonicity agents include, but are not limited to any pharmaceutically acceptable sugar, salt or any combinations or mixtures thereof, such as, but not limited to dextrose, glycerin, mannitol, sorbitol, sodium chloride, and other electrolytes.

Useful otic formulations or compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite, and ammonium sulfate.

In further embodiments, the tonicity agents are present in an amount as to provide a final osmolality of an otic formulation or composition of about 100 mOsm/kg to about 500 mOsm/kg, from about 200 mOsm/kg to about 400 mOsm/kg, from about 250 mOsm/kg to about 350 mOsm/kg or from about 280 mOsm/kg to about 320 mOsm/kg. In some embodiments, the formulations or compositions described herein have a osmolarity of about 100 mOsm/L to about 500 mOsm/L, about 200 mOsm/L to about 400 mOsm/L, about 250 mOsm/L to about 350 mOsm/L, or about 280 mOsm/L to about 320 mOsm/L. In some embodiments, the osmolarity of any formulation or composition described herein is designed to be isotonic with the targeted otic structure (e.g., endolymph, perilymph or the like). In some embodiments, the formulations described herein have a pH and/or practical osmolarity as described herein, and have a concentration of active pharmaceutical ingredient from about 0.0001% to about 60%, from about 0.001% to about 40%, from about 0.01% to about 20%, from about 0.01% to about 10%, from about 0.01% to about 7.5%, from about 0.01% to about 6%, from about 0.01 to about 5%, from about 0.1 to about 10%, or from about 0.1 to about 6% of the active ingredient by weight of the formulation.

In some embodiments, the formulations or compositions described herein have a pH and osmolarity as described herein, and have a concentration of active pharmaceutical ingredient between about 1 μM and about 10 μM, between about 1 mM and about 100 mM, between about 0.1 mM and about 100 mM, between about 0.1 mM and about 100 nM. In some embodiments, the formulations or compositions described herein have a pH and osmolarity as described herein, and have a concentration of active pharmaceutical ingredient between about 0.01-about 20%, between about 0.01-about 10%, between about 0.01-about 7%, between about 0.01-5%, between about 0.01-about 3%, between about 0.01-about 2% of the active ingredient by weight of the formulation or composition. In some embodiments, the formulations or composition described herein have a pH and osmolarity as described herein, and have a concentration of active pharmaceutical ingredient between about 0.1-about 70 mg/mL, between about 1 mg-about 70 mg/mL, between about 1 mg-about 50 mg/mL, between about 1 mg/mL and about 20 mg/mL, between about 1 mg/mL to about 10 mg/mL, between about 1 mg/mL to about 5 mg/mL, or between about 0.5 mg/mL to about 5 mg/mL of the active agent by volume of the formulation or composition.

### Particle Size

Size reduction is used to increase surface area and/or modulate formulation dissolution properties. It is also used to maintain a consistent average particle size distribution (PSD) (e.g., micrometer-sized particles, nanometer-sized particles or the like) for any formulation or composition described herein. In some embodiments, the formulation or composition comprises micrometer-sized particles. In some embodiments, the formulation or composition comprises nanometer-sized particles. In some instances, any formulation or composition described herein comprises multiparticulates, i.e., a plurality of particle sizes (e.g., micronized particles, nano-sized particles, non-sized particles); i.e., the formulation or composition is a multiparticulate formulation or composition. In some embodiments, any formulation or composition described herein comprises one or more multiparticulate (e.g., micronized) therapeutic agents. Micronization is a process of reducing the average diameter of particles of a solid material. Micronized particles are from about micrometer-sized in diameter to about picometer—sized in diameter. In some embodiments, the use of multiparticulates (e.g., micronized particles) of a therapeutic agent, or an otic agent, allows for extended and/or sustained release of the therapeutic agent from any formulation described herein compared to a formulation or composition comprising non-multiparticulate (e.g., non-micronized) therapeutic agent. In some instances, formulations or compositions containing multiparticulate (e.g., micronized) therapeutic agents are ejected from a 1 mL syringe adapted with a 27G needle without any plugging or clogging. In some embodiments, the therapeutic agent is essentially in the form of micronized particles. In some embodiments, the therapeutic agent is essentially in the form of microsized particles. In some embodiments, the therapeutic agent is essentially in the form of nanosized particles.

In some embodiments, the particle size of the formulation or composition described herein increases the retention time of the formulation or composition described herein. In some embodiments, the particle size of the formulation or composition described herein provides slow release of the therapeutic agent. In some embodiments, the particle size of the formulation or composition described herein provides sustained release of the therapeutic agent. In some embodiments, the particle size is less than 450 nm, less than 400 nm, less than 350 nm, less than 300 nm, less than 275 nm, less than 250 nm, less than 225 nm, less than 200 nm in size, less than 175 nm, less than 150 nm, or less than 125 nm, or less than 100 nm. In some embodiments, the particle size is less than 300 nm. In some embodiments, the particle size is less than 250 nm. In some embodiments, the particle size is less than 200 nm.

In some instances, any particle in any formulation or composition described herein is a coated particle (e.g., a coated micronized particle) and/or a microsphere and/or a liposomal particle. Particle size reduction techniques include, by way of example, grinding, milling (e.g., air-attrition milling (jet milling), ball milling), coacervation, high pressure homogenization, spray drying and/or supercritical fluid crystallization. In some instances, particles are sized by mechanical impact (e.g., by hammer mills, ball mill and/or pin mills). In some instances, particles are sized via fluid energy (e.g., by spiral jet mills, loop jet mills, and/or fluidized bed jet mills). In some embodiments formulations described herein comprise crystalline particles. In some embodiments, formulations or compositions described herein comprise amorphous particles. In some embodiments, formulations or compositions described herein comprise therapeutic agent particles wherein the therapeutic agent is a free base, or a salt, or a prodrug of a therapeutic agent, or any combination thereof.

In some specific embodiments, any otic formulation or composition described herein comprises one or more micronized therapeutic agents. In some of such embodiments, a micronized therapeutic agent comprises micronized particles, coated (e.g., with an extended release coat) micronized particles, or a combination thereof. In some of such embodiments, a micronized therapeutic agent comprising micronized particles, coated micronized particles, or a combination thereof, comprises a therapeutic agent as a free base, a salt, a prodrug or any combination thereof.

### Auris-Acceptable Gel Formulations/Compositions

In some embodiments, the auris-acceptable formulations or compositions described herein are gel formulations or gel compositions.

In some embodiments, the otic gel formulations or compositions that include at least therapeutic agent and a pharmaceutically acceptable diluent(s), excipient(s), or carrier(s). In some embodiments, the otic gel formulations or compositions include other medicinal or pharmaceutical agents; carriers; adjuvants; preserving, stabilizing, wetting or emulsifying agents; solution promoters; salts for regulating the osmotic pressure; and/or buffers. In some embodiments, the otic gel formulations or compositions comprises (i) a therapeutic agent, (ii) a gelling and viscosity enhancing agent, (iii) a pH adjusting agent, and (iv) sterile water.

Gels, sometimes referred to as jellies, have been defined in various ways. For example, the United States Pharmacopoeia defines gels as semisolid systems consisting of either suspensions made up of small inorganic particles or large organic molecules interpenetrated by a liquid. Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums (e.g., tragacanth). In some embodiments, single-phase gels are generally aqueous but will also be made using alcohols and oils. Two-phase gels consist of a network of small discrete particles.

Gels can also be classified as being hydrophobic or hydrophilic. In certain embodiments, the base of a hydrophobic gel consists of a liquid paraffin with polyethylene or fatty oils gelled with colloidal silica or aluminum or zinc soaps. In contrast, the base of hydrophilic gels usually consists of water, glycerol, or propylene glycol gelled with a suitable gelling agent (e.g., tragacanth, starch, cellulose derivatives, carboxyvinylpolymers, and magnesium-aluminum silicates). In certain embodiments, the rheology of the formulations or devices disclosed herein is pseudo plastic, plastic, thixotropic, or dilatant.

In one embodiment the enhanced viscosity auris-acceptable formulation described herein is not a liquid at room temperature. In certain embodiments, the enhanced viscosity formulation is characterized by a phase transition between room temperature and body temperature (including an individual with a serious fever, e.g., up to about 42° C.). In some embodiments, the phase transition occurs at about 1° C. below body temperature, at about 2° C. below body temperature, at about 3° C. below body temperature, at about 4° C. below body temperature, at about 6° C. below body temperature, at about 8° C. below body temperature, or at about 10° C. below body temperature. In some embodiments, the phase transition occurs at about 15° C. below body temperature, at about 20° C. below body temperature, or at about 25° C. below body temperature. In specific embodiments, the gelation temperature (Tgel) of a formulation described herein is about 20° C., about 25° C., or about 30° C. In certain embodiments, the gelation temperature (Tgel) of a formulation described herein is about 35° C. or about 40° C. In one embodiment, administration of any formulation described herein at about body temperature reduces or inhibits vertigo associated with intratympanic administration of otic formulations. Included within the definition of body temperature is the body temperature of a healthy individual or an unhealthy individual, including an individual with a fever (up to ˜42° C.). In some embodiments, the pharmaceutical formulations or devices described herein are liquids at about room temperature and are administered at or about room temperature, reducing or ameliorating side effects such as, for example, vertigo.

Polymers composed of polyoxypropylene and polyoxyethylene form thermoreversible gels when incorporated into aqueous solutions. These polymers have the ability to change from the liquid state to the gel state at temperatures close to body temperature, therefore allowing useful formulations that are applied to the targeted auris structure(s). The liquid state-to-gel state phase transition is dependent on the polymer concentration and the ingredients in the solution.

Poloxamer 407 (PF-127) is a nonionic surfactant composed of polyoxyethylene-polyoxypropylene copolymers. Other poloxamers include 188 (F-68 grade), 237 (F-87 grade), and 338 (F-108 grade). Aqueous solutions of poloxamers are stable in the presence of acids, alkalis, and metal ions. PF-127 is a commercially available polyoxyethylene-polyoxypropylene triblock copolymer of general formula E106 P70 E106, with an average molar mass of 13,000. The polymer can be further purified by suitable methods that will enhance gelation properties of the polymer. It contains approximately 70% ethylene oxide, which accounts for its hydrophilicity. It is one of the series of poloxamer ABA block copolymers, whose members share the chemical formula shown below.

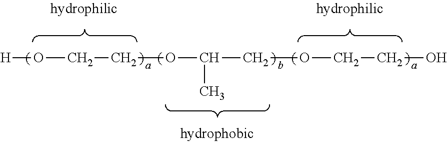

PF-127 is of particular interest since concentrated solutions (>20% w/w) of the copolymer are transformed from low viscosity transparent solutions to solid gels on heating to body temperature. This phenomenon, therefore, suggests that when placed in contact with the body, the gel preparation will form a semi-solid structure and a sustained release depot. Furthermore, PF-127 has good solubilizing capacity, low toxicity and is, therefore, considered a good medium for drug delivery systems.

In some embodiments, the amount of thermoreversible polymer in any formulation described herein is about 0.01%, about 0.05%, about 0.1%, about 0.5%, about 1%, about 5%, about 10%, about 15%, about 20%, about 25%, about 30%, about 35%, or about 40% of the total weight of the formulation. In some embodiments, the amount of thermoreversible polymer in any formulation described herein is about 0.01%, about 0.05%, about 0.1%, about 0.5%, about 1%, about 5%, about 10%, about 11%, about 12%, about 13%, about 14%, about 15%, about 16%, about 17%, about 18%, about 19%, about 20%, about 21%, about 22%, about 23%, about 24%, or about 25% of the total weight of the formulation. In some embodiments, the amount of thermoreversible polymer (e.g., pluronic F127) in any formulation described herein is about 7.5% of the total weight of the formulation. In some embodiments, the amount of thermoreversible polymer (e.g., pluronic F127) in any formulation described herein is about 10% of the total weight of the formulation. In some embodiments, the amount of thermoreversible polymer (e.g., pluronic F127) in any formulation described herein is about 11% of the total weight of the formulation. In some embodiments, the amount of thermoreversible polymer (e.g., pluronic F127) in any formulation described herein is about 12% of the total weight of the formulation. In some embodiments, the amount of thermoreversible polymer (e.g., pluronic F127) in any formulation described herein is about 13% of the total weight of the formulation. In some embodiments, the amount of thermoreversible polymer (e.g., pluronic F127) in any formulation described herein is about 14% of the total weight of the formulation. In some embodiments, the amount of thermoreversible polymer (e.g., pluronic F127) in any formulation described herein is about 15% of the total weight of the formulation. In some embodiments, the amount of thermoreversible polymer (e.g., pluronic F127) in any formulation described herein is about 16% of the total weight of the formulation. In some embodiments, the amount of thermoreversible polymer (e.g., pluronic F127) in any formulation described herein is about 17% of the total weight of the formulation. In some embodiments, the amount of thermoreversible polymer (e.g., pluronic F127) in any formulation described herein is about 18% of the total weight of the formulation. In some embodiments, the amount of thermoreversible polymer (e.g., pluronic F127) in any formulation described herein is about 19% of the total weight of the formulation. In some embodiments, the amount of thermoreversible polymer (e.g., pluronic F127) in any formulation described herein is about 20% of the total weight of the formulation. In some embodiments, the amount of thermoreversible polymer (e.g., pluronic F127) in any formulation described herein is about 21% of the total weight of the formulation. In some embodiments, the amount of thermoreversible polymer (e.g., pluronic F127) in any formulation described herein is about 23% of the total weight of the formulation. In some embodiments, the amount of thermoreversible polymer (e.g., pluronic F127) in any formulation described herein is about 25% of the total weight of the formulation.

In an alternative embodiment, the thermogel is a PEG-PLGA-PEG triblock copolymer (Jeong et al, Nature (1997), 388:860-2; Jeong et al, J. Control. Release (2000), 63:155-63; Jeong et al, Adv. Drug Delivery Rev. (2002), 54:37-51). The polymer exhibits sol-gel behavior over a concentration of about 5% w/w to about 40% w/w. Depending on the properties desired, the lactide/glycolide molar ratio in the PLGA copolymer ranges from about 1:1 to about 20:1. The resulting copolymers are soluble in water and form a free-flowing liquid at room temperature but form a hydrogel at body temperature. A commercially available PEG-PLGA-PEG triblock copolymer is RESOMER RGP t50106 manufactured by Boehringer Ingelheim. This material is composed of a PGLA copolymer of 50:50 poly(DL-lactide-co-glycolide), is 10% w/w of PEG, and has a molecular weight of about 6000.

ReGel® is a tradename of MacroMed Incorporated for a class of low molecular weight, biodegradable block copolymers having reverse thermal gelation properties as described in U.S. Pat. Nos. 6,004,573, 6,117,949, 6,201,072, and 6,287,588. It also includes biodegradable polymeric drug carriers disclosed in pending U.S. patent application Ser. Nos. 09/906,041, 09/559,799 and 10/919,603. The biodegradable drug carrier comprises ABA-type or BAB-type triblock copolymers, or mixtures thereof, wherein the A-blocks are relatively hydrophobic and comprise biodegradable polyesters or poly(orthoester)s, and the B-blocks are relatively hydrophilic and comprise polyethylene glycol (PEG), said copolymers having a hydrophobic content of between 50.1 to 83% by weight and a hydrophilic content of between 17 to 49.9% by weight, and an overall block copolymer molecular weight of between 2000 and 8000 Daltons. The drug carriers exhibit water solubility at temperatures below normal mammalian body temperatures and undergo reversible thermal gelation to then exist as a gel at temperatures equal to physiological mammalian body temperatures. The biodegradable, hydrophobic A polymer block comprises a polyester or poly(ortho ester), in which the polyester is synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxyhexanoic acid, γ-butyrolactone, γ-hydroxybutyric acid, δ-valerolactone, δ-hydroxyvaleric acid, hydroxybutyric acids, malic acid, and copolymers thereof and having an average molecular weight of between about 600 and 3000 Daltons. The hydrophilic B-block segment is preferably polyethylene glycol (PEG) having an average molecular weight of between about 500 and 2200 Daltons.

Additional biodegradable thermoplastic polyesters include AtriGel (provided by Atrix Laboratories, Inc.) and/or those disclosed, e.g., in U.S. Pat. Nos. 5,324,519; 4,938,763; 5,702,716; 5,744,153; and 5,990,194; wherein the suitable biodegradable thermoplastic polyester is disclosed as a thermoplastic polymer. Examples of suitable biodegradable thermoplastic polyesters include polylactides, polyglycolides, polycaprolactones, copolymers thereof, terpolymers thereof, and any combinations thereof. In some such embodiments, the suitable biodegradable thermoplastic polyester is a polylactide, a polyglycolide, a copolymer thereof, a terpolymer thereof, or any combination thereof. In one embodiment, the biodegradable thermoplastic polyester is 50/50 poly(DL-lactide-co-glycolide) having a carboxy terminal group; is present in about 30 wt. % to about 40 wt. % of the formulation; and has an average molecular weight of about 23,000 to about 45,000. Alternatively, in another embodiment, the biodegradable thermoplastic polyester is 75/25 poly (DL-lactide-co-glycolide) without a carboxy terminal group; is present in about 40 wt. % to about 50 wt. % of the formulation; and has an average molecular weight of about 15,000 to about 24,000. In further or alternative embodiments, the terminal groups of the poly(DL-lactide-co-glycolide) are either hydroxyl, carboxyl, or ester depending upon the method of polymerization. Polycondensation of lactic or glycolic acid provides a polymer with terminal hydroxyl and carboxyl groups. Ring-opening polymerization of the cyclic lactide or glycolide monomers with water, lactic acid, or glycolic acid provides polymers with the same terminal groups. However, ring-opening of the cyclic monomers with a monofunctional alcohol such as methanol, ethanol, or 1-dodecanol provides a polymer with one hydroxyl group and one ester terminal groups. Ring-opening polymerization of the cyclic monomers with a diol such as 1,6-hexanediol or polyethylene glycol provides a polymer with only hydroxyl terminal groups.

Since the polymer systems of thermoreversible gels dissolve more completely at reduced temperatures, methods of solubilization include adding the required amount of polymer to the amount of water to be used at reduced temperatures. Generally after wetting the polymer by shaking, the mixture is capped and placed in a cold chamber or in a thermostatic container at about 0-10° C. in order to dissolve the polymer. The mixture is stirred or shaken to bring about a more rapid dissolution of the thermoreversible gel polymer. The active agent and various additives such as buffers, salts, and preservatives are subsequently added and dissolved. In some instances the active agent and/or other pharmaceutically active agent is suspended if it is insoluble in water. The pH is modulated by the addition of appropriate buffering agents. Round window membrane mucoadhesive characteristics are optionally imparted to a thermoreversible gel by incorporation of round window membrane mucoadhesive carbomers, such as Carbopol® 934P, to the formulation (Majithiya et al., AAPS PharmSciTech (2006), 7(3), p. E1; EP0551626, both of which is incorporated herein by reference for such disclosure).

In one embodiment are auris-acceptable pharmaceutical gel formulations which do not require the use of an added viscosity enhancing agent or viscosity modulating agent. Such gel formulations incorporate at least one pharmaceutically acceptable buffer. In one aspect is a gel formulation and a pharmaceutically acceptable buffer. In another embodiment, the pharmaceutically acceptable excipient or carrier is a gelling agent.

In one specific embodiment of the auris-acceptable controlled-release formulations described herein, the active agent is provided in a gel matrix, also referred to herein as “auris-acceptable gel formulations”, “auris interna-acceptable gel formulations”, “auris media-acceptable gel formulations”, “auris externa-acceptable gel formulations”, “auris gel formulations”, or variations thereof. All of the components of the gel formulation must be compatible with the targeted auris structure. Further, the gel formulations provide controlled-release of the active agent to the desired site within the targeted auris structure; in some embodiments, the gel formulation also has an immediate or rapid release component for delivery of the active agent to the desired target site. In other embodiments, the gel formulation has a sustained release component for delivery of the active agent. In some embodiments, the auris gel formulations are biodegradable. In other embodiments, the auris gel formulations include a mucoadhesive excipient to allow adhesion to the external mucous layer of the round window membrane. In yet other embodiments, the auris gel formulations include a penetration enhancer excipient; in further embodiments, the auris gel formulation contains a viscosity enhancing agent sufficient to provide a viscosity of from about 10 to about 1,000,000 centipoise, from about 500 and 1,000,000 centipoise; from about 750 to about 1,000,000 centipoise; from about 1000 to about 1,000,000 centipoise; from about 1000 to about 400,000 centipoise; from about 2000 to about 100,000 centipoise; from about 3000 to about 50,000 centipoise; from about 4000 to about 25,000 centipoise; from about 5000 to about 20,000 centipoise; or from about 6000 to about 15,000 centipoise. In some embodiments, the auris gel formulation contains a viscosity enhancing agent sufficient to provide a viscosity of from about 50,0000 to about 1,000,000 centipoise. In some embodiments, the auris gel formulation contains a viscosity enhancing agent sufficient to provide a viscosity of from about 50,0000 to about 3,000,000 centipoise.

In other embodiments, the otic pharmaceutical formulations described herein further provide an auris-acceptable hydrogel; in yet other embodiments, the otic pharmaceutical formulations provide an auris-acceptable microsphere or microparticle; in still other embodiments, the otic pharmaceutical formulations provide an auris-acceptable liposome. In some embodiments, the otic pharmaceutical formulations provide an auris-acceptable foam; in yet other embodiments, the otic pharmaceutical formulations provide an auris-acceptable paint; in still further embodiments, otic pharmaceutical formulations provide an auris-acceptable in situ forming spongy material. In some embodiments, the otic pharmaceutical formulations provide an auris-acceptable solvent release gel. In some embodiments, the otic pharmaceutical formulations provide an actinic radiation curable gel. Further embodiments include a thermoreversible gel in the otic pharmaceutical formulation, such that upon preparation of the gel at room temperature or below, the formulation is a fluid, but upon application of the gel into or near the auris interna and/or auris media target site, including the tympanic cavity, round window membrane, or the crista fenestrae cochleae, the otic-pharmaceutical formulation stiffens or hardens into a gel-like substance.

In further or alternative embodiments, the otic gel formulations are capable of being administered on or near the round window membrane via intratympanic injection. In other embodiments, the otic gel formulations are administered on or near the round window or the crista fenestrae cochleae through entry via a post-auricular incision and surgical manipulation into or near the round window or the crista fenestrae cochleae area. Alternatively, the otic gel formulation is applied via syringe and needle, wherein the needle is inserted through the tympanic membrane and guided to the area of the round window or crista fenestrae cochleae. The otic gel formulations are then deposited on or near the round window or crista fenestrae cochleae for localized treatment of autoimmune otic disorders. In other embodiments, the otic gel formulations are applied via microcathethers implanted into the patient, and in yet further embodiments the formulations are administered via a pump device onto or near the round window membrane. In still further embodiments, the otic gel formulations are applied at or near the round window membrane via a microinjection device. In yet other embodiments, the otic gel formulations are applied in the tympanic cavity. In some embodiments, the otic gel formulations are applied on the tympanic membrane. In still other embodiments, the otic gel formulations are applied onto or in the auditory canal.

### Preservatives

In some embodiments, the otic formulations or compositions described herein is free of preservatives. In some embodiments, a formulation or composition disclosed herein comprises a preservative. Suitable auris-acceptable preservatives for use in a formulation or composition disclosed herein include, but are not limited to benzoic acid, boric acid, p-hydroxybenzoates, benzyl alcohol, lower alkyl alcohols (e.g., ethanol, butanol or the like), quaternary compounds, stabilized chlorine dioxide, mercurials, such as merfen and thimerosal, mixtures of the foregoing and the like. Suitable preservatives for use with a formulation disclosed herein are not ototoxic. In some embodiments, a formulation or composition disclosed herein does not include a preservative that is ototoxic. In some embodiments, a formulation or composition disclosed herein does not include benzalkonium chloride or benzethonium chloride.

In certain embodiments, any otic formulation or composition described herein has an endotoxin level of less than 0.5 EU/kg, less than 0.4 EU/kg or less than 0.3 EU/kg. In certain embodiments, any otic formulation or composition described herein has less than about 60 colony forming units (CFU), has less than about 50 colony forming units, has less than about 40 colony forming units, has less than about 30 colony forming units of microbial agents per gram of formulation or composition. In certain embodiments, any controlled release formulation or composition described herein is substantially free of pyrogens.

In a further embodiment, the preservative is, by way of example only, an antimicrobial agent, within the formulation or composition presented herein. In one embodiment, the formulation or composition includes a preservative such as by way of example only, methyl paraben. In another embodiment, the methyl paraben is at a concentration of about 0.05% to about 1.0%, about 0.1% to about 0.2%. In certain embodiments, the preservative employed in any auris-compatible formulation described herein is an antioxidant (e.g., butyl hydroxytoluene (BHT) or the like, as described herein). In certain embodiments, an antioxidant preservative is non-toxic and/or non-irritating to the inner ear environment.

### Retention Time

In some embodiments, the formulation or composition has a retention time in the ear of about 5 minutes, about 15 minutes, about 30 minutes, about 1 hour, about 4 hours, about 6 hours, about 12 hours, about 18 hours, about 1 day, about 2 days, about 3 days, about 4 days, about 5 days, about 6 days, about 7 days, about 10 days, about 12 days, about 14 days, about 18 days, about 21 days, about 25 days, about 30 days, about 45 days, about 2 months, about 3 months, about 4 months, about 5 months, about 6 months, about 9 months or about 1 year. In some embodiments, the formulation or composition has a retention time in the ear of at least 5 minutes, at least 15 minutes, at least 30 minutes, at least 1 hour, at least 4 hours, at least 6 hours, at least 12 hours, at least 18 hours, at least 1 day, at least 2 days, at least 3 days, at least 4 days, at least 5 days, at least 6 days, at least 7 days, at least 10 days, at least 12 days, at least 14 days, at least 18 days, at least 21 days, at least 25 days, at least 30 days, at least 45 days, at least 2 months, at least 3 months, at least 4 months, at least 5 months, at least 6 months, at least 9 months or at least 1 year.

In some embodiments, the ear is the outer ear, middle ear, or inner ear. In some embodiments, the ear is the middle ear. In some embodiments, the ear is the inner ear. In some embodiments, the ear is the outer ear. In some embodiments, the outer ear is the external auditory canal, the outer surface of the tympanic membrane, or a combination thereof.

### Modes of Otic Administration

In some embodiments, the auris formulations or compositions described herein are administered into the ear canal, or in the vestibule of the ear. Access to, for example, the vestibular and cochlear apparatus occurs through the auris media including the round window membrane, the oval window/stapes footplate, the annular ligament and through the otic capsule/temporal bone. In some embodiments, otic administration of the formulations or compositions described herein avoids toxicity associated with systemic administration (e.g., hepatotoxicity, cardiotoxicity, gastrointestinal side effects, and renal toxicity) of the active agents. In some instances, localized administration in the ear allows an active agent to reach a target organ (e.g., inner ear) in the absence of systemic accumulation of the active agent. In some instances, local administration to the ear provides a higher therapeutic index for an active agent that otherwise have dose-limiting systemic toxicity.

Provided herein are modes of treatment for otic formulations or compositions that ameliorate or lessen otic disorders described herein. Drugs delivered to the inner ear have been administered systemically via oral, intravenous or intramuscular routes. However, systemic administration for pathologies local to the inner ear increases the likelihood of systemic toxicities and adverse side effects and creates a non-productive distribution of drug in which high levels of drug are found in the serum and correspondingly lower levels are found at the inner ear.

Provided herein are methods comprising the administration of said auris formulations or compositions on or near the round window membrane via intratympanic injection. In some embodiments, a composition disclosed herein is administered on or near the round window or the crista fenestrae cochleae through entry via a post-auricular incision and surgical manipulation into or near the round window or the crista fenestrae cochleae area. Alternatively, a formulation or composition disclosed herein is applied via syringe and needle, wherein the needle is inserted through the tympanic membrane and guided to the area of the round window or crista fenestrae cochleae. In some embodiments, a formulation or composition disclosed herein is then deposited on or near the round window or crista fenestrae cochleae for localized treatment. In other embodiments, a formulation or composition disclosed herein is applied via microcathethers implanted into the patient, and in yet further embodiments a composition disclosed herein is administered via a pump device onto or near the round window membrane. In still further embodiments, a formulation or composition disclosed herein is applied at or near the round window membrane via a microinjection device. In yet other embodiments, a formulation or composition disclosed herein is applied in the tympanic cavity. In some embodiments, a formulation or composition disclosed herein is applied on the tympanic membrane. In still other embodiments, a formulation or composition disclosed herein is applied onto or in the auditory canal. The formulations or compositions described herein, and modes of administration thereof, are also applicable to methods of direct instillation or perfusion of the inner ear compartments. Thus, the formulations or compositions described herein are useful in surgical procedures including, by way of non-limiting examples, cochleostomy, labyrinthotomy, mastoidectomy, stapedectomy, endolymphatic sacculotomy or the like.

### Intratympanic Injections

In some embodiments, a surgical microscope is used to visualize the tympanic membrane. In some embodiments, the tympanic membrane is anesthetized by any suitable method (e.g., use of phenol, lidocaine, and xylocaine). In some embodiments, the anterior-superior and posterior-inferior quadrants of the tympanic membrane are anesthetized.

In some embodiments, a puncture is made in the tympanic membrane to vent any gases behind the tympanic membrane. In some embodiments, a puncture is made in the anterior-superior quadrant of the tympanic membrane to vent any gases behind the tympanic membrane. In some embodiments, the puncture is made with a needle (e.g., a 25 gauge needle). In some embodiments, the puncture is made with a laser (e.g., a CO₂ laser). In one embodiment the delivery system is a syringe and needle apparatus that is capable of piercing the tympanic membrane and directly accessing the round window membrane or crista fenestrae cochleae of the auris interna.

In one embodiment, the needle is a hypodermic needle used for instant delivery of the formulation. The hypodermic needle is a single use needle or a disposable needle. In some embodiments, a syringe is used for delivery of the pharmaceutically acceptable otic agent-containing compositions as disclosed herein wherein the syringe has a press-fit (Luer) or twist-on (Luer-lock) fitting. In one embodiment, the syringe is a hypodermic syringe. In another embodiment, the syringe is made of plastic or glass. In yet another embodiment, the hypodermic syringe is a single use syringe. In a further embodiment, the glass syringe is capable of being sterilized. In yet a further embodiment, the sterilization occurs through an autoclave. In another embodiment, the syringe comprises a cylindrical syringe body wherein the formulation is stored before use. In other embodiments, the syringe comprises a cylindrical syringe body wherein the pharmaceutically acceptable otic formulations or compositions as disclosed herein is stored before use which conveniently allows for mixing with a suitable pharmaceutically acceptable buffer. In other embodiments, the syringe contains other excipients, stabilizers, suspending agents, diluents, or a combination thereof to stabilize or otherwise stably store the otic agent or other pharmaceutical compounds contained therein.

In some embodiments, the syringe comprises a cylindrical syringe body wherein the body is compartmentalized in that each compartment is able to store at least one component of the auris-acceptable otic formulation. In a further embodiment, the syringe having a compartmentalized body allows for mixing of the components prior to injection into the auris media or auris interna. In other embodiments, the delivery system comprises multiple syringes, each syringe of the multiple syringes contains at least one component of the formulation such that each component is pre-mixed prior to injection or is mixed subsequent to injection. In a further embodiment, the syringes disclosed herein comprise at least one reservoir wherein the at least one reservoir comprises an otic agent, or a pharmaceutically acceptable buffer, or a viscosity enhancing agent, or a combination thereof. Commercially available injection devices are optionally employed in their simplest form as ready-to-use plastic syringes with a syringe barrel, needle assembly with a needle, plunger with a plunger rod, and holding flange, to perform an intratympanic injection.

In some embodiments, a needle is used to deliver the formulations or compositions described herein. In some embodiments, a needle punctures the posterior-inferior quadrant of the tympanic membrane. In some embodiments, the needle is a standard gauge needle. In some embodiments, the needle is a narrow gauge needle. In some embodiments, the needle is wider than an 18 gauge needle. In another embodiment, the needle gauge is from about 18 gauge to about 30 gauge. In some embodiments, the needle gauge is from about 20 gauge to about 30 gauge. In some embodiments, the needle gauge is from about 25 gauge to about 30 gauge. In some embodiments, the needle gauge is about 18 gauge, about 19 gauge, about 20 gauge, about 21 gauge, about 22 gauge, about 23 gauge, about 24 gauge, about 25 gauge, about 26 gauge, about 27 gauge, about 28 gauge, about 29 gauge, or about 30 gauge. In a further embodiment, the needle is a 25 gauge needle. Depending upon the thickness or viscosity of a formulation or composition disclosed herein, the gauge level of the syringe or hypodermic needle is varied accordingly. In some embodiments, the formulations or compositions described herein are liquids and are administered via narrow gauge needles or cannulas (e.g., 22 gauge needle, 25 gauge needle, or cannula), minimizing damage to the tympanic membrane upon administration. The formulations or compositions described herein are administered with minimal discomfort to a patient.

In some embodiments, an otoendoscope (e.g., about 1.7 mm in diameter) is used to visualize the round window membrane. In some embodiments, any obstructions to the round window membrane (e.g., a false round window membrane, a fat plug, fibrous tissue) are removed.

In some embodiments, a formulation or composition disclosed herein is injected onto the round window membrane. In some embodiments, 0.1 to 0.5 cc of a formulation or composition disclosed herein is injected onto the round window membrane.

In some embodiments, the tympanic membrane puncture is left to heal spontaneously. In some embodiments, a paper patch myringoplasty is performed by a trained physician. In some embodiments, a tympanoplasty is performed by a trained physician. In some embodiments, an individual is advised to avoid water. In some embodiments, a cotton ball soaked in petroleum-jelly is utilized as a barrier to water and other environmental agents.

### Other Delivery Routes

In some embodiments, a formulation or composition disclosed herein is administered locally to the outer ear, such as the external auditory canal, the outer surface of the tympanic membrane, or a combination thereof. In some embodiments, the formulations or compositions described herein are not administered through the tympanic membrane.

In some embodiments, a formulation or composition disclosed herein is administered to the inner ear. In some embodiments, a formulation or composition disclosed herein is administered to the inner ear via an incision in the stapes footplate. In some embodiments, a formulation or composition disclosed herein is administered to the cochlea via a cochleostomy. In some embodiments, a formulation or composition disclosed herein is administered to the vestibular apparatus (e.g., semicircular canals or vestibule).

In some embodiments, a formulation or composition disclosed herein is applied via syringe and needle. In other embodiments, a formulation or composition disclosed herein is applied via microcatheters implanted into the patient. In some embodiments, a formulation or composition disclosed herein is administered via a pump device. In still further embodiments, a formulation or composition disclosed herein is applied via a microinjection device. In some embodiments, a formulation or composition disclosed herein is administered via a prosthesis, a cochlear implant, a constant infusion pump, or a wick.

In some embodiments, the delivery device is an apparatus designed for administration of therapeutic agents to the middle and/or inner ear. By way of example only: GYRUS Medical GmbH offers micro-otoscopes for visualization of and drug delivery to the round window niche; Arenberg has described a medical treatment device to deliver fluids to inner ear structures in U.S. Pat. Nos. 5,421,818; 5,474,529; and 5,476,446, each of which is incorporated by reference herein for such disclosure. U.S. patent application Ser. No. 08/874,208, which is incorporated herein by reference for such disclosure, describes a surgical method for implanting a fluid transfer conduit to deliver therapeutic agents to the inner ear. U.S. Patent Application Publication 2007/0167918, which is incorporated herein by reference for such disclosure, further describes a combined otic aspirator and medication dispenser for intratympanic fluid sampling and medicament application.

### Dosage

In some embodiments, auris formulations or compositions described herein are controlled release formulations, and are administered at reduced dosing frequency compared to the current standard of care. In certain instances, when an auris formulation or composition is administered via intratympanic injection, a reduced frequency of administration alleviates discomfort caused by multiple intratympanic injections in individuals undergoing treatment for a middle and/or inner ear disease, disorder or condition. In certain instances, a reduced frequency of administration of intratympanic injections reduces the risk of permanent damage (e.g., perforation) to the ear drum. In some embodiments, formulations or compositions described herein provide a constant, sustained, extended, delayed or pulsatile rate of release of an active agent into the inner ear environment and thus avoid any variability in drug exposure in treatment of otic disorders.

The formulations or compositions containing the compound(s) described herein are administered for prophylactic and/or therapeutic treatments. In therapeutic applications, the formulations or compositions are administered to a patient already suffering from a disease, condition or disorder, in an amount sufficient to cure or at least partially arrest the symptoms of the disease, disorder or condition. Amounts effective for this use will depend on the severity and course of the disease, disorder or condition, previous therapy, the patient's health status and response to the drugs, and the judgment of the treating physician.

The amount of a given agent that will correspond to such an amount will vary depending upon factors such as the particular compound, disease condition and its severity, but is nevertheless routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-50 mg per administration, preferably 1-15 mg per administration. In some embodiments, the desired dose is conveniently presented in a single dose or as divided doses administered simultaneously (or over a short period of time) or at appropriate intervals.

### Frequency of Administration

In the case wherein the patient's condition does not improve, upon the doctor's discretion the administration of the compounds is administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds are given continuously; alternatively, the dose of drug being administered are temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday are from 10%400%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. Patients, however, require intermittent treatment on a long-term basis upon any recurrence of symptoms in some embodiments.

In some embodiments, the initial administration is of a particular formulation and the subsequent administration is of a different formulation or active pharmaceutical ingredient.

### Otic Surgery and Implants

In some embodiments, the pharmaceutical formulations or compositions described herein are used in combination with (e.g., implantation, short-term use, long-term use, or removal of) implants (e.g., cochlear implants). As used herein, implants include auris-interna or auris-media medical devices, examples of which include cochlear implants, hearing sparing devices, hearing-improvement devices, short electrodes, micro-prostheses or piston-like prostheses; needles; stem cell transplants; drug delivery devices; any cell-based therapeutic; or the like. In some instances, the implants are used in conjunction with a patient experiencing hearing loss. In some instances, the hearing loss is present at birth. In some instances, the hearing loss is associated with conditions such as AIED, bacterial meningitis or the like that lead to osteoneogenesis and/or nerve damage with rapid obliteration of cochlear structures and profound hearing loss.

In some instances, an implant is an immune cell or a stem cell transplant in the ear. In some instances, an implant is a small electronic device that has an external portion placed behind the ear, and a second portion that is surgically placed under the skin that helps provide a sense of sound to a person who is profoundly deaf or severely hard-of-hearing. By way of example, such cochlear medical device implants bypass damaged portions of the ear and directly stimulate the auditory nerve. In some instances cochlear implants are used in single sided deafness. In some instances cochlear implants are used for deafness in both ears.

In some embodiments, administration of a formulation or composition or device described herein in combination with an otic intervention (e.g., an intratympanic injection, a stapedectomy, a medical device implant, or a cell-based transplant) delays or prevents collateral damage to auris structures, e.g., irritation, cell damage, cell death, osteoneogenesis, and/or further neuronal degeneration, caused by the external otic intervention (e.g., installation of an external device and/or cells in the ear). In some embodiments, administration of a formulation or device described herein in combination with an implant allows for a more effective restoration of hearing loss compared to an implant alone.

In some embodiments, administration of formulation or composition or device described herein reduces damage to cochlear structures caused by underlying conditions (e.g., bacterial meningitis, autoimmune ear disease (AIED)) allowing for successful cochlear device implantation. In some embodiments, administration of a formulation or composition or device described herein, in conjunction with otic surgery, medical device implantation, and/or cell transplantation, reduces or prevents cell damage and/or death (e.g., auris sensory hair cell death and/or damage) associated with otic surgery, medical device implantation, and/or cell transplantation.

In some embodiments, administration of a formulation or composition or device described herein in conjunction with a cochlear implant or stem cell transplant has a trophic effect (e.g., promotes healthy growth of cells and/or healing of tissue in the area of an implant or transplant). In some embodiments, a trophic effect is desirable during otic surgery or during intratympanic injection procedures. In some embodiments, a trophic effect is desirable after installation of a medical device or after a cell transplant. In some of such embodiments, the formulations or compositions or devices described herein are administered via direct cochlear injection, through a cochleostomy or via deposition on the round window.

In some embodiments, administration of the formulations or compositions described herein reduces inflammation and/or infections associated with otic surgery, implantation of a medical device or a cell transplant. In some instances, perfusion of a surgical area with a formulation described herein reduces or eliminates post-surgical and/or post-implantation complications (e.g., inflammation, hair cell damage, neuronal degeneration, osteoneogenesis, or the like). In some instances, perfusion of a surgical area with a formulation or composition described herein reduces post-surgery or post-implantation recuperation time. In some embodiments, a medical device is coated with a formulation or composition described herein prior to implantation in the ear.

In one aspect, the formulations or compositions described herein, and modes of administration thereof, are applicable to methods of direct perfusion of the inner ear compartments. Thus, the formulations or compositions described herein are useful in combination with otic interventions. In some embodiments, an otic intervention is an implantation procedure (e.g., implantation of a hearing device in the cochlea). In some embodiments, an otic intervention is a surgical procedure including, by way of non-limiting examples, cochleostomy, labyrinthotomy, mastoidectomy, stapedectomy, stapedotomy, endolymphatic sacculotomy, tympanostomy, or the like. In some embodiments, the inner ear compartments are perfused with a formulation or composition described herein prior to otic intervention, during otic intervention, or after otic intervention, or a combination thereof.

In some embodiments, when perfusion is carried out in combination with otic intervention, the formulations or compositions are immediate release compositions. In some of such embodiments, the immediate release formulations described herein are substantially free of extended release components.

### Kits and Other Articles of Manufacture

The disclosure also provides kits for preventing, treating or ameliorating the symptoms of a diseases or disorder in a mammal. Such kits generally will comprise one or more of the pharmaceutically acceptable compositions as disclosed herein, and instructions for using the kit. The disclosure also contemplates the use of one or more of the formulations or compositions, in the manufacture of medicaments for treating, abating, reducing, or ameliorating the symptoms of a disease, dysfunction, or disorder in a mammal, such as a human that has, is suspected of having, or at risk for developing an auris interna disorder.

In some embodiments, a kit disclosed herein comprises a needle that penetrates a tympanic membrane and/or a round window. In some embodiments, a kit disclosed herein further comprises a hydrogel with a penetration enhancer (e.g., an alkylglycoside and/or a saccharide alkyl ester).

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

The articles of manufacture provided herein contain packaging materials. Packaging materials for use in packaging pharmaceutical products presented herein. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations or compositions of the compounds and formulations or compositions provided herein are contemplated as are a variety of treatments for any disease, disorder, or condition that would benefit by extended release administration of a therapeutic agent to the auris interna.

In some embodiments, a kit will typically include one or more additional containers, each with one or more of various materials (such as reagents, optionally in concentrated form, and/or devices) desirable from a commercial and user standpoint for use of a formulation or composition described herein. Non-limiting examples of such materials include, but not limited to, buffers, diluents, filters, needles, syringes; carrier, package, container, vial, and/or tube labels listing contents and/or instructions for use, and package inserts with instructions for use. A set of instructions will also typically be included.

In a further embodiment, a label is on or associated with the container. In yet a further embodiment, a label is on a container when letters, numbers or other characters forming the label are attached, molded or etched into the container itself; a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.

In certain embodiments, the pharmaceutical formulations or compositions are presented in a pack or dispenser device which contains one or more unit dosage forms containing a compound provided herein. In another embodiment, the pack for example contains metal or plastic foil, such as a blister pack. In a further embodiment, the pack or dispenser device is accompanied by instructions for administration. In yet a further embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. In another embodiment, such notice, for example, is the labeling approved by the U.S. Food and Drug Administration for prescription drugs, or the approved product insert. In yet another embodiment, compositions containing a compound provided herein formulated in a compatible pharmaceutical carrier are also prepared, placed in an appropriate container, and labeled for treatment of an indicated condition.

### NON-LIMITING EMBODIMENTS

Embodiment 1: A method of treating an otic disease or condition associated with a synapses-affecting event in a subject in need thereof, the method comprising administering an otic formulation comprising a therapeutically effective amount of a growth factor and an auris-acceptable vehicle within 14 days after onset of the synapses-damaging event.

Embodiment 2: The method of embodiment 1, wherein the growth factor is brain-derived neurotrophic factor (BDNF), ciliary neurotrophic factor (CNTF), glial cell-line derived neurotrophic factor (GDNF), neurotrophin-3, neurotrophin-4, or any combination thereof.

Embodiment 3: The method of embodiment 1 or embodiment 2, wherein the growth factor is brain-derived neurotrophic factor (BDNF).

Embodiment 4: The method of any one of embodiments 1-3, wherein the auris-acceptable vehicle is an auris-acceptable gel.

Embodiment 5: The method of embodiment 4, wherein the auris-acceptable gel is a thermoreversible gel.

Embodiment 6: The method of embodiment 4 or embodiment 5, wherein the auris-acceptable gel has a gelation viscosity from about 15,000 cP and about 3,000,000 cP.

Embodiment 7: The method of embodiment 6, wherein the auris-acceptable gel has a gelation viscosity from about 100,000 cP to about 500,000 cP.

Embodiment 8: The method of embodiment 6, wherein the auris-acceptable gel has a gelation viscosity from about 250,000 cP to about 500,000 cP.

Embodiment 9: The method of any one of embodiments 4-8, wherein the auris-acceptable gel is capable of being injected by a narrow gauge needle or cannula through the tympanic membrane.

Embodiment 10: The method of any one of embodiments 4-9, wherein the otic formulation has an osmolarity from about 100 mOsm/L to about 1000 mOsm/L.

Embodiment 11: The method of embodiment 10, wherein the otic formulation has an osmolarity from about 150 to about 500 mOsm/L.

Embodiment 12: The method of embodiment 10, wherein the otic formulation has an osmolarity from about 200 to about 400 mOsm/L.

Embodiment 13: The method of embodiment 10, wherein the otic formulation has an osmolarity from about 250 to about 320 mOsm/L.

Embodiment 14: The method of any one of embodiments 4-13, wherein the otic formulation has a gelation temperature from about 19° C. to about 42° C.

Embodiment 15: The method of any one of embodiments 4-14, wherein the otic formulation has a pH from about 7.0 to about 8.0.

Embodiment 16: The method of any one of embodiments 4-15, wherein the auris-acceptable gel comprises a copolymer of polyoxyethylene and polyoxypropylene.

Embodiment 17: The method of embodiment 16, wherein the copolymer of polyoxyethylene and polyoxypropylene is poloxamer 407.

Embodiment 18: The method of embodiment 17, wherein the otic formulation comprises from about 14 wt % to about 18 wt % poloxamer 407.

Embodiment 19: The method of embodiment 17, wherein the otic formulation comprises from about 15 wt % to about 17 wt % poloxamer 407.

Embodiment 20: The method of embodiment 17, wherein the otic formulation comprises about 16 wt % poloxamer 407.

Embodiment 21: The method of any one of embodiments 1-3, wherein the auris-acceptable vehicle comprises triglycerides comprising medium chain fatty acids.

Embodiment 22: The method of embodiment 21, wherein the triglycerides are derived from glycerol and medium chain fatty acids.

Embodiment 23: The method of embodiment 21 or embodiment 22, wherein each medium chain fatty acid independently comprises 6 to 12 carbon atoms in the carbon chain.

Embodiment 24: The method of embodiment 21 or embodiment 22, wherein each medium chain fatty acid independently comprises 8 to 12 carbon atoms in the carbon chain.

Embodiment 25: The method of any one of embodiments 21-24, wherein the medium chain fatty acids are saturated medium chain fatty acids, unsaturated medium chain fatty acids, or any combinations thereof.

Embodiment 26: The method of any one of embodiments 21-24, wherein the medium chain fatty acids are caproic acid (hexanoic acid), enanthic acid (heptanoic acid), caprylic acid (octanoic acid), pelargonic acid (nonanoic acid), capric acid (decanoic acid), undecylenic acid (undec-10-enoic acid), lauric acid (dodecanoic acid), or any combinations thereof.

Embodiment 27: The method of embodiment 21, wherein the triglycerides comprising medium chain fatty acids are balassee oil, coconut oil, cohune oil, palm kernel oil, tucum oil, or any combinations thereof.

Embodiment 28: The method of any one of embodiments 21-27, wherein the otic formulation comprises at least about 50% by weight of the triglycerides.

Embodiment 29: The method of any one of embodiments 21-27, wherein the otic formulation comprises from about 50% to about 99.99% by weight of the triglycerides, about 55% to about 99.99% by weight of the triglycerides, about 60% to about 99.99% by weight of the triglycerides, about 65% to about 99.99% by weight of the triglycerides, about 70% to about 99.99% by weight of the triglycerides, about 75% to about 99.99% by weight of the triglycerides, about 80% to about 99.99% by weight of the triglycerides, about 85% to about 99.99% by weight of the triglycerides, about 90% to about 99.99% by weight of the triglycerides, or about 95% to about 99.99% by weight of the triglycerides.

Embodiment 30: The method of embodiment 28 or embodiment 29, wherein the otic formulation has triglycerides in an amount that is sufficient to allow delivery of the formulation via a narrow gauge needle.

Embodiment 31: The method of any one of embodiments 21-30, wherein the otic formulation further comprises at least one viscosity modulating agent.

Embodiment 32: The method of embodiment 31, wherein the at least one viscosity modulating agent is silicon dioxide, povidone, carbomer, poloxamer, or a combination thereof.

Embodiment 33: The method of embodiment 32, wherein the viscosity modulating agent is silicon dioxide.

Embodiment 34: The method of embodiment 32, wherein the viscosity modulating agents are silicon dioxide and povidone.

Embodiment 35: The method of embodiment 34, wherein the otic formulation comprises between about 0.01% to about 20% by weight of the povidone, about 0.01% to about 15% by weight of the povidone, about 0.01% to about 10% by weight of the povidone, about 0.01% to about 7% by weight of the povidone, about 0.01% to about 5% by weight of the povidone, about 0.01% to about 3% by weight of the povidone, about 0.01% to about 2% by weight of the povidone, or about 0.01% to about 1% by weight of the povidone.

Embodiment 36: The method of embodiment 32, wherein the viscosity modulating agents are silicon dioxide and carbomer.

Embodiment 37: The method of embodiment 36, wherein the otic formulation comprises between about 0.01% to about 20% by weight of the carbomer, about 0.01% to about 15% by weight of the carbomer, about 0.01% to about 10% by weight of the carbomer, about 0.01% to about 7% by weight of the carbomer, about 0.01% to about 5% by weight of the carbomer, about 0.01% to about 3% by weight of the carbomer, about 0.01% to about 2% by weight of the carbomer, or about 0.01% to about 1% by weight of the carbomer.

Embodiment 38: The method of embodiment 32, wherein the viscosity modulating agents are silicon dioxide and poloxamer.

Embodiment 39: The method of embodiment 38, wherein the otic formulation comprises between about 0.01% to about 20% by weight of the poloxamer, about 0.01% to about 15% by weight of the poloxamer, about 0.01% to about 10% by weight of the poloxamer, about 0.01% to about 7% by weight of the poloxamer, about 0.01% to about 5% by weight of the poloxamer, about 0.01% to about 3% by weight of the poloxamer, about 0.01% to about 2% by weight of the poloxamer, or about 0.01% to about 1% by weight of the poloxamer.

Embodiment 40: The method of any one of embodiments 32-39, wherein the otic formulation comprises between about 0.01% to about 10% by weight of the silicon dioxide, about 0.01% to about 7% by weight of the silicon dioxide, about 0.01% to about 5% by weight of the silicon dioxide, about 0.01% to about 3% by weight of the silicon dioxide, about 0.01% to about 2% by weight of the silicon dioxide, or about 0.01% to about 1% by weight of the silicon dioxide.

Embodiment 41: The method of any one of embodiments 21-40, wherein the otic formulation has a viscosity between about 10 cP to about 10,000 cP, about 10 cP to about 5,000 cP, about 10 cP to about 1,000 cP, about 10 cP to about 500 cP, about 10 cP to about 250 cP, about 10 cP to about 100 cP, or about 10 cP to about 50 cP.

Embodiment 42: The method of any one of embodiments 21-41, wherein the otic formulation comprises between about 0.0001% to about 20% by weight of the growth factor, about 0.0001% to about 15% by weight of the growth factor, about 0.0001% to about 10% by weight of the growth factor, about 0.0001% to about 5% by weight of the growth factor, or about 0.0001% to about 1% by weight of the growth factor.

Embodiment 43: The method of any one of embodiments 21-42, wherein the otic formulation is free or substantially free of water, C1-C6 alcohols or C1-C6 glycols, C1-C4 alcohols or C1-C4 glycols, or any combination thereof.

Embodiment 44: The method of any one of embodiments 1-43, wherein the growth factor has a mean dissolution time of about 30 hours.

Embodiment 45: The method of any one of embodiments 1-44, wherein the growth factor is released from the formulation over a period of at least 3 days.

Embodiment 46: The method of any one of embodiments 1-45, wherein the growth factor is released from the formulation over a period of at least 4 days.

Embodiment 47: The method of any one of embodiments 1-46, wherein the growth factor is released from the formulation over a period of at least 5 days.

Embodiment 48: The method of any one of embodiments 1-47, wherein the growth factor is released from the formulation over a period of at least 7 days.

Embodiment 49: The method of any one of embodiments 1-48, wherein the growth factor is released from the formulation over a period of at least 14 days.

Embodiment 50: The method of any one of embodiments 1-49, wherein the growth factor is multiparticulate.

Embodiment 51: The method of any one of embodiments 1-50, wherein the growth factor is essentially in the form of micronized particles.

Embodiment 52: The method of any one of embodiments 1-50, wherein the growth factor is essentially in the form of nanosized particles.

Embodiment 53: The method of any one of embodiments 1-49, wherein the growth factor is essentially dissolved in the otic formulation.

Embodiment 54: The method of any one of embodiments 1-53, wherein the otic formulation is delivered by a drug delivery device selected from a needle and syringe, a pump, a microinjection device, a wick, a spongy material, and combinations thereof.

Embodiment 55: The method of any one of embodiments 1-54, further comprising an antioxidant.

Embodiment 56: The method of any one of embodiments 1-55, further comprising a mucoadhesive.

Embodiment 57: The method of any one of embodiments 1-56, further comprising a penetration enhancer.

Embodiment 58: The method of any one of embodiments 1-57, further comprising a preservative.

Embodiment 59: The method of any one of embodiments 1-58, further comprising a thickening agent or viscosity modulator agent.

Embodiment 60: The method of any one of embodiments 1-59, further comprising a chelator.

Embodiment 61: The method of any one of embodiments 1-60, further comprising an antimicrobial agent.

Embodiment 62: The method of any one of embodiments 1-61, further comprising a dye.

Embodiment 63: The method of any one of embodiments 1-62, further comprising cholesterol.

Embodiment 64: The method of embodiment 63, wherein the otic formulation comprises between about 0.01% to about 20% by weight of the cholesterol, about 0.01% to about 15% by weight of the cholesterol, about 0.01% to about 10% by weight of the cholesterol, about 0.01% to about 7% by weight of the cholesterol, about 0.01% to about 5% by weight of the cholesterol, about 0.01% to about 3% by weight of the cholesterol, about 0.01% to about 2% by weight of the cholesterol, or about 0.01% to about 1% by weight of the cholesterol.

Embodiment 65: The method of any one of embodiments 1-64, further comprising an excipient that increases the release rate of the therapeutic agent.

Embodiment 66: The method of any one of embodiments 1-64, further comprising an excipient that decreases the release rate of the therapeutic agent.

Embodiment 67: The method of any one of embodiments 1-66, for use in the treatment of an otic disease or condition associated with the outer, middle, and/or inner ear.

Embodiment 68: The method of embodiment 67, wherein the otic disease or condition associated with the outer, middle, and/or inner ear is hearing loss.

Embodiment 69: The method of embodiment 67 or embodiment 68, wherein the otic formulation repairs ribbon synapses.

Embodiment 70: The method of embodiment 42, wherein the otic formulation comprises between about 0.05% to about 0.5% by weight of the growth factor.

Embodiment 71: The method of embodiment 70, wherein the otic formulation comprises about 0.05% by weight of BDNF in Poloxamer 407.

Embodiment 72: The method of embodiment 70, wherein the otic formulation comprises about 0.5% by weight of BDNF in Poloxamer 407.

Embodiment 73: The method of any one of embodiments 1-72, wherein the otic formulation is formulated to provide sustained release of the growth factor into the inner ear to promote formation of synapses or repaire damaged synapses.

Embodiment 74: The method of any one of claims 1-73, wherein the otic formulation is administered through intratympanic injection.

Embodiment 75: The method of any one of claims 1-74, wherein the otic formulation is deposited on or close to the round window membrane of the subject.

Embodiment 76: The method of any one of claims 1-75, wherein the otic disease or condition is hearing loss or hearing impairment.

Embodiment 77: The method of any one of claims 1-76, wherein the synapses-affecting event is selected from the group consisting of: head trauma, traumatic brain injury (TBI), acoustic trauma, cochlear implant surgery, sudden sensorineural hearing loss, and drug-induced ototoxicity.

Embodiment 78: The method of claim 77, wherein the drug-induced ototoxicity is chemotherapy-induced ototoxicity.

Embodiment 79: The method of claim 78, wherein the drug-induced ototoxicity is cisplatin-induced ototoxicity.

Embodiment 80: The method of claim 77, wherein the drug-induced ototoxicity is antibiotic-induced ototoxicity.

Embodiment 81: The method of claim 80, wherein the antibiotic is selected from the group consisting of aminoglycoside antibiotics, macrolide antibiotics, and combinations thereof.

Embodiment 82: The method of any one of claims 1-81, wherein the otic formulation is administered within 13 day, within 12 days, within 11 day, within 10 days, within 9 days, within 8 days, within 7 days, within 6 days, within 5 days, within 4 days, within 3 days, within 2 days, or within 1 day after onset of the synapses-damaging event.

Embodiment 83: The method of any one of claims 1-81, wherein the otic formulation is administered within 7 days after onset of the synapses-damaging event.

Embodiment 84: The method of any one of claims 1-81, wherein the otic formulation is administered within 3 days after onset of the synapses-damaging event.

Embodiment 85: The method of any one of claims 1-81, wherein the otic formulation is administered within 1 day after onset of the synapses-damaging event.

### EXAMPLES

### Example A1— Effects of BDNF on Noise-Induced Hearing Loss In Vivo

In some forms of hearing loss, a reduction in auditory brainstem response (ABR) wave I amplitude and loss of synaptic ribbon synapses of the cochlear inner hair cells are common features. BDNF is known to provide trophic support to spiral ganglion neurons in the cochlea and to promote their survival and synaptic integrity. Rodent models of noise exposure can be used to test the in vivo efficacy of BDNF against hearing loss, specifically wave I amplitude reduction and synaptopathy.

### BDNF in Poloxamer 407

Poloxamer 407 gel at 16% was prepared using the cold method. In brief, a 16% w/w stock solution of poloxamer 407 was prepared by slowly adding it to a cold buffer solution (10 mM PBS, pH 7.4). Sterilization was achieved by filtration. Human recombinant BDNF was suspended with an appropriate amount of poloxamer 407 solution to reach a range of concentration of 0.05 to 5.0 mg/ml BDNF.

### Noise-Induced Hearing Loss/Synaptopathy

Male rats (Charles River) approximately 10-11 months of age served as subjects (N=6 per group). Prior to any procedures, animals were anesthetized using a combination of xylazine (10 mg/kg) and ketamine (90 mg/kg) for up to one hour via the intraperitoneal route. If needed, an intraoperative booster was administered intraperitoneally representing one-tenth of the original dose.

Noise exposure—Anesthetized animals (4 at a time) were placed in an enclosure and exposed to noise. The characteristics of the acoustic trauma were as follows: a single period of 1 h, sound pressure level of 105 dB SPL with a narrow band pass of 8-16 kHz. Noise was delivered using a Grason Stadler white noise generator, filtered through a Krohn Hite 3750 unit, amplified using a Crown D75 unit, and conveyed via a JBL speaker (model 2446H). Following treatment, the animals were returned to the vivarium. Noise exposure was calibrated over a range of 80-115 dB SPL across a wide range of frequencies using the Acoustical Interface Precision Microphone System and the Tucker Davis Technologies system.

Intratympanic injection—Each animal was positioned so that the head was tilted at an angle to favor injection towards the round window niche. Briefly, under visualization with an operating microscope, 20 μL of the formulation was injected using a 25G (Gauge) 11/2 inch needle through the tympanic membrane into the superior posterior quadrant. Formulations were delivered using a perfusion pump at the rate of 2 μL/sec. Contact with the round window membrane was maintained for 30 minutes by placing the animal in a recumbent position. During the procedure and until recovery, animals were placed on a temperature controlled (40° C.) heating pad until consciousness was regained at which time they were returned to the vivarium.

Auditory Brainstem Response (ABR) assessment (wave 1)—During the procedure, additional anesthetic (xylazine and ketamine) was administered if needed to maintain the depth of anesthesia sufficient to insure immobilization and relaxation. ABRs were recorded in an electrically and acoustically shielded chamber, one ear at a time. Needle electrodes were placed at the vertex (active) and immediately below the pinna of the test ear (reference) and contralateral ear (ground). Tucker Davis Technologies (TDT) System III hardware and SigGen/BioSig software (TDT) were used to present the stimulus and record the ABR responses. Tones were delivered through a Tucker-Davis open-field ES1 driver placed 5 cm above the animal's ear. Acoustic calibration was performed with TDT software (SigCal) and thresholds were expressed as dB SPL in conditions identical to that of threshold recordings in animals. Stimulus presentation (15 ms tone bursts, with 1 ms rise/fall times) were presented 10 per second. Up to 512 responses were averaged for each stimulus level. Responses were collected for stimulus levels in 5 dB decrement steps at 4 frequencies: 4 kHz, 10 kHz, 20 kHz and 40 kHz. Thresholds were interpolated between the lowest stimulus level where a response was observed, and 5 dB lower, where no response was observed. The threshold was then reported as the mean value between these two stimuli conditions. Wave 1 was quantified by averaging the wave 1 amplitude from intensities ranging between 60-80 dB SPL.

Tissue collection—At 28 days post-IT injection, animals were sacrificed as follows. Animals were anesthetized with a combination of xylazine (10 mg/kg) and ketamine (90 mg/kg). Anesthetized animals were then given a lethal dose of anesthesia via cardiac puncture. Once dead, animals were immediately decapitated, the temporal bones were isolated, and the middle ear bullae were opened to expose the cochleae. The cochleae were then perfused directly with fixative as follows. A small gauge needle was used to puncture the round window membrane and the bone within the oval window of the cochlea. A small hole was drilled into the apical turn of the cochlea, and 1-2 mls of cold PFA were slowly perfused through the apical hole. Perfused cochleae were then immersed in 4% PFA and post-fixed for 2-6 hrs. Cochleae were then rinsed in PBS and stored in fresh PBS at 4° C.

Cochlear Decalcification/Dissection—Fixed cochleae were decalcified in a large volume of 10% EDTA in PBS at room temperature with mild shaking for 72 hours. After decalcification, samples were rinsed in PBS, then carefully dissected to remove the residual bone from the tissue so that just the cochlear duct attached to the modiolar core remained. Dissected tissue was then permeabilized in 0.5% PBS-Triton (PBS-T) at room temperature for a minimum of 3 hours.

Immunohistochemistry—Fixed and dissected cochleae were incubated in PBS-T with 10% normal goat serum with the following primary antibodies overnight at 4° C. with nutation: CtBP2 (1:250; mouse monoclonal IgG1, BD Biosciences), GluR2 (1:2000; mouse monoclonal IgG2a, Millipore), MyosinVIIa (1:1000; rabbit polyclonal, Proteus Biosciences). Cochleae were then rinsed in PBS-T and incubated in the appropriate fluorescent secondary antibodies (1:1000; anti-mouse IgG1-546, anti-mouse IgG2a-488, anti-rabbit IgG-633) in PBS-T with 10% goat serum for 2 hrs at room temperature. Cochleae were then rinsed in PBS, stained with DAPI to label nuclei then finely dissected in PBS and flat-mounted on glass slides with anti-fade mounting medium (Southern Biotech).

Synaptic Analysis—Flat-mounted cochlear pieces from each ear were imaged at low magnification (10×) and the total length of the imaged tissue was measured and the frequency domains (4, 10, 20 and 40 kHz) were mapped onto the images. High-magnification confocal Z-stack images were then acquired using a Zeiss LSM880 laser scanning confocal microscope at each specific frequency at a minimum of 0.35 μm steps such that the inner hair cells were fully imaged from their basal pole to the cuticular plate. Images included at least 10 contiguous inner hair cells per frequency domain. The number of aligned pre/post-synaptic puncta was then counted for each inner hair cell per region and the average number of puncta per inner hair cell per frequency domain was calculated.

### Study Design

Animals were exposed to noise on Day 0. On Day 1 (24 hrs later) animals received a single unilateral intratympanic administration of vehicle (Poloxamer 407) or the BDNF formulation (0.005% BDNF in Poloxamer 407). ABR threshold and ABR wave I amplitude were measured weekly in live animals for up to 4 weeks. Synaptic counts were performed on processed tissue obtained from animals sacrificed on Day 28. Naïve animals were not exposed to noise, nor did they receive an intratympanic injection.

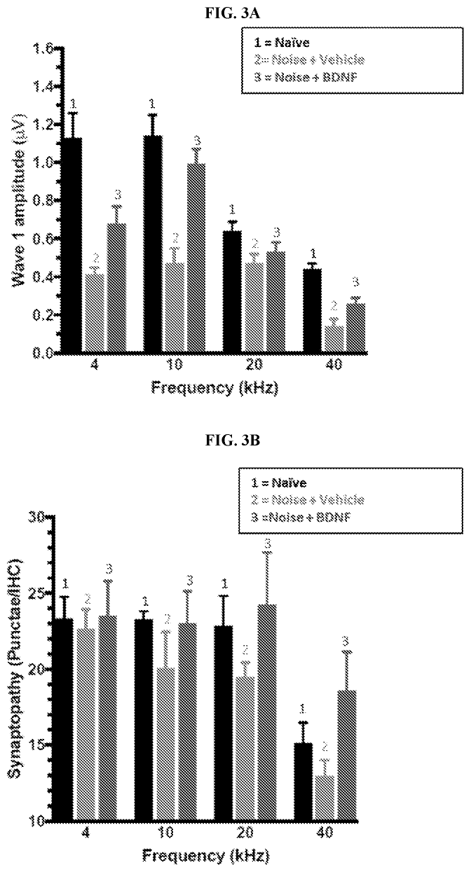

FIG. 2 shows the visualization of synapses on hair cells. FIG. 3A shows that treatment with sustained release BDNF formulations leads to improved hearing function (wave I amplitude) in noise exposed adult rats compared to vehicle treatment. FIG. 3B shows that treatment with sustained release BDNF formulations leads to an increased number of ribbon synapses per inner hair cell in noise exposed adult rats compared to vehicle treatment.

### Example A2— Sustained Release of BDNF in Poloxamer and MCT Formulations

### BDNF in Poloxamer 407

Poloxamer 407 solution at 32% was prepared by slowly adding it to a cold buffer solution (50 mM tris buffer with saline, pH 7.4). Sterilization was achieved by filtration. Human recombinant BDNF was either diluted or concentrated with the same tris buffer solution to a concentration twice the target concentration. The solutions were sterilized by filtration. Combining the above two solutions at 1:1 ratio yields a solution or a suspension containing 16% P407 and a concentration range of 0.005% to 0.5% BDNF.

### BDNF/PVP (K25) in MCT SiO2

BDNF/PVP (2% or 10%) in MCT/SiO2 (0.5%) formulations were prepared as follows. PVP was dissolved in water for injection at 2 or 10% concentration. The BDNF frozen solution was thawed at 5° C. overnight and was then diluted with water for injection to a concentration of 0.15% BDNF. Combining the above two solutions at a ratio of 1:1 (w/w) yields a solution with 0.075% BDNF and 1% or 5% PVP. The solution was sterile filtered via a 0.22 um filter, filled into vials and then lyophilized. The dry powder was then dispersed into sterile filtered MCT by bead-blasting. Finally, the heat sterilized SiO2 was added. The final suspension contains 0.15% BDNF, 2 or 10% PVP and 0.5% SiO2 in MCT.

### BDNF/Carbopol (971P) in MCT/SiO2

BDNF/Carbopol (0.15%) in MCT/SiO2 (0.5%) formulation was prepared as follows. Carbopol was dissolved in water for injection at 0.04% concentration. The BDNF frozen solution was thawed at 5° C. overnight and was then diluted with water for injection to a concentration of 0.04% BDNF. Combining the above two solutions at a ratio of 1:1 (w/w) yields a solution with 0.02% BDNF and 0.02% Carbopol. The solution was sterile filtered via a 0.22 um filter, filled into vials and then lyophilized. The dry powder was then dispersed into sterile filtered MCT by bead-blasting. Finally, the heat sterilized SiO2 was added. The final suspension contains 0.15% BDNF, 0.15% Carbopol and 0.5% SiO2 in MCT.

### BDNF/Poloxamer (P407) in MCT/SiO2

BDNF/P407 (10%) in MCT/SiO2 (0.5%) formulation was prepared as follows. P407 was dissolved in water for injection at10% concentration. The BDNF frozen solution was thawed at 5 C overnight and was then diluted with water for injection to a concentration of 0.15% BDNF. Combining the above two solutions at a ratio of 1:1 (w/w) yields a solution with 0.075% BDNF and 5% P407. The solution was sterile filtered via a 0.22 um filter, filled into vials and then lyophilized. The dry powder was then dispersed into sterile filtered MCT by bead-blasting. Finally, the heat sterilized SiO2 was added. The final suspension contains 0.15% BDNF, 10% P407 and 0.5% SiO2 in MCT.

### Pharmacokinetics

Female rats (Charles River) weighing 200-300 g of approximately 12-16 weeks of age served as subjects (N=4 per group). Prior to any procedures, animals were anesthetized using a combination of xylazine (10 mg/kg) and ketamine (90 mg/kg) for up to an hour via the intraperitoneal route. If needed, an intraoperative booster was administered intraperitoneally representing a one-tenth of the original dose.

Intratympanic injection—Each animal was positioned so that the head was tilted at an angle to favor injection towards the round window niche. Briefly, under visualization with an operating microscope, 20 μL of the formulation was injected using a 25G (Gauge) 11/2 needle through the tympanic membrane into the superior posterior quadrant. Formulations were delivered using a perfusion pump at the rate of 2 μL/sec. Contact with the round window membrane was maintained for 30 minutes by placing the animal in a recumbent position. During the procedure and until recovery, animals were placed on a temperature controlled (40° C.) heating pad until consciousness was regained at which time they were returned to the vivarium.

Perilymph sampling procedure—The skin behind the ear of anesthetized rats was shaved and disinfected with povidone-iodine. An incision was then made behind the ear, and muscles were carefully retracted from over the bulla. A hole was drilled through the bulla using a dental burr so that the middle ear was exposed and accessed. The cochlea and the round window membrane were visualized under a stereo surgical microscope. The basal turn of bulla was cleaned by using small cotton ball. A unique microhole was hand drilled through the bony shell of the cochlea (cochlear capsule) adjacent to the round window. Perilymph (about 2 μL) was then collected using a microcapillary inserted into the cochlear scala tympani. Perilymph samples were added to a vial containing 18 μL of acetonitrile/water (50/50, v/v), stored at −80° C. until analysis.

### Analytical Method

Concentrations of BDNF in perilymph and samples were determined using commercially available ELISA kits. The limits of detection of human BDNF were 4 pg/mL.

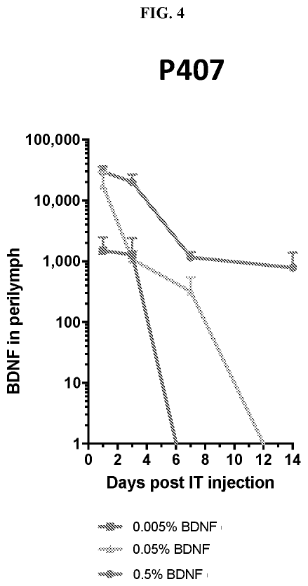

FIG. 4 shows the perilymph concentrations of BDNF following a single IT injection in rats in poloxamer 407 formulation. BDNF was administered at the indicated doses. Perilymph BDNF is in arbitrary units.

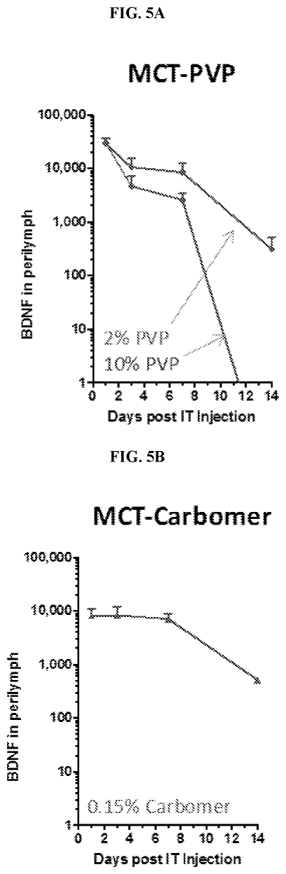

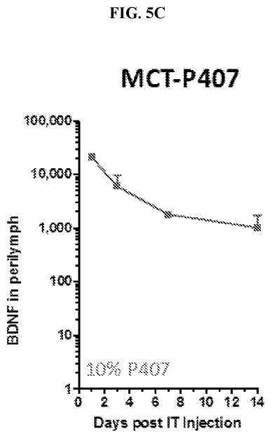

FIGS. 5A, 5B, and 5C show perilymph concentrations of BDNF following a single IT injection in rats in MCT formulations. BDNF was administered at a dose of 0.15% (1.5 mg/ml) in the MCT formulation containing different polymers: PVP (polyvinylpyrrolidone) at 2 or 10% (FIG. 5A); 0.15% carbomer (FIG. 5B); or P407 (poloxamer 407) at 10% (FIG. 5C). Perilymph BDNF is in arbitrary units.

### Example B1—Preparation of a Thermoreversible Gel Formulation

TABLE A
Thermoreversible Gel Growth Factor Otic Formulation

| | Concentration in 1000 mL | |
|---|---|---|
| Ingredient | aqueous solution | |
| Growth Factor | 0.001-10 | (wt %) |
| Polyoxyethylene-polypropylene | 14-21 | (wt %) |
| triblock copolymer | | |
| (e.g. Poloxamer 407) | | |

| | |
|---|---|
| pH adjusting agent | q.s. for pH = 5.5-8.0 |
| (e.g. HCl) | |
| Sterile water | q.s. to 1000 mL |

An exemplary batch of gel formulation containing, for example, 1.5% of a growth factor described herein is prepared by dissolving Poloxamer 407 (BASF Corp.) in 50 mM Tris buffer and 77 mM NaCl solution with a pH between 5.5-8.0. The appropriate amount of the growth factor is added and the formulation is mixed until a homogenous suspension is produced. The mixture is maintained below room temperature until use.

### Example B2— In Vitro Comparison of Gelation Temperature

The effect of Poloxamer 188 and any one of the growth factors described herein on the gelation temperature and viscosity of Poloxamer 407 formulations is evaluated with the purpose of manipulating the gelation temperature. In some embodiments, the growth factor is BDNF.

A 25% Poloxamer 407 stock solution in PBS buffer and Poloxamer 188NF from BASF are used. An appropriate amount of the growth factor is added to the solutions described in Table B to provide a 2% formulation of the growth factor.

A PBS buffer (pH 7.3) is prepared by dissolving 805.5 mg of sodium chloride (Fisher Scientific), 606 mg of sodium phosphate dibasic anhydrous (Fisher Scientific), 247 mg of sodium phosphate monobasic anhydrous (Fisher Scientific), then QS to 200 g with sterile filtered DI water.

TABLE B
Preparation of Samples Containing Poloxamer 407/Poloxamer 188

| | 25% P407 | | |
|---|---|---|---|
| | Stock Solution | Poloxamer 188 | PBS Buffer |
| Sample | (g) | (mg) | (g) |
| 16% P407/10% P188 | 3.207 | 501 | 1.3036 |
| 17% P407/10% P188 | 3.4089 | 500 | 1.1056 |
| 18% P407/10% P188 | 3.6156 | 502 | 0.9072 |
| 19% P407/10% P188 | 3.8183 | 500 | 0.7050 |
| 20% P407/10% P188 | 4.008 | 501 | 0.5032 |
| 20% P407/5% P188 | 4.01 | 256 | 0.770 |

Gelation temperature of the above formulations are measured using procedures described herein.

An equation is fitted to the data obtained and is utilized to estimate the gelation temperature of F127/F68 mixtures (for 17-20% F127 and 0-10% F68).

T_{gel}=−1.8(% F127)+1.3(% F68)+53

An equation is fitted to the data obtained and can be utilized to estimate the Mean Dissolution Time (hr) based on the gelation temperature of F127/F68 mixtures (for 17-25% F127 and 0-10% F68), using results obtained in examples above:

MDT=−0.2(T_{gel})+8.

### Example B3—Preparation of Medium Chain Triglyceride Formulations

Formulations 1, 2, and 3 are prepared with the appropriate amounts of a growth factor and medium chain triglycerides (CRODAMOL, GTCC-LQ-(MV), PhEur) as shown in the below table (Table C).

The formulations are prepared by adding the target weight percentage of any one of the growth factors described herein to the appropriate amount of medium chain triglyceride for a total volume of about 100 mL. The formulations are mixed until complete dissolution. The formulations are then sterilized by passing the formulations through 0.22 μm sterilizing grade filters under aseptic conditions. The sterilized solutions are then filled into vials or pre-filled syringes, which were then used to test the formulations.

TABLE C

| Component | Formulation 1 | Formulation 2 | Formulation 3 |
|---|---|---|---|
| Growth Factor | 0.01 wt % | 0.15 wt % | 0.5 wt % |
| CRODAMOL, | QS to 100 mL | QS to 100 mL | QS to 100 mL |
| GTCC-LQ-(MV), | | | |
| PhEur | | | |

### Example B4— Additional Preparation of Medium Chain Triglyceride Formulations—Growth Factor

Formulations 4, 5, and 6 are prepared with the appropriate amounts of the growth factor and medium chain triglyceride (CRODAMOL, GTCC-LQ-(MV), PhEur) as shown in the below table (Table D).

The formulations are prepared by adding the target weight percentage of the growth factor to the appropriate amount of medium chain triglyceride. The formulations are mixed until complete dissolution. The formulations are then sterilized by passing the formulations through 0.22 μm sterilizing grade filters under aseptic conditions. The sterilized solutions are then filled into vials or pre-filled syringes, which are then used to test the formulations.

TABLE D

| Component | Formulation 4 | Formulation 5 | Formulation 6 |
|---|---|---|---|
| Growth Factor | 1 wt % | 5 wt % | 10 wt % |
| CRODAMOL, | QS to 100 mL | QS to 100 mL | QS to 100 mL |
| GTCC-LQ-(MV), | | | |
| PhEur | | | |

### Example B5—Preparation of Medium Chain Triglyceride Formulations

Medium chain triglyceride formulations are prepared with the appropriate amount of growth factor, medium chain triglyceride, and viscosity modulating agents as shown in the below tables (Tables E-H). In some embodiments, the formulations further comprise cholesterol as shown in the below tables (Tables I-L). Also in some instances, the medium chain triglyercide as shown in below tables is replaced with a mixture of mixture of long-chain triglyceride and medium-chain triglycerides (0.1:99.9 to 99.9:0.1),

### Growth Factor Solution in MCT

The formulation is prepared by dissolving the appropriate amount of a growth factor and one or more than one of the viscosity modulating agents, such as PVP, carbomer, and P407, in water for injection and sterile filtering the solution. The sterilized solution is lyophilized to form the dry cake. The appropriate amount of the dry cake is aseptically added to the appropriate amount of sterile filtered medium chain triglyceride. The formulation is mixed until a uniform suspension is achieved. If needed, the suspension is homogenized to reduce the particle size to below 10 microns (D50). Then the appropriate amount of sterilized silicon dioxide is added to the suspension, if needed. The final formulation is mixed until a uniform suspension is achieved and then is filled into vials.

### Growth Factor Suspension in MCT and SiO2

The formulation is prepared by adding the target weight percentage of a growth factor that has been micronized and gamma irradiated to the appropriate amount of medium chain triglyceride that has been sterilized via filtration. The formulation is mixed until a uniform suspension is formed. The appropriate amount of SiO2 is then added and is mixed until uniform. The resulting uniform suspension is then filled into vials.

### Growth Factor Nano-Suspension in MCT and SiO2

The formulation is prepared by adding the target weight percentage of a growth factor that has been micronized and gamma irradiated to the appropriate amount of medium chain triglyceride that has been sterilized via filtration. The formulation is mixed until a uniform suspension is formed. Ball milling equipment is then used to reduce the particle size to below 0.2 μm. The appropriate amount of SiO2 is then added and is mixed until uniform. The resulting uniform suspension is then filled into vials.

TABLE E

| Component | Amount (wt %) |
|---|---|
| Growth Factor | 0.0001%-10% |
| Medium Chain Triglyceride | QS to 100 mL |

TABLE F

| Component | Amount (wt %) |
|---|---|
| Growth Factor | 0.0001%-10% |
| Medium Chain Triglyceride | QS to 100 mL |
| SiO2 | 0.01%-10% |

TABLE G

| Component | Amount (wt %) |
|---|---|
| Growth Factor | 0.0001%-10% |
| Medium Chain Triglyceride | QS to 100 mL |
| PVP, P407, or carbomer | 0.01-20% |

TABLE H

| Component | Amount (wt %) |
|---|---|
| Growth Factor | 0.0001%-10% |
| Medium Chain Triglyceride | QS to 100 mL |
| SiO2 | 0.01%-10% |
| PVP, P407, or carbomer | 0.01%-20% |

TABLE I

| Component | Amount (wt %) |
|---|---|
| Growth Factor | 0.0001%-10% |
| Cholesterol | 0.01%-20% |
| Medium Chain Triglyceride | QS to 100 mL |

TABLE J

| Component | Amount (wt %) |
|---|---|
| Growth Factor | 0.0001%-10% |
| Medium Chain Triglyceride | QS to 100 mL |
| SiO2 | 0.01%-10% |
| Cholesterol | 0.01%-20% |

TABLE K

| Component | Amount (wt %) |
|---|---|
| Growth Factor | 0.0001%-10% |
| Medium Chain Triglyceride | QS to 100 mL |
| PVP, P407, or carbomer | 0.01-20% |
| Cholesterol | 0.01%-20% |

TABLE L

| Component | Amount (wt %) |
|---|---|
| Growth Factor | 0.0001%-10% |
| Medium Chain Triglyceride | QS to 100 mL |
| SiO2 | 0.01%-10% |
| PVP, P407, or carbomer | 0.01%-20% |
| Cholesterol | 0.01%-20% |

### Example C—Survival of Spiral Ganglion Neurons

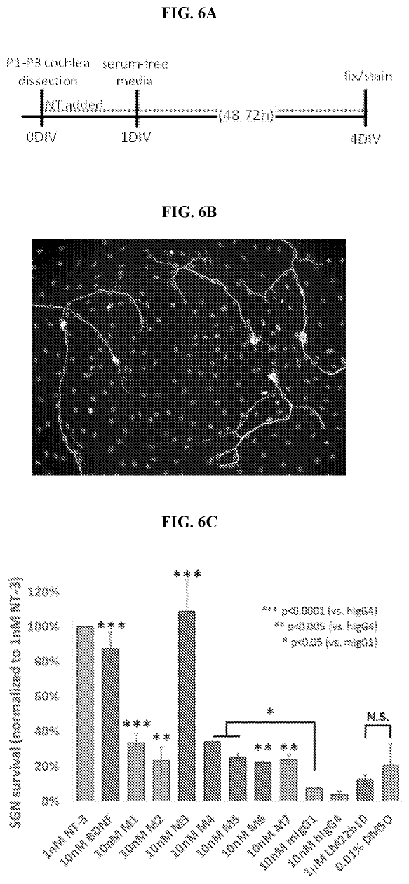

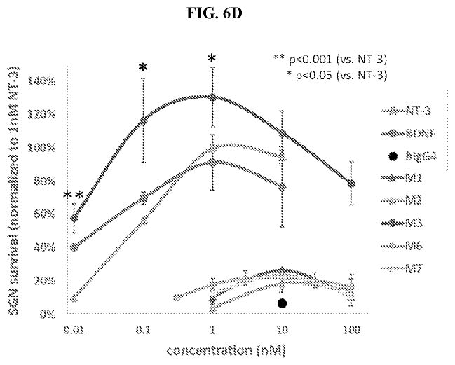

Spiral ganglion neurons (SGNs) were harvested from P1-P3 rates, dissociated, and cultured in 96-well plates. The SGNs were treated with neurotrophins and/or antibodies at various concentrations. After about 24 hours, media was exchanged to serum-free media and the neurotrophins and/or antibodies were replenished. Cells were cultured for an additional 48-72 hours before being fixed using 4% paraformaldehyde (PFA) and stained using 4′,6-diamidino-2-phenylindole (DAPI). FIG. 6A schematically depicts this experimental scheme, while FIG. 6B shows neuronal cell bodies and neurites stained with neurofilament (green) and nuclei stained with DAPI. Surviving neurons were identified as cells with intact soma and at least one neurite.

FIG. 6C shows SGN survival normalized to 1 nanomolar (nM) of NT-3 for treatment with neurotrophins and antibodies at various concentrations including 1 nM NT-3, 10 nM BDNF, 10 nM M1, 10 nM M2, 10 nM M3, 10 nM M4, 10 nM M5, 10 nM M6, 10 nM M7, 10 nM mIgG1, 10 nM hIgG4, 1 μM LM22b10, and 0.01% DMSO. As shown in the figure, the number of surviving SGNs was increased when cultured with neurotrophins or M-antibodies, compared to when treated with mouse IgG1 and human IgG4 iso-type controls. Survival of SGNs was not improved with LM22b10 compared to a vehicle-only control, 0.01% DMSO.

FIG. 6D shows SGN survival normalized to 1 nM of NT-3 for treatment with neurotrophins and antibodies at various concentrations. As shown in the figure, normalized SGN survival was comparatively lower for M1, M2, M6, and M7 at all concentrations.

### Example D—TrkB/TrkC Agonists Promote SGN Complexity (Dissociated SGNs)

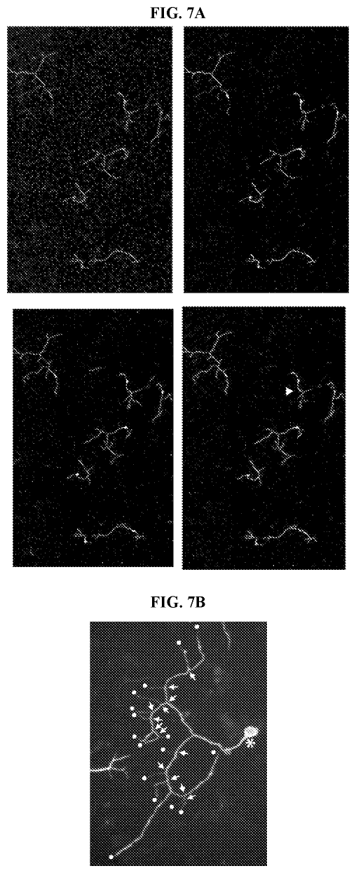

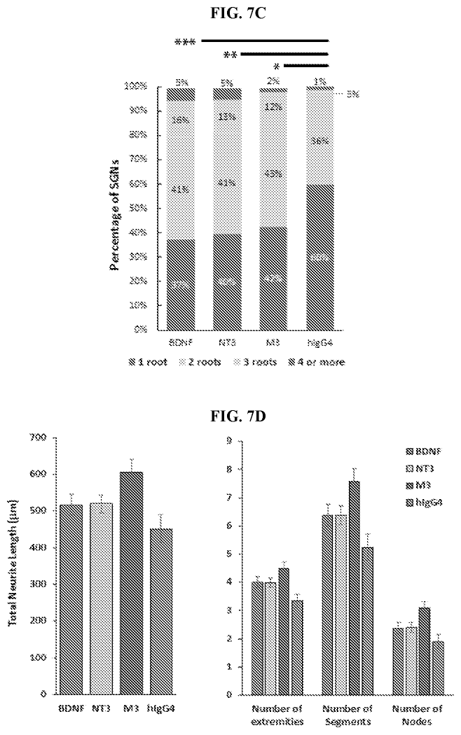

SGNs were immunostained, imaged, and batch analyzed with PerkinElmer Harmony 4.6 software. Changes in the complexities of SGNs upon treatment with various agents were examined. FIG. 7A shows SGNs immunostained for neurofilament and DAPI and imaged at 20×. All of the images in a single well were stitched together and analyzed. SGN somas are shown in the upper right panel and SGN neurites are shown in the lower left panel. As shown in the lower right panel, each neurite tree is assigned to a single cell for analysis. Depending on the stitching, occasionally neurites will not meet the criteria due to gaps that exceed a user-defined threshold. FIG. 7B shows roots (indicated with asterices), extremities (indicated with white dots), nodes (indicated with arrows), segments (indicated with discrete sections of neurite between nodes or extremities), and total neurite length measured for each neuron. FIG. 7C shows the percentage of SGNs having various numbers of roots upon treatment with 10 nM BDNF, 1 nM NT-3, or 1 nM M3 compared to the IgG-treated control (*p<0.05, **p<0.005, and p<0.001). FIG. 7D shows total neurite length (left panel) as well as total number of extremities, segments, and nodes per cell upon treatment with BDNF, NT-3, or M3. These differences were not statistically significant.

### Example E—Neurite Outgrowth (SGN Explants)

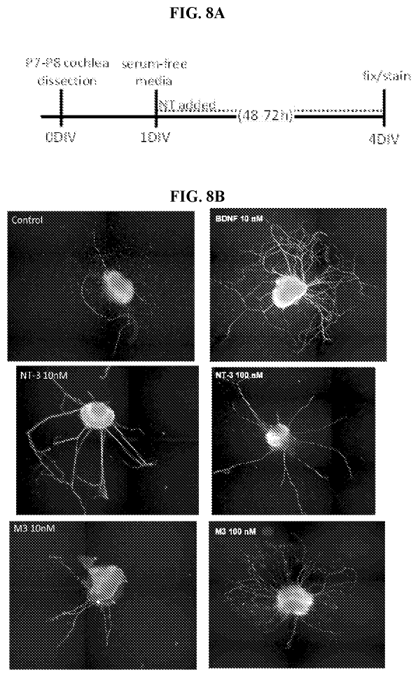

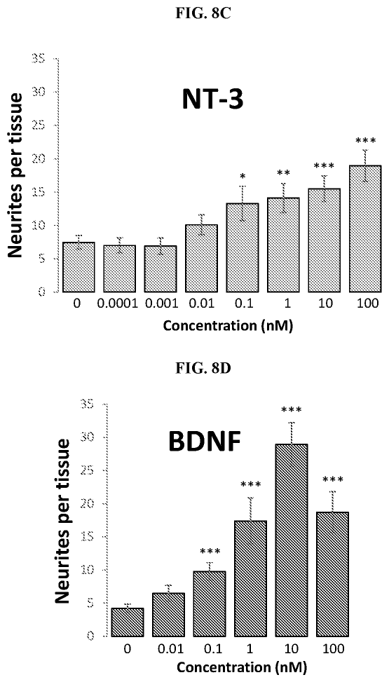

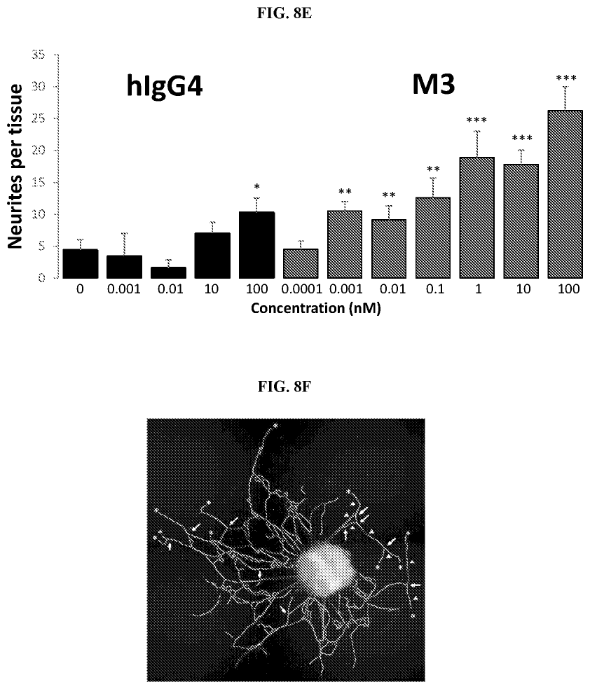

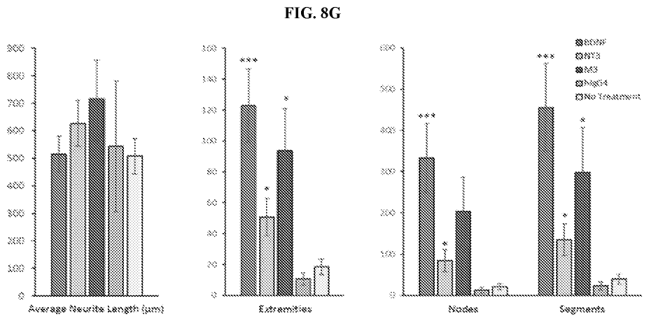

Treatment with various agents may promote neurite extension in SGN explants. SGNs were harvested from P7-8 rats, cut into ˜1 millimeter (mm) sections and cultured in 96-well plates coated with human fibronectin. After about 24 hours, media was exchanged to serum-free media, and neurotrophins and/or antibodies were added at various concentrations. Tissues were cultured for an additional 48-72 hours and then fixed with 4% PFA and immunostained. FIG. 8A schematically depicts this experimental scheme, while FIG. 8B shows stained neurofilaments for samples treated with no neurotrophin/antibody (upper left image), 10 nM BDNF (upper right image), 10 nM NT-3 (middle left image), 100 nM NT-3 (middle right image), 10 nM M3 (lower left image), and 100 nM M3 (lower right image).

FIGS. 8C and 8D show neurites per tissue at various concentrations of NT-3 (FIG. 8C) and BDNF (FIG. 8D). FIG. 8E shows neurites per tissue at various concentrations of hIgG4 (left) and M3 (right). In FIGS. 8C-8E, * indicates p<0.05, ** indicates p<0.005, and *** indicates p<0.0005 vs untreated.

As shown in FIG. 8F, explants were imaged and analyzed individually using Harmony analysis software to examine neurite length, nodes (arrows), segments (arrowheads), and the number of extremities (asterices). FIG. 8G shows the effect of BDNF, NT-3, and M3 on neurite complexity of SGN explants compared to IgG-treated or untreated controls.

### Example F—Synaptopathy Model (Cochlear Explants)

Cochlear synaptopathy is a primary pathology of various otic insults, including aggeg-related hearing loss. It is characterized by the loss of afferent synapses, the synaptic connections between inner hair cells and type I spiral ganglion neurons. Cochlear synaptopathy in humans has been implicated in “hidden hearing loss,” i.e., speech-in-noise hearing difficulties.

Neurotrophins such as BDNF and NT-3 and their mimetics establish afferent synapses during development, and disruption of BDNF, NT-3, or their receptors (TrkB and TrkC) affects afferent synapses and hearing function.

Neurotrophin and/or antibody treatment may be combined with excitotoxin treatment. An excitotoxin is a chemical such as an amino acid that overstimulates neuron receptors, causing impulses to be fired at such a rate as to exhaust the neuron receptors.

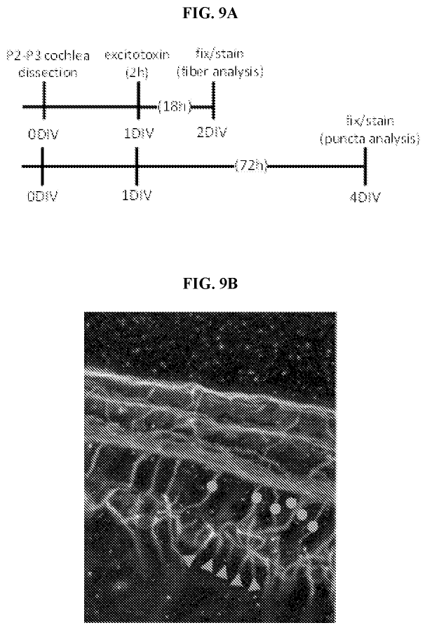

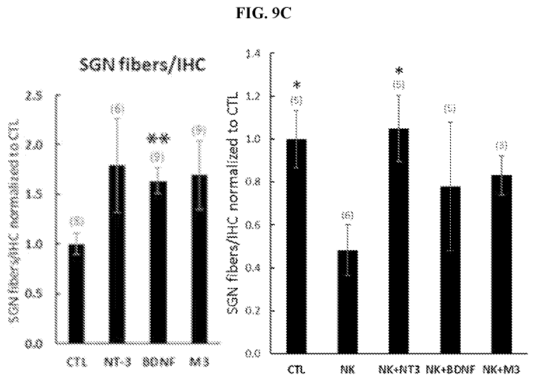

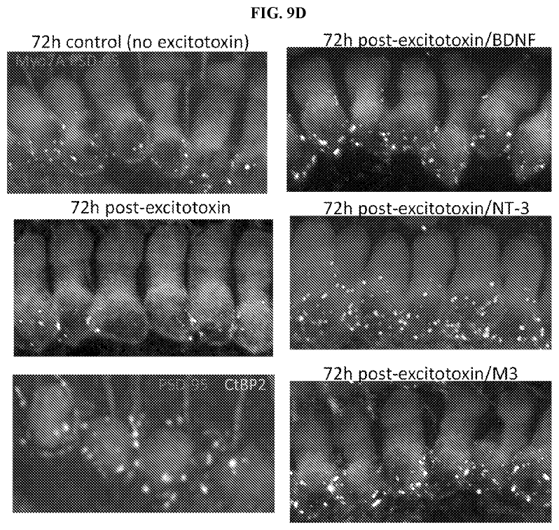

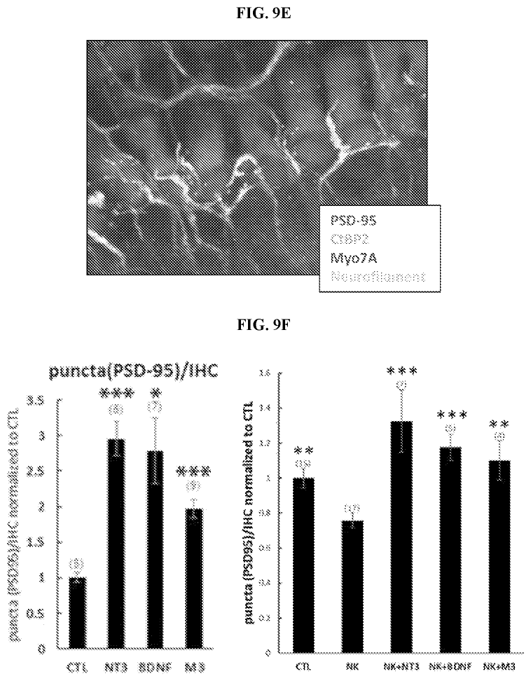

Restoration of SGN fibers. P2-P3 rat cochleae were established and then exposed to excitotoxin (NK: 0.5 mM NMDA+0.5 mM kainate) for about 2 hours. Neurotrophins and/or antibodies (e.g., Trk agonists) were added in various concentrations after excitotoxin was removed. Explants were cultured for an additional 18 or 72 hours before being fixed with 4% PFA and immunostained. FIG. 9A schematically depicts this experimental scheme. As shown in FIG. 9B, type I SGNs were estimated by counting the number of fibers approaching inner hair cells (IHCs; triangles) and subtracting the number of fibers passing through to the outer hair cells (OHCs; circles). FIG. 9C shows SGN fibers/IHC normalized to CTL for various agents for no excitotoxin and 72 hour culture (left panel) and excitotoxin treatment and 18 hour culture (right panel). Trk agonists increased the number of fibers in explants.

FIG. 9D shows images of cochlear explants taken under various conditions. The upper left image corresponds to a control that is not treated with excitotoxin. The middle left image corresponds to a sample 72 hours post-excitotoxin treatment. The upper, middle, and lower right images correspond to samples 72-hours post excitotoxin and BDNF, NT-3, or M3 treatment, respectively. The lower left image shows post-synaptic density protein 95 (PSD-95) and C-terminal-binding protein 2 (CtBP2).

Restoration of synapses. Cochleae were exposed to excitotoxin and Trk agonists, as above, and then cultured for 72 hours. Synapses were stained for PSD-95, imaged, and counted. Trk agonists increased the number of synaptic puncta and restored them to control levels after excitotoxic insult.

FIG. 9E shows an image of cochlear explants with PSD-95, CtBP2, neurofilament (lines), and Myo7a. FIG. 9F shows puncta (PSD95)/IHC normalized to CTL for various Trk agonists for no excitotoxin and 72 hour culture (left panel) and excitotoxin and 72 hour culture (right panel) (*p<0.05 (CTL vs. NK)).

### Example G—Inner Ear Pharmacokinetics of BDNF in P407

Animals: Male Sprague-Dawley rats (5-6 months of age) were used for efficacy studies and females (2-3 months) were used for pharmacokinetic studies.

Formulations: Human recombinant BDNF was suspended in the thermoreversible polymer Poloxamer 407 (16%) to reach a range of BDNF concentrations.

Intratympanic (IT) injection: Formulations were administered under anesthesia as a single IT injection (20 μl) through the tympanic membrane into the middle ear, targeting the round window membrane (RWM).

Inner ear pharmacokinetics: Animals (n=4 per time point) received a single unilateral intratympanic injection of the different treatment doses. BDNF levels in the perilymph were determined at the indicated times by ELISA.

Noise-induced cochlear synaptopathy: Rats (n=44 per treatment group) received a single acoustic trauma (105 dB SPL, 8-16 kHz, 1 h) under anesthesia. 24 hours later (Day 1) animals received a single bilateral IT injection of the treatment regimens. Auditory function (ABR and ABR wave 1 amplitude) was monitored at day 1 and then every 7 days for 28 days. At termination (Day 28), cochleae were collected and assessed for evidence of cochlear synaptopathy (IHC synaptic puncta counts).

Immunohistochemistry: Dissected cochleae were immunostained with antibodies against presynaptic (CtBP2) and postsynaptic (GluR2) proteins and MyosinVIIa to label hair cells. Cochleae were then frequency mapped and high-magnification images were obtained with a Zeiss LSM-880 confocal microscope and imaged at an interval of 0.35 μm.

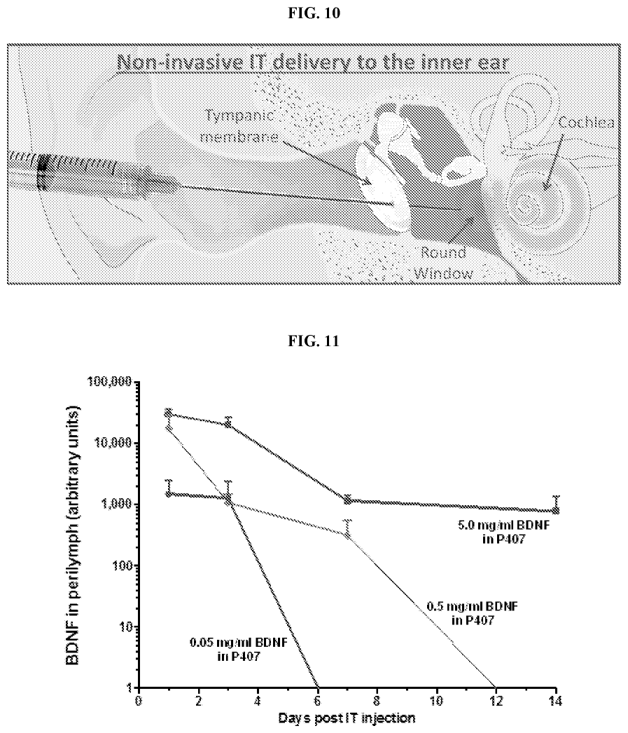

FIG. 10 schematically illustrates non-invasive intratympanic (IT) delivery of an agent such as BDNF to the inner ear.

BDNF levels in the cochlear perilymph. Adult rats received a unilateral intratympanic injection of 20 μl of BDNF/P407 formulation on Day 0. Perilymph was collected from the base of the cochlea via cochleostomy at days 1, 3, 7, and 14 and BDNF levels were quantified by ELISA. FIG. 11 shows BDNF in perilymph (pg/ml) at various times post IT injection for various concentrations of BDNF in P407.

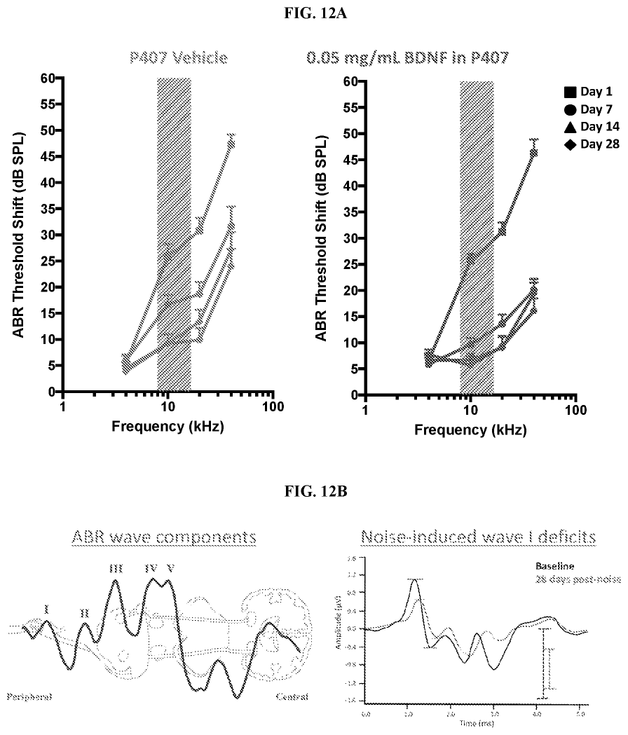

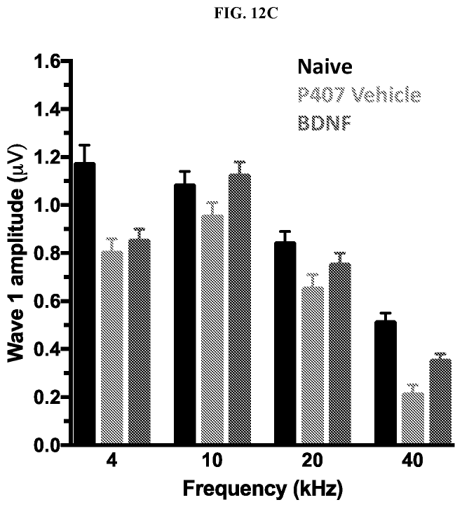

Intratympanic BDNF improves Auditory Brainstem Response (ABR) thresholds and wave I amplitudes in adult rats. The Auditory Brainstem Response (ABR) is an objective test that measures hearing sensitivity and can identify abnormalities in the peripheral auditory system and the auditory pathway up through the brainstem. Adult male rats were bilaterally exposed to noise followed by an intratympanic injection of 0.05 mg/ml BDNF in P407 24 hours after noise exposure. ABR recordings were obtained at days 1, 7, 14, and 28. BDNF-treated animals showed a more rapid recovery of initial threshold shifts and overall reduced levels of hearing loss. FIG. 12A shows ABR threshold shift (dB SPL) at various frequencies for P407 vehicle alone and 0.05 mg/ml BDNF in P407.

The ABR Wave I amplitude is an indicator of the strength of the distal cochlear nerve signal, i.e., the response of the spiral ganglion neurons/synapses. FIG. 12B shows ABR wave components (left panel) and noise-induced wave I deficits (right panel). Wave I amplitude shift at 10 kHz in the same ear at baseline prior to noise exposure (black line) and 28 days post-noise exposure. The inset dashed lines highlight the significant reduction in wave I after noise exposure. As shown in FIG. 12C, ABR wave I amplitudes were measured in adult male rats after BDNF or vehicle treatment following noise exposure or in naive undamaged ears. Significant improvements in wave I amplitudes were detected in BDNF-treated animals across many frequencies.

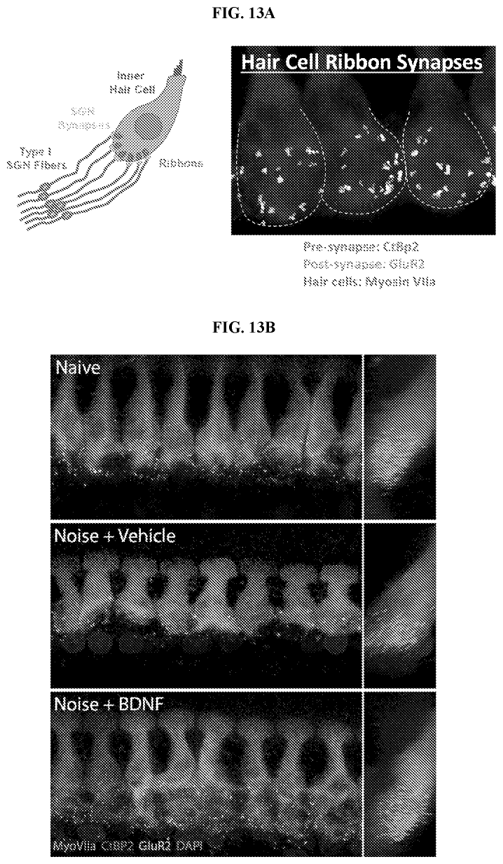

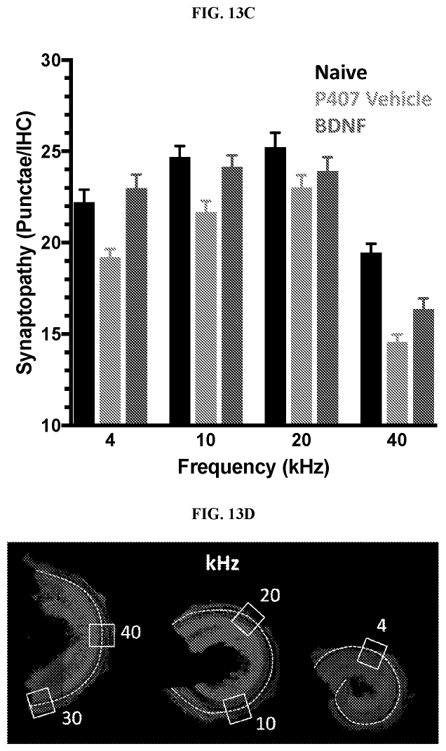

Intratympanic BDNF repairs cochlear synaptopathy in adult rats. Functional inner hair cell synapses are defined by colocalization of pre-synaptic and post-synaptic proteins associated with the inner hair cells and SGNs, respectively. FIG. 13A shows inner hair cell innervation (upper panel) and hair cell ribbon synapses (lower panel). FIG. 13B shows high-magnification views of inner hair cells from the 40 kHz region from adult rats after BDNF or vehicle treatment following noise damage, or in a naive undamaged cochlea. Digital X-compressed cross-sections are shown at right.

As shown in FIG. 13C, increased synaptic puncta (defined by co-expression of CtBp2 and GluR2) per inner hair cell were observed for each frequency region throughout the cochlea following BDNF treatment. As shown in FIG. 13D, the total length of the dissected cochlea was measured and then frequencies along the tonotopic axis were mapped for assessment.

### Example H—Study Design for Growth Factor Formulations

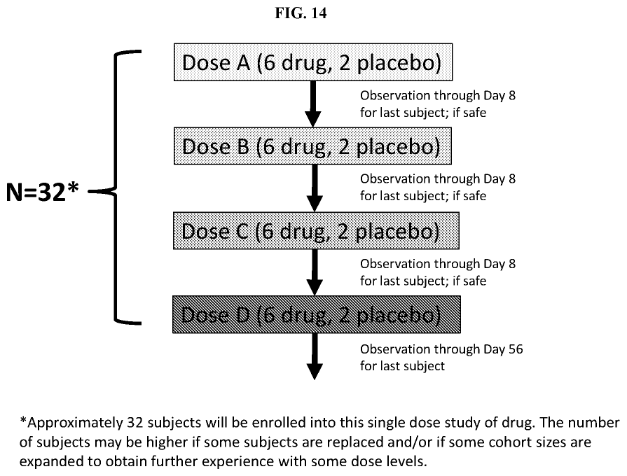

A randomized, double-blind, placebo-controlled study is performed in subjects with hearing impairment characterized by speech-in-noise deficits and mild-to-moderate age-related hearing loss. A single ascending is used for 4 dose level cohorts of 8 subjects each. Of the 8 subjects of each dose level cohort, 6 were assigned a therapeutic agent and 2 were assigned a placebo. The study is performed to assess the safety, pharmacokinetics, and exploratory efficacy of intratympanic therapeutic composition. Study outcomes include safety (e.g., otoscopy, tympanometry, vital signs, clinical safety labs, adverse events, suicidality), plasma pharmacokinetics, synaptopathy assessments (e.g., middle ear muscle reflex, ABR Wave 1/5 ratio, envelope following response), speech-in-noise tests, and hearing handicap questionnaire. FIG. 14 schematically depicts the scheme for this study. The therapeutic composition will be configured to otic administration and will include a growth factor and an auris acceptable vehicle such as a thermoreversible gel.

### Example I—Doses for Growth Factor Formulations

Good laboratory practice toxicology doses for otic formulations comprising a growth factor range from a low dose of about 0.05% (about 0.5 mg/ml), to a medium dose of about 0.15% (about 1.5 mg/ml), to a higher dose about 0.5% (about 5 mg/ml) of the growth factor. In some cases, the growth factor is BDNF. The remainder of the formulation includes auris acceptable vehicle such as a thermoreversible gel and optional other components, as described herein. In some cases, preparing such doses requires a 50 mg/ml growth factor (e.g., BDNF) concentrate. Such a concentrate may have limited stability over time.

Clinical doses range from about 0.01% to about 0.25% (e.g., about 0.1 mg/ml to about 2.5 mg/ml) of growth factor (e.g., BDNF). In some cases, preparing such clinical doses (e.g., for use in a Phase 1 or Phase 2 clinical trial) may require a 10× growth factor concentrate of 1 mg/ml to 25 mg/ml.

Solubility and stability of growth factor formulations are evaluated for concentrates (e.g., BDNF concentrates) between 1 mg/ml to 15 mg/ml to determine the highest usable concentrations. Solubility of growth factor (e.g., BDNF) is assessed in three buffer systems, phosphate buffer (PB), phosphate buffered saline (PBS), and Tris, to determine concentration limits of active vial concentrates.

The injection volume for an otic formulation comprising a growth factor is about 0.2 ml.

### Example J—Sustained Release of BDNF in Poloxamer Formulations (Feline PK)

BDNF in Poloxamer 407. Poloxamer 407 solution at 16% was prepared by slowly adding it to a cold buffer solution (50 mM tris buffer with saline, pH 7.4). Sterilization was achieved by filtration. Human recombinant BDNF was formulated as a concentrate with the same tris buffer solution to a concentration 10× the target concentration. The solutions were sterilized by filtration. Combining the above two solutions at 1:10 ratio yields a solution or a suspension containing 16% P407 and a concentration of 0.05% and 0.5% BDNF.

Pharmacokinetics. Adult male cats (Liberty Research) weighing 2-4 kg served as subjects (N=3 per group). Prior to any procedures, animals were anesthetized using a combination of xylazine (0.11 mg/kg) and ketamine (20 mg/kg) for up to an hour via the intramuscular route. If needed, an intraoperative booster was administered intramuscularly representing a one-tenth of the original dose.

Intratympanic injection. Each animal was positioned so that the head was tilted at an angle to favor injection towards the round window niche. Briefly, under visualization with an operating microscope, 150 μL of the formulation was injected using a 25G (Gauge) 11/2 needle through the tympanic membrane into the superior posterior quadrant. Formulations were delivered using a perfusion pump at the rate of 2 μL/sec. Contact with the round window membrane was maintained for 30 minutes by placing the animal in a recumbent position. During the procedure and until recovery, animals were placed on a temperature controlled (40° C.) heating pad until consciousness was regained at which time they were returned to the vivarium.

Perilymph sampling procedure. The skin extending from the ventral neck to the sternal notch was shaved and disinfected with povidone-iodine. An incision was then made medially and parallel with the digastric muscle in the ventral aspect of the neck, which is directly over the hollow bulla (the tympanic part of the temporal bone). The skin, subcutaneous tissue, platysma, and mylohyoid muscle were incised. The bulla was then exposed by blunt dissection between the digastric, styloglossus, and hyoglossus muscles. A 2-cm-diameter access site in the bulla was made using a rotary burr tool mounted on a dental drill. The mucosa internal to the bulla was freed to allow visualization of the round window in the posterolateral wall of the bulla. The location of round window was verified by identification of the promontory and the attachment of the stapes to the oval window. A 1 mm diameter hole was drilled with a diamond burr under a surgical microscope in the basal turn, and a microcapillary tube was used to collect perilymph (20 μl).

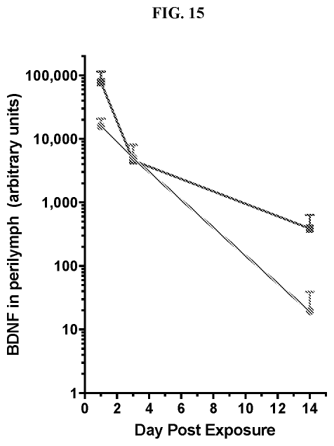

Analysis. Concentrations of BDNF in perilymph and samples were determined using commercially available ELISA kits. The limits of detection of human BDNF were 4 pg/mL. FIG. 15 shows perilymph concentrations of BDNF following a single intratympanic injection in cats of BDNF poloxamer 407 formulation. BDNF was administered at the indicated doses. Perilymph BDNF is in arbitrary units.

### Example K—ABR, Wave I Amplitude, and Synaptic Punctae of Various Concentrations of BDNF in P407

Animals: Male Sprague-Dawley rats (5-6 months of age) were used for efficacy studies and females (2-3 months) were used for pharmacokinetic studies.

Formulations: Human recombinant BDNF was suspended in the thermoreversible polymer Poloxamer 407 (16%) to reach a range of BDNF concentrations (0.005%, 0.05%, and 0.5% by weight).

Intratympanic (IT) injection: Formulations were administered under anesthesia as a single IT injection (20 μl) through the tympanic membrane into the middle ear, targeting the round window membrane (RWM).

Inner ear pharmacokinetics: Animals received a single unilateral intratympanic injection of the different treatment doses. BDNF levels in the perilymph were determined at the indicated times by ELISA.

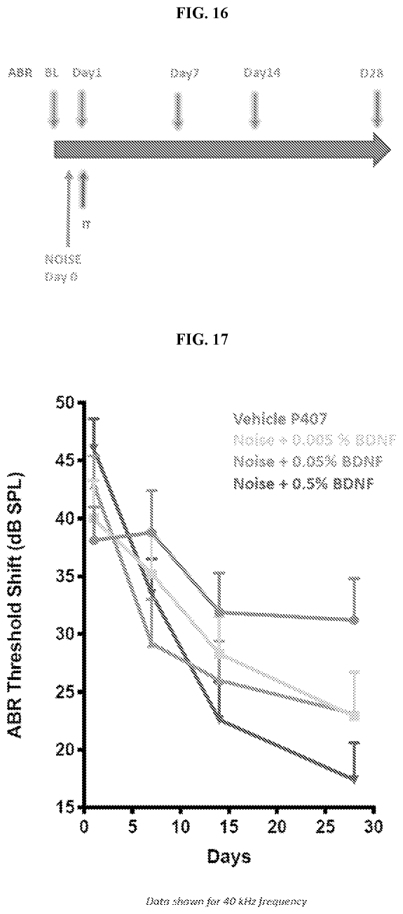

Noise-induced cochlear synaptopathy: Rats received a single acoustic trauma (105 dB SPL, 8-16 kHz, 1 h) under anesthesia. 24 hours later (Day 1) animals received a single bilateral IT injection of the treatment regimens. Auditory function (ABR and ABR wave 1 amplitude) was monitored at day 1 and then every 7 days for 28 days, as illustrated in FIG. 16. At termination (Day 28), cochleae were collected and assessed for evidence of cochlear synaptopathy (IHC synaptic puncta counts).

Immunohistochemistry: Dissected cochleae were immunostained with antibodies against presynaptic (CtBP2) and postsynaptic (GluR2) proteins and MyosinVIIa to label hair cells. Cochleae were then frequency mapped and high-magnification images were obtained with a Zeiss LSM-880 confocal microscope and imaged at an interval of 0.35 μm.

Intratympanic BDNF improves Auditory Brainstem Response (ABR) thresholds and wave I amplitudes in adult rats. The Auditory Brainstem Response (ABR) is an objective test that measures hearing sensitivity and can identify abnormalities in the peripheral auditory system and the auditory pathway up through the brainstem. Adult male rats were bilaterally exposed to noise followed by an intratympanic injection of BDNF in P407 24 hours after noise exposure. ABR recordings were obtained at days 1, 7, 14, and 28. FIG. 17 shows ABR threshold shift (dB SPL) at 40 Hz frequency for P407 vehicle alone and 0.005%, 0.05%, 0.5% BDNF in P407. At all three BDNF concentrations, BDNF-treated animals showed a more rapid recovery of initial threshold shifts and overall reduced levels of hearing loss.

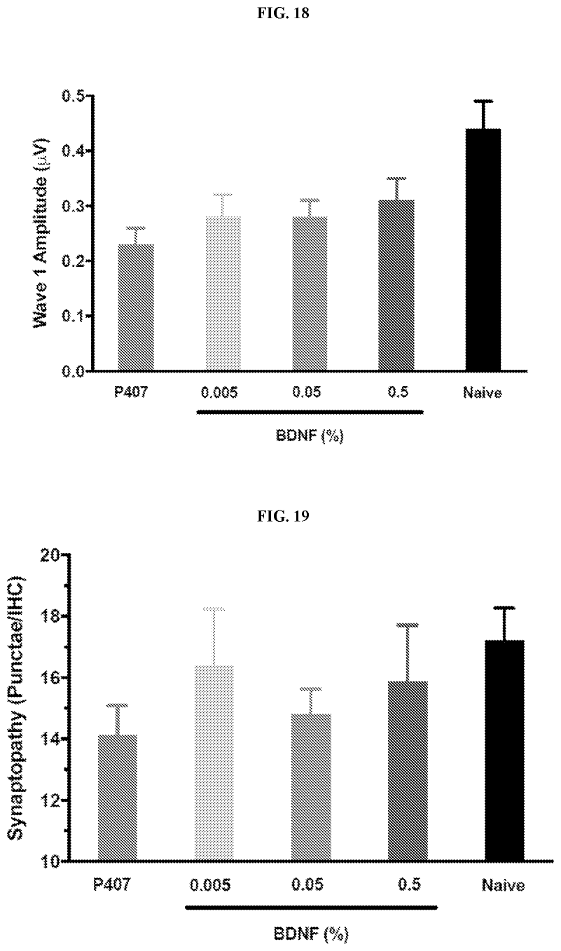

The ABR Wave I amplitude is an indicator of the strength of the distal cochlear nerve signal, i.e., the response of the spiral ganglion neurons/synapses. FIG. 18 shows wave I amplitudes at 40 Hz frequency for P407 vehicle alone and 0.005%, 0.05%, 0.5% BDNF in P407. At all three BDNF concentrations, significant improvements in wave I amplitudes were detected in BDNF-treated animals.

As shown in FIG. 19, increased synaptic puncta (defined by co-expression of CtBp2 and GluR2) per inner hair cell were observed for 40 Hz frequency throughout the cochlea following BDNF treatment at all three BDNF concentrations.

### Example L—ABR and Wave I Amplitude of Delayed Intratympanic Injection of BDNF in P407

Animals: Male Sprague-Dawley rats (5-6 months of age) were used for efficacy studies and females (2-3 months) were used for pharmacokinetic studies.

Formulations: Human recombinant BDNF was suspended in the thermoreversible polymer Poloxamer 407 (16%) to reach a BDNF concentration of 0.005%.

Intratympanic (IT) injection: Formulations were administered under anesthesia as a single IT injection (20 μl) through the tympanic membrane into the middle ear, targeting the round window membrane (RWM).

Inner ear pharmacokinetics: Animals received a single unilateral intratympanic injection of the different treatment doses. BDNF levels in the perilymph were determined at the indicated times by ELISA.

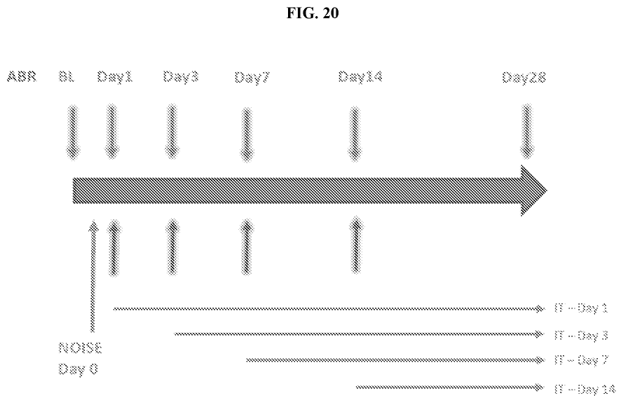

Noise-induced cochlear synaptopathy: Rats received a single acoustic trauma (105 dB SPL, 8-16 kHz, 1 h) under anesthesia. As illustrated in FIG. 20, a single bilateral intratympanic injection was made 1 day, 3 days, 7 days, or 14 day after onset of the acoustic trauma. Auditory function (ABR and ABR wave 1 amplitude) was monitored at day 28. At termination (Day 28), cochleae were collected and assessed for evidence of cochlear synaptopathy (IHC synaptic puncta counts).

Immunohistochemistry: Dissected cochleae were immunostained with antibodies against presynaptic (CtBP2) and postsynaptic (GluR2) proteins and MyosinVIIa to label hair cells. Cochleae were then frequency mapped and high-magnification images were obtained with a Zeiss LSM-880 confocal microscope and imaged at an interval of 0.35 μm.

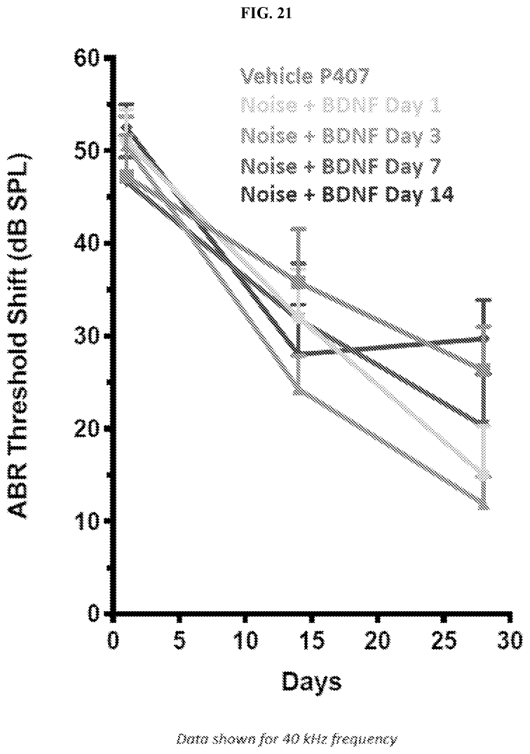

Intratympanic BDNF Injected 1, 3, or 7 Days After Acoustic Trauma improves Auditory Brainstem Response (ABR) thresholds and wave I amplitudes in adult rats. The Auditory Brainstem Response (ABR) is an objective test that measures hearing sensitivity and can identify abnormalities in the peripheral auditory system and the auditory pathway up through the brainstem. Adult male rats were bilaterally exposed to noise followed by an intratympanic injection of 0.005% BDNF in P407 1 day, 3 days, 7 days, or 14 days after noise exposure. ABR recordings were obtained at day 28. FIG. 21 shows ABR threshold shift (dB SPL) at 40 Hz frequency for P407 vehicle alone and 0.005% BDNF in P407 injected 1, 3, 7, or 14 days after noise exposure. Animals receiving BDNF injection 1, 3, or 7 days after noise exposure showed a more rapid recovery of initial threshold shifts and overall reduced levels of hearing loss. Animals receiving BDNF injection 14 days after noise exposure did not showed a more rapid recovery of initial threshold shifts and overall reduced levels of hearing loss.

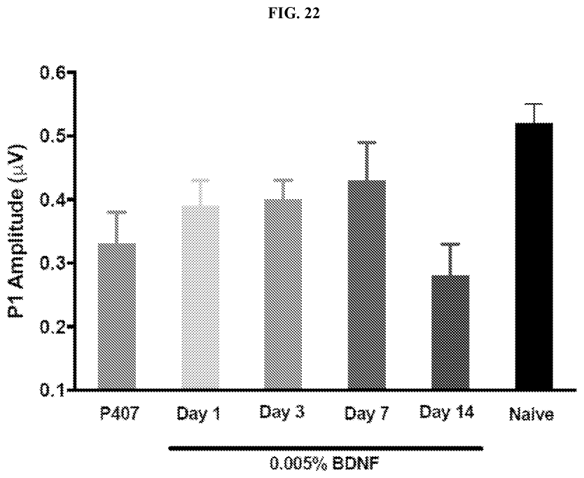

The ABR Wave I amplitude is an indicator of the strength of the distal cochlear nerve signal, i.e., the response of the spiral ganglion neurons/synapses. FIG. 22 shows wave I amplitudes at 40 Hz frequency for for P407 vehicle alone and 0.005% BDNF in P407 injected 1, 3, 7, or 14 days after noise exposure. Animals receiving BDNF injection 1, 3, or 7 days after noise exposure showed significant improvements in wave I amplitudes. Animals receiving BDNF injection 14 days after noise exposure did not show significant improvements in wave I amplitudes.

While preferred embodiments of the present disclosure have been shown and described herein, such embodiments are provided by way of example only. It is not intended that the invention be limited by the specific examples provided within the specification. Various alternatives to the embodiments described herein are optionally employed.

Furthermore, it shall be understood that all aspects of the invention are not limited to the specific depictions, configurations or relative proportions set forth herein which depend upon a variety of conditions and variables. It is intended that the following claims define the scope of the disclosure and that methods and structures within the scope of these claims and their equivalents be covered thereby.

What is claimed is:

1. A method of treating an otic disease or condition associated with a synapses-affecting event in a subject in need thereof, the method comprising administering an otic formulation comprising a therapeutically effective amount of a growth factor and an auris-acceptable vehicle within 14 days after onset of the synapses-damaging event, wherein the otic formulation is formulated to provide sustained release of the growth factor into the inner ear to promote formation of synapses or repaire damaged synapses.

2. The method of claim 1, wherein the growth factor is brain-derived neurotrophic factor (BDNF), ciliary neurotrophic factor (CNTF), glial cell-line derived neurotrophic factor (GDNF), neurotrophin-3, neurotrophin-4, or any combination thereof.

3. The method of claim 1 or claim 2, wherein the growth factor is brain-derived neurotrophic factor (BDNF).

4. The method of any one of claim 1, wherein the auris-acceptable gel comprises a copolymer of polyoxyethylene and polyoxypropylene.

5. The method of claim 4, wherein the copolymer of polyoxyethylene and polyoxypropylene is poloxamer 407.

6. The method of claim 1, wherein the otic formulation comprises from about 15 wt % to about 17 wt % poloxamer 407.

7. The method of claim 1, wherein the otic formulation has an osmolarity from about 100 mOsm/L to about 1000 mOsm/L.

8. The method of claim 1, wherein the otic formulation has a pH from about 7.0 to about 8.0.

9. The method of claim 1, wherein the otic formulation comprises about 0.0001% to about 1% by weight of the growth factor.

10. The method of claim 1, wherein the otic formulation comprises between about 0.005% to about 0.5% by weight of the growth factor.

11. The method of claim 1, wherein the otic formulation provides sustained release of the growth factor over a period of at least 3 days.

12. The method of claim 1, wherein the growth factor is dissolved in the otic formulation.

13. The method of claim 1, wherein the otic formulation is administered through intratympanic injection.

14. The method of claim 13, wherein the otic formulation is deposited on or close to the round window membrane of the subject.

15. The method of claim 1, wherein the otic disease or condition is hearing loss or hearing impairment.

16. The method of claim 1, wherein the synapses-affecting event is selected from the group consisting of: head trauma, traumatic brain injury (TBI), acoustic trauma, cochlear implant surgery, sudden sensorineural hearing loss, drug-induced ototoxicity, and excitotoxicity.

17. The method of claim 16, wherein the drug-induced ototoxicity is chemotherapy-induced ototoxicity.

18. The method of claim 16, wherein the drug-induced ototoxicity is cisplatin-induced ototoxicity.

19. The method of claim 16, wherein the drug-induced ototoxicity is antibiotic-induced ototoxicity.

20. The method of claim 1, wherein the otic formulation repairs ribbon synapses.

21. The method of claim 1, wherein the otic formulation is administered within 7 days after onset of the synapses-damaging event.

22. The method of claim 1, wherein the otic formulation is administered within 3 days after onset of the synapses-damaging event.

23. The method of claim 1, wherein the otic formulation is administered within 1 day after onset of the synapses-damaging event.
